SEQUENCE LISTING (128 sequences)

SEQ ID NO: 1 - DNA, 2214 bp, synthetic construct
  misc_feature 1..2214: note = AAVrh79
  source 1..2214: mol_type = other DNA
  CDS 1..2214: note = AAVrh79
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgatcccccgactcctccacgggcatcggcaaaaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaatgggacatcgggaggaagcaccaacgacaacacctacttcggctacagcaccccctgggggtattttgacttcaacagattccactgtcacttctcaccacgtgactggcagagactcatcaacaacaactggggattccggcccaagagactcagcttcaagctcttcaacatccaggttaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaacggaagtcaagccgtaggccgttcctcattctactgcctggaatattttccatctcaaatgctgcggactggaaacaactttgaatttagctacacctttgaggacgtgcccttccacagcagctacgcacacagccagagcctggaccggctgatgaaccctctcatcgaccagtacctgtattacctatccagaactcagtccacaggaggaactcaaggtacacagcaattgttattttctcaagccgggcctgcaaatatgtcggctcaggccaagaactggctacctggaccttgctaccggcagcagcgagtctccacgacactgtcgcaaaacaacaacagcaactttgcttggactggtgccacgaaatatcatctgaacggaagagactctttggtgaatcccggtgttgctatggcaacgcataaggacgacgaggaacgtttctttccatcgagcggagtcctgatgtttggaaaacagggtgctggaagagacaatgtggactatagcagcgttatgctaaccagcgaggaagaaatcaagaccactaaccctgtagccactgaacaatacggcgtggtggctgataacttgcagcaaaccaatacaggacctatcgtgggaaatgtcaacagccaaggagccttacctggcatggtctggcagaaccgagacgtgtacctgcagggtcccatttgggccaagattcctcacacggacggcaactttcacccgtctcctctgatgggcggctttggactgaaacacccgcctcctcaaatcctgatcaagaacactcccgttcctgcggatcctccaacgacgttcagccaggcgaaattggcttccttcatcacgcagtatagtaccggccaggtcagcgtggagatcgagtgggagctgcagaaggagaacagcaagcgctggaacccagaaattcagtatacttccaactactacaaatctacaaatgtggactttgctgtcaataccgagggtacatattcagagcctcgccccattggaactcgttacctcacccgtaatctg

SEQ ID NO: 2 - protein, 738 aa, synthetic construct
  REGION 1..738: note = Synthetic Construct
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 3 - DNA, 2208 bp, synthetic construct
  misc_feature 1..2208: note = AAV9
  source 1..2208: mol_type = other DNA
  CDS 1..2208: note = AAV9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 4 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 2211 bp, synthetic construct
  misc_feature 1..2211: note = AAVhu31
  source 1..2211: mol_type = other DNA
  CDS 1..2211: note = AAVhu31
atggctgccgatggttatcttccagattggctcgaggacactctctctgaaggaataagacagtggtggaagctcaaacctggccccccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaggccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtccggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttagtactgaaggtgtatatagtgaaccccgccccattggcaccagatacctgactcgtaatctgtaa

SEQ ID NO: 6 - protein, 736 aa, synthetic construct
  REGION 1..736: note = Synthetic Construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 7 - DNA, 2211 bp, synthetic construct
  misc_feature 1..2211: note = AAVhu32
  source 1..2211: mol_type = other DNA
  CDS 1..2211: note = AAVhu32
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 8 - protein, 736 aa, synthetic construct
  REGION 1..736: note = Synthetic Construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - DNA, 2178 bp, synthetic construct
  misc_feature 1..2178: note = AAV5.5.9
  source 1..2178: mol_type = other DNA
  CDS 1..2178: note = AAV5.5.9
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 10 - protein, 726 aa, synthetic construct
  REGION 1..726: note = Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - DNA, 2229 bp, synthetic construct
  misc_feature 1..2229: note = AAVPHP.B
  source 1..2229: mol_type = other DNA
  CDS 1..2229: note = AAVPHP.B
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 12 - protein, 743 aa, synthetic construct
  REGION 1..743: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 2208 bp, synthetic construct
  misc_feature 1..2208: note = AAVhu68
  source 1..2208: mol_type = other DNA
  CDS 1..2208: note = AAVhu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctg

SEQ ID NO: 14 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2211 bp, synthetic construct
  misc_feature 1..2211: note = AAV8T
  source 1..2211: mol_type = other DNA
  CDS 1..2211: note = AAV8T
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctcctctggtactcatggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggtgggagtaggcctacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtgggtgataacttgcagttgtataacacggctcctggttcggtgtttgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 16 - protein, 737 aa, synthetic construct
  REGION 1..737: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSGTHGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGSRPTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVGDNLQLYNTAPGSVFVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 17 - DNA, 2214 bp, synthetic construct
  misc_feature 1..2214: note = AAV8.AR2.08
  source 1..2214: mol_type = other DNA
  CDS 1..2214: note = AAV8.AR2.08
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtgtgtgataacttgcagagtcggaacacggctcctcgtgaggagattgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 18 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVCDNLQSRNTAPREEIVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - DNA, 2214 bp, synthetic construct
  misc_feature 1..2214: note = AAV8
  source 1..2214: mol_type = other DNA
  CDS 1..2214: note = AAV8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 20 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - DNA, 2214 bp, synthetic construct
  misc_feature 1..2214: note = AAVhu37
  source 1..2214: mol_type = other DNA
  CDS 1..2214: note = AAVhu37
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgttcggaaaacagggtgctggaagagacaatgtggactacagcagcgttatgctaaccagcgaagaagaaattaaaaccactaaccccgtagccacagaacaatacggtgtggtggctgacaacttgcagcaaaccaatacagggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 22 - protein, 738 aa, synthetic construct
  REGION 1..738: note = Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 23 - DNA, 2214 bp, synthetic construct
  misc_feature 1..2214: note = AAVrh10
  source 1..2214: mol_type = other DNA
  CDS 1..2214: note = AAVrh10
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctg

SEQ ID NO: 24 - protein, 738 aa, synthetic construct
  REGION 1..738: note = Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 25 - protein, 33 aa, synthetic construct
  REGION 1..33: note = AAV9
QRVSTTVTQNNNSEFAWPGASSWALNGRNSLMN

SEQ ID NO: 26 - protein, 33 aa, synthetic construct
  REGION 1..33: note = AAV8
QRVSTTTGQNNNSNFAWTAGTKYHLNGRNSLAN

SEQ ID NO: 27 - protein, 33 aa, synthetic construct
  REGION 1..33: note = AAVrh10
QRVSTTLSQNNNSNFAWTGATKYHLNGRDSLVN

SEQ ID NO: 28 - protein, 33 aa, synthetic construct
  REGION 1..33: note = AAV3B
QRLSKTANDNNNSNFPWTAASKYHLNGRDSLVN

SEQ ID NO: 29 - protein, 33 aa, synthetic construct
  REGION 1..33: note = AAV2
QRVSKTSADNNNSEYSWTGATKYHLNGRDSLVN

SEQ ID NO: 30 - protein, 33 aa, synthetic construct
  REGION 1..33: note = AAV9
YGQVATNHQSAQAQAQTGWVQNQGILPGMVWQD

SEQ ID NO: 31 - protein, 33 aa, synthetic construct
  REGION 1..33: note = AAV8
YGIVADNLQQQNTAPQIGTVNSQGALPGMVWQN

SEQ ID NO: 32 - protein, 33 aa, synthetic construct
  REGION 1..33: note = AAVrh10
YGVVADNLQQQNAAPIVGAVNSQGALPGMVWQN

SEQ ID NO: 33 - protein, 33 aa, synthetic construct
  REGION 1..33: note = AAV3B
YGTVANNLQSSNTAPTTRTVNDQGALPGMVWQD

SEQ ID NO: 34 - protein, 33 aa, synthetic construct
  REGION 1..33: note = AAV2
YGSVSTNLQRGNRQAATADVNTQGVLPGMVWQD

SEQ ID NO: 35 - protein, 736 aa, synthetic construct
  REGION 1..736: note = AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 36 - protein, 736 aa, synthetic construct
  REGION 1..736: note = AAV3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 37 - protein, 724 aa, synthetic construct
  REGION 1..724: note = AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 38 - protein, 737 aa, synthetic construct
  REGION 1..737: note = AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 39 - protein, 733 aa, synthetic construct
  REGION 1..733: note = AAVrh32.33
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 40 - DNA, 2214 bp, synthetic construct
  misc_feature 1..2214: note = AVV mutant 8G264AG515A
  source 1..2214: mol_type = other DNA
  CDS 1..2214: note = AVV mutant 8G264AG515A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 41 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 42 - DNA, 2214 bp, synthetic construct
  misc_feature 1..2214: note = AVV mutant 8G264AG541A
  source 1..2214: mol_type = other DNA
  CDS 1..2214: note = AVV mutant 8G264AG541A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 43 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 44 - DNA, 2214 bp, synthetic construct
  misc_feature 1..2214: note = AVV mutant 8G515AG541A
  source 1..2214: mol_type = other DNA
  CDS 1..2214: note = AVV mutant 8G515AG541A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 45 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 46 - DNA, 2214 bp, synthetic construct
  misc_feature 1..2214: note = AVV mutant 8G264AG515AG541A
  source 1..2214: mol_type = other DNA
  CDS 1..2214: note = AVV mutant 8G264AG515AG541A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 47 - protein, 738 aa, synthetic construct
  REGION 1..738: note = Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 48 - DNA, 2211 bp, synthetic construct
  misc_feature 1..2211: note = AVV mutant 9G330AG453A
  source 1..2211: mol_type = other DNA
  CDS 1..2211: note = AVV mutant 9G330AG453A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 49 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - DNA, 2211 bp, synthetic construct
  misc_feature 1..2211: note = AVV mutant 9G330AG513A
  source 1..2211: mol_type = other DNA
  CDS 1..2211: note = AVV mutant 9G330AG513A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 51 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - DNA, 2211 bp, synthetic construct
  misc_feature 1..2211: note = AVV mutant 9G453AG513A
  source 1..2211: mol_type = other DNA
  CDS 1..2211: note = AVV mutant 9G453AG513A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 53 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - DNA, 2211 bp, synthetic construct
  misc_feature 1..2211: note = AVV mutant 9G330AG453AG513A
  source 1..2211: mol_type = other DNA
  CDS 1..2211: note = AVV mutant 9G330AG453AG513A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 55 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - DNA, 38 bp, synthetic construct
  misc_feature 1..38: note = primer sequence
  source 1..38: mol_type = other DNA
cgacaaccgggcaaaaccagaatagcaactttgcctgg

SEQ ID NO: 57 - DNA, 38 bp, synthetic construct
  misc_feature 1..38: note = primer sequence
  source 1..38: mol_type = other DNA
ccaggcaaagttgctattctggttttgcccggttgtcg

SEQ ID NO: 58 - DNA, 36 bp, synthetic construct
  misc_feature 1..36: note = primer sequence
  source 1..36: mol_type = other DNA
gacaaccgggcaaaacgacaatagcaactttgcctg

SEQ ID NO: 59 - DNA, 36 bp, synthetic construct
  misc_feature 1..36: note = primer sequence
  source 1..36: mol_type = other DNA
caggcaaagttgctattgtcgttttgcccggttgtc

SEQ ID NO: 60 - DNA, 29 bp, synthetic construct
  misc_feature 1..29: note = primer sequence
  source 1..29: mol_type = other DNA
ggaggcacggcacagacgcagactctggg

SEQ ID NO: 61 - DNA, 29 bp, synthetic construct
  misc_feature 1..29: note = primer sequence
  source 1..29: mol_type = other DNA
cccagagtctgcgtctgtgccgtgcctcc

SEQ ID NO: 62 - DNA, 30 bp, synthetic construct
  misc_feature 1..30: note = primer sequence
  source 1..30: mol_type = other DNA
caggaggcacggcagatacgcagactctgg

SEQ ID NO: 63 - DNA, 30 bp, synthetic construct
  misc_feature 1..30: note = primer sequence
  source 1..30: mol_type = other DNA
ccagagtctgcgtatctgccgtgcctcctg

SEQ ID NO: 64 - DNA, 29 bp, synthetic construct
  misc_feature 1..29: note = primer sequence
  source 1..29: mol_type = other DNA
ctcctcccgatgtcgcgttggagatttgc

SEQ ID NO: 65 - DNA, 29 bp, synthetic construct
  misc_feature 1..29: note = primer sequence
  source 1..29: mol_type = other DNA
gcaaatctccaacgcgacatcgggaggag

SEQ ID NO: 66 - DNA, 31 bp, synthetic construct
  misc_feature 1..31: note = primer sequence
  source 1..31: mol_type = other DNA
cccacggcctgactagcgttgttgagtgtta

SEQ ID NO: 67 - DNA, 31 bp, synthetic construct
  misc_feature 1..31: note = primer sequence
  source 1..31: mol_type = other DNA
taacactcaacaacgctagtcaggccgtggg

SEQ ID NO: 68 - DNA, 43 bp, synthetic construct
  misc_feature 1..43: note = primer sequence
  source 1..43: mol_type = other DNA
ggattagccaatgaatttcttgcattcagatggtatttggtcc

SEQ ID NO: 69 - DNA, 43 bp, synthetic construct
  misc_feature 1..43: note = primer sequence
  source 1..43: mol_type = other DNA
ggaccaaataccatctgaatgcaagaaattcattggctaatcc

SEQ ID NO: 70 - DNA, 39 bp, synthetic construct
  misc_feature 1..39: note = primer sequence
  source 1..39: mol_type = other DNA
tttgccaaaaatcaggatcgcgttactgggaaaaaaacg

SEQ ID NO: 71 - DNA, 39 bp, synthetic construct
  misc_feature 1..39: note = primer sequence
  source 1..39: mol_type = other DNA
cgtttttttcccagtaacgcgatcctgatttttggcaaa

SEQ ID NO: 72 - DNA, 27 bp, synthetic construct
  misc_feature 1..27: note = primer sequence
  source 1..27: mol_type = other DNA
ggacccttcaacgcactcgacaagggg

SEQ ID NO: 73 - DNA, 27 bp, synthetic construct
  misc_feature 1..27: note = primer sequence
  source 1..27: mol_type = other DNA
ccccttgtcgagtgcgttgaagggtcc

SEQ ID NO: 74 - DNA, 35 bp, synthetic construct
  misc_feature 1..35: note = primer sequence
  source 1..35: mol_type = other DNA
tggctcctcccgatgtgctgttggagatttgcttg

SEQ ID NO: 75 - DNA, 35 bp, synthetic construct
  misc_feature 1..35: note = primer sequence
  source 1..35: mol_type = other DNA
caagcaaatctccaacagcacatcgggaggagcca

SEQ ID NO: 76 - DNA, 33 bp, synthetic construct
  misc_feature 1..33: note = primer sequence
  source 1..33: mol_type = other DNA
cccacggcctgactactgttgttgagtgttagg

SEQ ID NO: 77 - DNA, 33 bp, synthetic construct
  misc_feature 1..33: note = primer sequence
  source 1..33: mol_type = other DNA
cctaacactcaacaacagtagtcaggccgtggg

SEQ ID NO: 78 - DNA, 46 bp, synthetic construct
  misc_feature 1..46: note = primer sequence
  source 1..46: mol_type = other DNA
ttagccaatgaatttctgctattcagatggtatttggtcccagcag

SEQ ID NO: 79 - DNA, 46 bp, synthetic construct
  misc_feature 1..46: note = primer sequence
  source 1..46: mol_type = other DNA
ctgctgggaccaaataccatctgaatagcagaaattcattggctaa

SEQ ID NO: 80 - DNA, 45 bp, synthetic construct
  misc_feature 1..45: note = primer sequence
  source 1..45: mol_type = other DNA
ttgtttgccaaaaatcaggatgctgttactgggaaaaaaacgctc

SEQ ID NO: 81 - DNA, 45 bp, synthetic construct
  misc_feature 1..45: note = primer sequence
  source 1..45: mol_type = other DNA
gagcgtttttttcccagtaacagcatcctgatttttggcaaacaa

SEQ ID NO: 82 - DNA, 33 bp, synthetic construct
  misc_feature 1..33: note = primer sequence
  source 1..33: mol_type = other DNA
ctcccccttgtcgaggctgttgaagggtccgag

SEQ ID NO: 83 - DNA, 33 bp, synthetic construct
  misc_feature 1..33: note = primer sequence
  source 1..33: mol_type = other DNA
ctcggacccttcaacagcctcgacaagggggag

SEQ ID NO: 84 - DNA, 32 bp, synthetic construct
  misc_feature 1..32: note = primer sequence
  source 1..32: mol_type = other DNA
cagcgactcatcaacgacaactggggattccg

SEQ ID NO: 85 - DNA, 28 bp, synthetic construct
  misc_feature 1..28: note = primer sequence
  source 1..28: mol_type = other DNA
ggaggcacggcagatacgcagactctgg

SEQ ID NO: 86 - DNA, 36 bp, synthetic construct; identical to SEQ ID NO: 58
  misc_feature 1..36: note = primer sequence
  source 1..36: mol_type = other DNA

SEQ ID NO: 87 - DNA, 40 bp, synthetic construct
  misc_feature 1..40: note = primer sequence
  source 1..40: mol_type = other DNA
ccatctgaatggaagagattcattggctaatcctggcatc

SEQ ID NO: 88 - DNA, 30 bp, synthetic construct
  misc_feature 1..30: note = primer sequence
  source 1..30: mol_type = other DNA
cgaagcccaaagccgaccagcaaaagcagg

SEQ ID NO: 89 - DNA, 28 bp, synthetic construct
  misc_feature 1..28: note = primer sequence
  source 1..28: mol_type = other DNA
gtacctgcggtatgaccacgccgacgcc

SEQ ID NO: 90 - DNA, 37 bp, synthetic construct
  misc_feature 1..37: note = primer sequence
  source 1..37: mol_type = other DNA
gatgctgagaaccggcgacaacttccagtttacttac

SEQ ID NO: 91 - DNA, 39 bp, synthetic construct
  misc_feature 1..39: note = primer sequence
  source 1..39: mol_type = other DNA
cagactctgggcttcagcgatggtgggcctaatacaatg

SEQ ID NO: 92 - DNA, 30 bp, synthetic construct
  misc_feature 1..30: note = primer sequence
  source 1..30: mol_type = other DNA
ccaatcaggcaaaggactggctgccaggac

SEQ ID NO: 93 - DNA, 26 bp, synthetic construct
  misc_feature 1..26: note = primer sequence
  source 1..26: mol_type = other DNA
cacggacggcgacttccacccgtctc

SEQ ID NO: 94 - DNA, 31 bp, synthetic construct
  misc_feature 1..31: note = primer sequence
  source 1..31: mol_type = other DNA
gatcctgatcaaggacacgcctgtacctgcg

SEQ ID NO: 95 - DNA, 33 bp, synthetic construct
  misc_feature 1..33: note = primer sequence
  source 1..33: mol_type = other DNA
gtacctcggacccttccagggactcgacaaggg

SEQ ID NO: 96 - DNA, 36 bp, synthetic construct
  misc_feature 1..36: note = primer sequence
  source 1..36: mol_type = other DNA
ctacaagcaaatctcccaggggacatcgggaggagc

SEQ ID NO: 97 - DNA, 36 bp, synthetic construct
  misc_feature 1..36: note = primer sequence
  source 1..36: mol_type = other DNA
gctacctaacactcaaccagggtagtcaggccgtgg

SEQ ID NO: 98 - DNA, 41 bp, synthetic construct
  misc_feature 1..41: note = primer sequence
  source 1..41: mol_type = other DNA
gctgggaccaaataccatctgcagggaagaaattcattggc

SEQ ID NO: 99 - DNA, 40 bp, synthetic construct
  misc_feature 1..40: note = primer sequence
  source 1..40: mol_type = other DNA
ggagcgtttttttcccagtcaggggatcctgatttttggc

SEQ ID NO: 100 - DNA, 32 bp, synthetic construct
  misc_feature 1..32: note = primer sequence
  source 1..32: mol_type = other DNA
cggaatccccagttgtcgttgatgagtcgctg

SEQ ID NO: 101 - DNA, 28 bp, synthetic construct
  misc_feature 1..28: note = primer sequence
  source 1..28: mol_type = other DNA
ccagagtctgcgtatctgccgtgcctcc

SEQ ID NO: 102 - DNA, 36 bp, synthetic construct; identical to SEQ ID NO: 59
  misc_feature 1..36: note = primer sequence
  source 1..36: mol_type = other DNA

SEQ ID NO: 103 - DNA, 40 bp, synthetic construct
  misc_feature 1..40: note = primer sequence
  source 1..40: mol_type = other DNA
gatgccaggattagccaatgaatctcttccattcagatgg

SEQ ID NO: 104 - DNA, 30 bp, synthetic construct
  misc_feature 1..30: note = primer sequence
  source 1..30: mol_type = other DNA
cctgcttttgctggtcggctttgggcttcg

SEQ ID NO: 105 - DNA, 28 bp, synthetic construct
  misc_feature 1..28: note = primer sequence
  source 1..28: mol_type = other DNA
ggcgtcggcgtggtcataccgcaggtac

SEQ ID NO: 106 - DNA, 37 bp, synthetic construct
  misc_feature 1..37: note = primer sequence
  source 1..37: mol_type = other DNA
gtaagtaaactggaagttgtcgccggttctcagcatc

SEQ ID NO: 107 - DNA, 39 bp, synthetic construct
  misc_feature 1..39: note = primer sequence
  source 1..39: mol_type = other DNA
cattgtattaggcccaccatcgctgaagcccagagtctg

SEQ ID NO: 108 - DNA, 30 bp, synthetic construct
  misc_feature 1..30: note = primer sequence
  source 1..30: mol_type = other DNA
gtcctggcagccagtcctttgcctgattgg

SEQ ID NO: 109 - DNA, 26 bp, synthetic construct
  misc_feature 1..26: note = primer sequence
  source 1..26: mol_type = other DNA
gagacgggtggaagtcgccgtccgtg

SEQ ID NO: 110 - DNA, 31 bp, synthetic construct
  misc_feature 1..31: note = primer sequence
  source 1..31: mol_type = other DNA
cgcaggtacaggcgtgtccttgatcaggatc

SEQ ID NO: 111 - DNA, 35 bp, synthetic construct
  misc_feature 1..35: note = primer sequence
  source 1..35: mol_type = other DNA
gcagcgactcatcaacgacaactggggattccggc

SEQ ID NO: 112 - DNA, 35 bp, synthetic construct
  misc_feature 1..35: note = primer sequence
  source 1..35: mol_type = other DNA
gccggaatccccagttgtcgttgatgagtcgctgc

SEQ ID NO: 113 - DNA, 34 bp, synthetic construct
  misc_feature 1..34: note = primer sequence
  source 1..34: mol_type = other DNA
cagcgactcatcaacgacaactggggattccggc

SEQ ID NO: 114 - DNA, 34 bp, synthetic construct
  misc_feature 1..34: note = primer sequence
  source 1..34: mol_type = other DNA
gccggaatccccagttgtcgttgatgagtcgctg

SEQ ID NO: 115 - DNA, 30 bp, synthetic construct
  misc_feature 1..30: note = primer sequence
  source 1..30: mol_type = other DNA
gcgactcatcaacgacaactggggattccg

SEQ ID NO: 116 - DNA, 30 bp, synthetic construct
  misc_feature 1..30: note = primer sequence
  source 1..30: mol_type = other DNA
cggaatccccagttgtcgttgatgagtcgc

SEQ ID NO: 117 - DNA, 31 bp, synthetic construct
  misc_feature 1..31: note = primer sequence
  source 1..31: mol_type = other DNA
ctctgggcttcagcgaaggtgggcctaatac

SEQ ID NO: 118 - DNA, 31 bp, synthetic construct
  misc_feature 1..31: note = primer sequence
  source 1..31: mol_type = other DNA
gtattaggcccaccttcgctgaagcccagag

SEQ ID NO: 119 - DNA, 29 bp, synthetic construct
  misc_feature 1..29: note = primer sequence
  source 1..29: mol_type = other DNA
cctcggacccttcgacggactcgacaagg

SEQ ID NO: 120 - DNA, 34 bp, synthetic construct
  misc_feature 1..34: note = primer sequence
  source 1..34: mol_type = other DNA
tacaagcaaatctccgacgggacatcgggaggag

SEQ ID NO: 121 - DNA, 34 bp, synthetic construct
  misc_feature 1..34: note = primer sequence
  source 1..34: mol_type = other DNA
ctacctaacactcaacgacggtagtcaggccgtg

SEQ ID NO: 122 - DNA, 45 bp, synthetic construct
  misc_feature 1..45: note = primer sequence
  source 1..45: mol_type = other DNA
ctgggaccaaataccatctggatggaagaaattcattggctaatc

SEQ ID NO: 123 - DNA, 39 bp, synthetic construct
  misc_feature 1..39: note = primer sequence
  source 1..39: mol_type = other DNA
gagcgtttttttcccagtgacgggatcctgatttttggc

SEQ ID NO: 124 - DNA, 29 bp, synthetic construct
  misc_feature 1..29: note = primer sequence
  source 1..29: mol_type = other DNA
ccttgtcgagtccgtcgaagggtccgagg

SEQ ID NO: 125 - DNA, 34 bp, synthetic construct
  misc_feature 1..34: note = primer sequence
  source 1..34: mol_type = other DNA
ctcctcccgatgtcccgtcggagatttgcttgta

SEQ ID NO: 126 - DNA, 34 bp, synthetic construct
  misc_feature 1..34: note = primer sequence
  source 1..34: mol_type = other DNA
cacggcctgactaccgtcgttgagtgttaggtag

SEQ ID NO: 127 - DNA, 45 bp, synthetic construct
  misc_feature 1..45: note = primer sequence
  source 1..45: mol_type = other DNA
gattagccaatgaatttcttccatccagatggtatttggtcccag

SEQ ID NO: 128 - DNA, 39 bp, synthetic construct
  misc_feature 1..39: note = primer sequence
  source 1..39: mol_type = other DNA
gccaaaaatcaggatcccgtcactgggaaaaaaacgctc

(19) United States
(12) Patent Application Publication
(10) Pub. No.: US 2026/0049334 A1
(43) Pub. Date: Feb. 19, 2026

(54) NOVEL ADENO-ASSOCIATED VIRUS (AAV) VECTORS, AAV VECTORS HAVING REDUCED CAPSID DEAMIDATION AND USES THEREFOR

(71) Applicant: The Trustees of the University of Pennsylvania, Philadelphia, PA (US)

(72) Inventors: James M. Wilson, Philadelphia, PA (US); April Tepe, Laurel, MD (US); Kevin Turner, Newtown Square, PA (US); Joshua Joyner Sims, Villanova, PA (US); Qiang Wang, Philadelphia, PA (US)

(73) Assignee: The Trustees of the University of Pennsylvania, Philadelphia, PA (US)

(21) Appl. No.: 19/297,827

(22) Filed: Aug. 12, 2025

Related U.S. Application Data
(63) Continuation of application No. 16/975,545, now Pat. No. 12,416,016.
(60) Provisional application No. 62/635,964, filed on Feb. 27, 2018.
(60) Provisional application No. 62/635,968, filed on Feb. 27, 2018.
(60) Provisional application No. 62/663,788, filed on Apr. 27, 2018.
(60) Provisional application No. 62/663,797, filed on Apr. 27, 2018.
(60) Provisional application No. 62/667,587, filed on May 6, 2018.
(60) Provisional application No. 62/667,888, filed on May 7, 2018.
(60) Provisional application No. 62/667,881, filed on May 7, 2018.
(60) Provisional application No. 62/677,471, filed on May 29, 2018.
(60) Provisional application No. 62/677,474, filed on May 29, 2018.
(60) Provisional application No. 62/667,585, filed on May 29, 2018.
(60) Provisional application No. 62/703,670, filed on Jul. 26, 2018.
(60) Provisional application No. 62/703,673, filed on Jul. 26, 2018.
(60) Provisional application No. 62/722,382, filed on Aug. 24, 2018.
(60) Provisional application No. 62/722,388, filed on Aug. 24, 2018.

(51) Int. Cl. C12N 15/86; C07K 14/005

(52) U.S. Cl. CPC C12N 15/86; C07K 14/005; C12N 2750/14121; C12N 2750/14122; C12N 2750/14143

(57) ABSTRACT

A recombinant adeno-associated virus (rAAV) vector comprising an AAV capsid having a heterogeneous population of vp1 proteins, a heterogeneous population of vp2 protein and a heterogeneous population of vp3 proteins. The capsid contains modified amino acids as compared to the encoded VP1 amino acid sequence, the capsid containing highly deamidated asparagine residues at asparagine-glycine pair, and further comprising multiple other, less deamidated asparagine and optionally glutamine residues.

### STATEMENT REGARDING FEDERALLY SPONSORED RESEARCH OR DEVELOPMENT

This invention was made with government support under P01HL059407 awarded by the National Institutes of Health. The government has certain rights in the invention.

### REFERENCE TO A SEQUENCE LISTING SUBMITTED VIA EFS-WEB

The content of the text filed of the sequence listing named “UPN-18-8592.US.C1_ST26_Sequence Listing.xml” which was created on Aug. 8, 2025 (file size 232,592 bytes) and electronically submitted via EFS-Web herewith the application is incorporated herein by reference in its entirety.

### BACKGROUND OF THE INVENTION

The adeno-associated virus (AAV) capsid is icosahedral in structure and is comprised of 60 of viral protein (VP) monomers (VP1, VP2, and VP3) in a 1:1:10 ratio (Xie Q, et al. Proc Natl Acad Sci USA. 2002; 99 (16): 10405-10). The entirety of the VP3 protein sequence (˜535aa) is contained within the C-terminus of both VP1 and VP2, and the shared VP3 sequences are primarily responsible for the overall capsid structure. Due to the structural flexibility of the VP1/VP2 unique regions and the low representation of VP1 and VP2 monomers relative to VP3 monomers in the assembled capsid, VP3 is the only capsid protein to be resolved via x-ray crystallography (Nam H J, et al. J Virol. 2007; 81 (22): 12260-71). VP3 contains nine hypervariable regions (HVRs) that are the primary source of sequence variation between AAV serotypes (Govindasamy L, et al. J Virol. 2013; 87 (20): 11187-99). Given their flexibility and location on the capsid surface, HVRs are largely responsible for interactions with target cells as well as with the immune system (Huang L Y, et al. J Virol. 2016; 90 (11): 5219-30; Raupp C, et al. J Virol. 2012; 86 (17): 9396-408). While the structures of a number of serotypes are published (Protein Data Bank (PDB) IDs ILP3, 4RSO, 4V86, 3UX1, 3KIC, 2QA0, 2G8G from the Research Collaboratory for Structural Bioinformatics (RCSB) database) for the structure entries for AAV2, AAVrh.8, AAV6, AAV9, AAV3B, AAV8, and AAV4, respectively), there is very little information in the literature regarding modifications on the surface of these capsids. Research suggests that intracellular phosphorylation of the capsid occurs at specific tyrosine residues (Zhong L, et al. Virology. 2008; 381 (2): 194-202). Despite putative glycosylation sites in the primary VP3 sequence, no glycosylation events have been identified in AAV2 (Murray S, et al. J Virol. 2006; 80 (12): 6171-6; Jin X, et al. Hum Gene Ther Methods. 2017; 28 (5): 255-267); other AAV serotypes have not yet been evaluated for capsid glycosylation.

AAV gene therapy vectors have undergone less of the molecular-level scrutiny that typically accompanies the development and manufacturing of recombinant protein therapeutics. AAV capsid post-translational modifications (PTM) have largely been unexplored, so accordingly, little is known about their potential to impact function, or about strategies to control PTM levels in manufactured AAV therapies.

Variations in post-translational modifications of non-gene therapy protein therapeutics have complicated their development as drugs. Jenkins, N, Murphy, L, and Tyther, R (2008). Post-translational modifications of recombinant proteins: significance for biopharmaceuticals. Mol Biotechnol 39:113-118; Houde, D, Peng, Y, Berkowitz, S A, and Engen, JR (2010). Post-translational modifications differentially affect IgG1 conformation and receptor binding. Mol Cell Proteomics 9:1716-1728. For example, deamidation of selected amino acids modulates the stability of and the immune response to the recombinant protective antigen-based anthrax vaccine. (Powell B S, et al. Proteins. 2007; 68 (2): 458-79; Verma A, et al. Clin Vaccine Immunol. 2016; 23 (5): 396-402). In some instances, this process is catalyzed by viral or bacterial deamidases to modulate host cell signaling pathways or innate immune responses (Zhao J, et al. J Virol. 2016; 90 (9): 4262-8; Zhao J, et al. Cell Host Microbe. 2016; 20 (6): 770-84). More commonly, endogenous deamidation is an enzyme-independent spontaneous process. Although the purpose of spontaneous deamidation has not been fully elucidated, previous studies have suggested that this event serves as a molecular clock to indicate the relative age of a protein and regulate its turnover (Robinson N E and Robinson A B. Proc Natl Acad Sci USA. 2001; 98 (3): 944-9).

Deamidation occurs when the amide group of asparagine or less frequently glutamine undergoes nucleophilic attack from an adjacent nitrogen atom and the amide group is lost. This process leads to a succinimidyl intermediate (Yang H and Zubarev R A. Electrophoresis. 2010; 31 (11): 1764-72) that, via hydrolysis, resolves into a mixture of aspartic acid and isoaspartic acid (or glutamic acid and isoglutamic acid) (Catak S, et al. J Phys Chem A. 2009; 113 (6): 1111-20). Studies of short, synthetic peptides estimate that this hydrolysis results in a 3:1 mixture of isoaspartic acid to aspartic acid (Geiger T. and Clarke S. J Biol Chem. 1987; 262 (2): 785-94.

There continues to be a need for compositions comprising AAV-based constructs for delivery of heterologous molecules which have stable receptor binding and/or stable capsids, avoid neutralizing antibodies and/or retain purity on storage.

### SUMMARY OF THE INVENTION

In one embodiment, a composition is provided which includes a mixed population of recombinant adeno-associated virus (rAAV), each of said rAAV comprising: (a) an AAV capsid comprising about 60 capsid vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine-glycine pairs in the AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the deamidated asparagines are deamidated to aspartic acid, isoaspartic acid, an interconverting aspartic acid/isoaspartic acid pair, or combinations thereof. In certain embodiments, the capsid further comprises deamidated glutamine(s) which are deamidated to (α)-glutamic acid, γ-glutamic acid, an interconverting (α)-glutamic acid/γ-glutamic acid pair, or combinations thereof.

In a further aspect, a recombinant adeno-associated virus (rAAV) is provided which comprises: (A) an AAVrh79 capsid comprising one or more of: (1) AAVrh79 capsid proteins comprising: a heterogeneous population of AAVrh79 vp1 proteins selected from: vp1 proteins produced by expression from a nucleic acid sequence which encodes the predicted amino acid sequence of 1 to 738 of SEQ ID NO:2, vp1 proteins produced from SEQ ID NO: 1, or vp1 proteins produced from a nucleic acid sequence at least 70% identical to SEQ ID NO: 1 which encodes the predicted amino acid sequence of 1 to 738 of SEQ ID NO:2, a heterogeneous population of AAVrh79 vp2 proteins selected from: vp2 proteins produced by expression from a nucleic acid sequence which encodes the predicted amino acid sequence of at least about amino acids 138 to 738 of SEQ ID NO:2, vp2 proteins produced from a sequence comprising at least nucleotides 412 to 2214 of SEQ ID NO:1, or vp2 proteins produced from a nucleic acid sequence at least 70% identical to at least nucleotides 412 to 2214 of SEQ ID NO: 1 which encodes the predicted amino acid sequence of at least about amino acids 138 to 738 of SEQ ID NO:2, a heterogeneous population of AAVrh79 vp3 proteins selected from: vp3 proteins produced by expression from a nucleic acid sequence which encodes the predicted amino acid sequence of at least about amino acids 204 to 738 of SEQ ID NO:2, vp3 proteins produced from a sequence comprising at least nucleotides 610 to 2214 of SEQ ID NO: 1, or vp3 proteins produced from a nucleic acid sequence at least 70% identical to at least nucleotides 610 to 2214 of SEQ ID NO: 1 which encodes the predicted amino acid sequence of at least about amino acids 204 to 738 of SEQ ID NO:2; and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 738 of SEQ ID NO: 2, and a heterogeneous population of vp3 proteins which are the product of a nucleic acid sequence encoding at least amino acids 204 to 738 of SEQ ID NO:2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change; and (B) a vector genome in the AAVrh79 capsid, the vector genome comprising a nucleic acid molecule comprising AAV inverted terminal repeat sequences and a non-AAV nucleic acid sequence encoding a product operably linked to sequences which direct expression of the product in a host cell.

In another aspect, a method of transducing a target tissue is provided. In one embodiment, the method includes administering an AAV having an AAVrh79 capsid as described herein. In one embodiment, a method of transducing liver tissue is provided, comprising administering an AAV having the AAVrh79 capsid. In another embodiment, a method of transducing muscle tissue is provided, comprising administering an AAV having the AAVrh79 capsid.

In yet another aspect, a method of reducing deamidation of an AAVrh79 capsid is provided. In one embodiment, the method includes producing an AAVrh79 capsid from a nucleic acid sequence containing modified AAVrh79 VP codons, the nucleic acid sequence comprising independently modified glycine codons at one to four of the asparagine-glycine pairs located at position N57, N263, N385 and/or N514 in SEQ ID NO: 2, such that the modified codon encodes an amino acid other than glycine. In another embodiment, the method includes producing an AAVrh79 capsid from a nucleic acid sequence containing modified AAVrh79 vp codons, the nucleic acid sequence comprising independently modified glycine codons at one to four of the asparagine-glycine pairs located at position N94, N254, N305, N410, and/or N479 of SEQ ID NO: 2.

In a further embodiment, a rAAV8.AR2.08 is provided which comprises: (A) an AAV8.AR2.08 capsid comprising one or more of: (1) AAV8.2.08 capsid proteins comprising: a heterogeneous population of AAV8.AR2.08 vp1 proteins selected from: vp1 proteins produced by expression from a nucleic acid sequence which encodes the predicted amino acid sequence of 1 to 738 of SEQ ID NO: 18, vp1 proteins produced from SEQ ID NO: 17, or vp1 proteins produced from a nucleic acid sequence at least 70% identical to SEQ ID NO: 17 which encodes the predicted amino acid sequence of 1 to 738 of SEQ ID NO:18, a heterogeneous population of AAV8.AR2.08 vp2 proteins selected from: AAV8.AR2.08 vp2 proteins produced by expression from a nucleic acid sequence which encodes the predicted amino acid sequence of at least about amino acids 138 to 738 of SEQ ID NO: 18, vp2 proteins produced from a sequence comprising at least nucleotides 412 to 2214 of SEQ ID NO: 17, or vp2 proteins produced from a nucleic acid sequence at least 70% identical to at least nucleotides 412 to 2214 of SEQ ID NO: 17 which encodes the predicted amino acid sequence of at least about amino acids 138 to 738 of SEQ ID NO:18, a heterogeneous population of AAV8.AR2.08 vp3 proteins selected from: vp3 proteins produced by expression from a nucleic acid sequence which encodes the predicted amino acid sequence of at least about amino acids 204 to 738 of SEQ ID NO:18, vp3 proteins produced from a sequence comprising at least nucleotides 607 to 2214 of SEQ ID NO:17, or vp3 proteins produced from a nucleic acid sequence at least 70% identical to at least nucleotides 607 to 2214 of SEQ ID NO: 17 which encodes the predicted amino acid sequence of at least about amino acids 204 to 738 of SEQ ID NO: 18; and/or (2) a heterogeneous population of vp1 proteins which are the product of a nucleic acid sequence encoding the amino acid sequence of SEQ ID NO: 18, a heterogeneous population of vp2 proteins which are the product of a nucleic acid sequence encoding the amino acid sequence of at least about amino acids 138 to 738 of SEQ ID NO: 18, and a heterogeneous population of vp3 proteins which are the product of a nucleic acid sequence encoding at least amino acids 204 to 738 of SEQ ID NO: 18 wherein: the vp1, vp2 and vp3 proteins contain subpopulations with amino acid modifications comprising at least two highly deamidated asparagines (N) in asparagine-glycine pairs in SEQ ID NO: 18 and optionally further comprising subpopulations comprising other deamidated amino acids, wherein the deamidation results in an amino acid change; and (B) a vector genome in the AAV8.AR2.08 capsid, the vector genome comprising a nucleic acid molecule comprising AAV inverted terminal repeat sequences and a non-AAV nucleic acid sequence encoding a product operably linked to sequences which direct expression of the product in a host cell.

In another aspect, a method of transducing a target tissue is provided. In one embodiment, the method includes administering an AAV having an AAV8.AR2.08 capsid as described herein. In one embodiment, a method of transducing liver tissue is provided, comprising administering an AAV having the AAV8.AR2.08 capsid. In another embodiment, a method of transducing muscle tissue is provided, comprising administering an AAV having the AAV8.AR2.08 capsid.

In yet another aspect, a method of reducing deamidation of an AAV8.AR2.08 capsid is provided. In one embodiment, the method includes producing an AAV8.AR2.08 capsid from a nucleic acid sequence containing modified AAV8.AR2.08 vp codons, the nucleic acid sequence comprising independently modified glycine codons at one to four of the asparagine-glycine pairs located at position N57, N263, N385, N514, and/or N540 in SEQ ID NO: 18, such that the modified codon encodes an amino acid other than glycine. In another embodiment, the method includes producing an AAV8.AR2.08 capsid from a nucleic acid sequence containing modified AAV8.AR2.08 vp codons, the nucleic acid sequence comprising independently modified glycine codons at one to four of the asparagine-glycine pairs located at position N94, N254, N305, N521, N590, Q601, N653, and/or N665 of SEQ ID NO: 18.

In certain embodiments, a rAAV5.5.9 is provided which comprises: (A) an AAV5.5.9 capsid comprising one or more of: (1) AAVG5 capsid proteins comprising: a heterogeneous population of AAV5.5.9 vp1 proteins selected from: vp1 proteins produced by expression from a nucleic acid sequence which encodes the predicted amino acid sequence of 1 to 726 of SEQ ID NO: 10, vp1 proteins produced from SEQ ID NO:9, or vp1 proteins produced from a nucleic acid sequence at least 70% identical to SEQ ID NO:9 which encodes the predicted amino acid sequence of 1 to 726 of SEQ ID NO:1, a heterogeneous population of AAV5.5.9 vp2 proteins selected from: AAVG5 vp2 proteins produced by expression from a nucleic acid sequence which encodes the predicted amino acid sequence of at least about amino acids 137 to 726 of SEQ ID NO: 10, vp2 proteins produced from a sequence comprising at least nucleotides 409 to 2178 of SEQ ID NO:9, or vp2 proteins produced from a nucleic acid sequence at least 70% identical to at least nucleotides 409 to 2178 of SEQ ID NO:9 which encodes the predicted amino acid sequence of at least about amino acids 137 to 726 of SEQ ID NO:10, a heterogeneous population of AAV5.5.9 vp3 proteins selected from: AAV5.5.9 vp3 proteins produced by expression from a nucleic acid sequence which encodes the predicted amino acid sequence of at least about amino acids 203 to 726 of SEQ ID NO: 10, vp3 proteins produced from a sequence comprising at least nucleotides 607 to 2178 of SEQ ID NO:9, or vp3 proteins produced from a nucleic acid sequence at least 70% identical to at least nucleotides 607 to 2178 of SEQ ID NO: 9 which encodes the predicted amino acid sequence of at least about amino acids 203 to 726 of SEQ ID NO: 10; and/or (2) a heterogeneous population of vp1 proteins which are the product of a nucleic acid sequence encoding the amino acid sequence of SEQ ID NO: 10, a heterogeneous population of vp2 proteins which are the product of a nucleic acid sequence encoding the amino acid sequence of at least about amino acids 137 to 726 of SEQ ID NO: 10, and a heterogeneous population of vp3 proteins which are the product of a nucleic acid sequence encoding at least amino acids 203 to 726 of SEQ ID NO:10 wherein: the vp1, vp2 and vp3 proteins contain subpopulations with amino acid modifications comprising at least two highly deamidated asparagines (N) in asparagine-glycine pairs in SEQ ID NO: 10 and optionally further comprising subpopulations comprising other deamidated amino acids, wherein the deamidation results in an amino acid change; and (B) a vector genome in the AAV5.5.9 capsid, the vector genome comprising a nucleic acid molecule comprising AAV inverted terminal repeat sequences and a non-AAV nucleic acid sequence encoding a product operably linked to sequences which direct expression of the product in a host cell.

In another aspect, a method of transducing a target tissue is provided. In one embodiment, the method includes administering an AAV having an AAV5.5.9 capsid as described herein. In one embodiment, a method of transducing liver tissue is provided, comprising administering an AAV having the AAV5.5.9 capsid. In another embodiment, a method of transducing muscle tissue is provided, comprising administering an AAV having the AAV5.5.9 capsid.

In yet another aspect, a method of reducing deamidation of an AAV5.5.9 capsid is provided. In one embodiment, the method includes producing an AAV5.5.9 capsid from a nucleic acid sequence containing modified AAV5.5.9 vp codons, the nucleic acid sequence comprising independently modified glycine codons at one to four of the asparagine-glycine pairs located at position N57, N319, N442, and/or N502 in SEQ ID NO: 10, such that the modified codon encodes an amino acid other than glycine. In another embodiment, the method includes producing an AAV5.5.9 capsid from a nucleic acid sequence containing modified AAV5.5.9 vp codons, the nucleic acid sequence comprising independently modified glycine codons at one to four of the asparagine-glycine pairs located at position N35, N113, N204, N217, N243, N249, N293/294, N304, N399/400, N505, Q589, N618, N641, N653, N658, and/or N699 of SEQ ID NO: 10.

In another aspect, a composition comprising a mixed population of recombinant AAVrh79, AAV8.AR2.08, or AAV5.5.9, as described herein, is provided.

In yet another aspect, a recombinant AAV (rAAV) as described herein is provided, for delivering a desired gene product to a subject in need thereof.

In another aspect, a rAAV production system useful for producing a rAAV as described herein is provided. In one embodiment, the system includes (a) an AAVrh79, AAV8.AR2.08, or AAV5.5.9 capsid nucleic acid sequence encoding the predicted amino acid sequence of SEQ ID NO: 2, 10, or 18; (b) a nucleic acid molecule suitable for packaging into the AAV capsid, said nucleic acid molecule comprising at least one AAV inverted terminal repeat (ITR) and a non-AAV nucleic acid sequence encoding a gene product operably linked to sequences which direct expression of the product in a host cell; and (c) sufficient AAV rep functions and helper functions to permit packaging of the nucleic acid molecule into the recombinant AAV capsid.

These and other aspects of the invention will be apparent from the following detailed description of the invention.

### BRIEF DESCRIPTION OF THE DRAWINGS

FIG. 1A-FIG. 1G. Electrophoretic analysis of AAV8 VP isoforms. (FIG. 1A) Diagram illustrating the mechanism by which asparagine residues undergo nucleophilic attack by adjacent nitrogen atoms, forming a succinimidyl intermediate. This intermediate then undergoes hydrolysis, resolving into a mixture of aspartic acid and isoaspartic acid. The beta carbon is labeled as such. The diagram was generated in BIOVIA Draw 2018. (FIG. 1B) 1 μg of AAV8 vector was run on a denaturing one-dimensional SDS-PAGE. (FIG. 1C) Isoelectric points of carbonic anhydrase pI marker spots are shown. (FIG. 1D) 5 μg of AAV8 vector was analyzed by two-dimensional gel electrophoresis and stained with Coomassie Blue. Spots 1-20 are carbamylated carbonic anhydrase pI markers. Boxed regions are as follows: a=VP1, b=VP2, c=VP3, d=internal tropomyosin marker (arrow: tropomyosin spot of MW=33 kDa, pI=5.2). Isoelectric focusing was performed with a pI range of 4-8. FIG. 1E-FIG. 1G) Results of isoelectric focusing performed with a pI range of 4-8. 1e11 GC of wtAAV8 (FIG. 1E) or mutant (FIG. 1F and FIG. 1G) vector, which were analyzed by 2D gel electrophoresis and stained with Sypro Ruby. Protein labeling: A=VP1; B=VP2; C=VP3, D=chicken egg white conalbumin marker, E=turbonuclease marker. Isoelectric focusing was performed with a pI range of 6-10. Primary VP1/2/3 isoform spots are circled, and migration distance of major spots of markers are indicated by vertical lines (turbonuclease=dashed, conalbumin=solid).

FIG. 2A-FIG. 2E. Analysis of asparagine and glutamine deamidation in AAV8 capsid proteins. (FIG. 2A-FIG. 2B) Electrospray ionization (ESI) mass spectrometry and theoretical and observed masses of the 3+ peptide (93-103) containing Asn-94 (FIG. 2A) and Asp-94 (FIG. 2B) are shown. (FIG. 2C-FIG. 2D) ESI mass spectrometry and theoretical and observed masses of the 3+ peptide (247-259) containing Asn-254 (FIG. 2C) and Asp-254 (FIG. 2D) are shown. The observed mass shifts for Asn-94 and Asn-254 were 0.982 Da and 0.986 Da, respectively, versus a theoretical mass shift of 0.984 Da. (FIG. 2E) Percent deamidation at specific asparagine and glutamine residues of interest are shown for AAV8 tryptic peptides purified by different methods. Bars indicating deamidation at asparagine residues with N+1 glycines are crosshatched. Residues determined to be at least 2% deamidated in at least one prep analyzed were included. Data are represented as mean±standard deviation.

FIG. 3A-FIG. 3E. Structural modeling of the AAV8 VP3 monomer and analysis of deamidated sites. (FIG. 3A) The AAV8 VP3 monomer (PDB identifier: 3RA8) is shown in a coil representation. The ribbon indicates the relative degree of flexibility. Spheres indicate residues of interest. Expanded diagrams are ball and stick representations of residues of interest and their surrounding residues to demonstrate local protein structure. Underlined residues are those in NG motifs. FIG. 3B-FIG. 3E: Isoaspartic models of deamidated asparagines with N+1 glycines are shown. The 2FoFc electron density map (1 sigma level) generated from refinement of the AAV8 crystal structure (PDB ID: 3RA8) with (FIG. 3B) an asparagine model of N410 in comparison with isoaspartic acid models of (FIG. 3C) N263, (FIG. 3D) N514, and (FIG. 3E) N540. Electron density map is shown. The beta carbon is labeled as such. Arrow indicates electron density corresponding to the R group of the residue of interest.

FIG. 4A-FIG. 4D. Determination of factors influencing AAV8 capsid deamidation. An AAV8 prep was (FIG. 4A) incubated at 70° C. for three or seven days, (FIG. 4B) exposed to pH 2 or pH 10 for seven days, or (FIG. 4C) prepared for mass spectrometry using D₂O in place of H₂O to determine possible sources of deamidation not intrinsic to AAV capsid formation. (FIG. 4D) A dot blot of vector treated as in FIG. 4A using the B1 antibody (reacts to denatured capsid) and an AAV8 conformation specific antibody (reacts to intact capsids) to assess capsid structural integrity.

FIG. 5A-FIG. 5B. Deamidation frequencies in non-AAV proteins. Deamidation percentages are shown for two non-AAV recombinant proteins containing NG motifs likely to be deamidated, human carbonic anhydrase (FIG. 5A) and rat phenylalanine-hydroxylase (FIG. 5B), for comparison with AAV deamidation percentages.

FIG. 6. Comparison of AAV8 percent deamidation calculated using data analysis pipelines from two institutions. Percent deamidation at specific asparagine and glutamine residues of interest are shown for AAV8 tryptic peptides evaluated at two different institutions.

FIG. 7A-FIG. 7C illustrate functional asparagine substitutions at non-NG sites with high variability between lots. (FIG. 7A) Titers of wtAAV8 and mutant vectors were produced by small-scale triple transfection in 293 cells, as measured by quantitative PCR (qPCR). Titers are reported relative to the wtAAV8 control. Transduction efficiencies were measured as described in FIG. 8B. Titers and transduction efficiencies are normalized to the value for the wtAAV8 control. (FIG. 7B) Representative luciferase images at day 14 post-injection are shown for mice receiving wtAAV8.CB7.ffluc and N499Q capsid mutant vector. (FIG. 7C) Luciferase expression on day 14 of the study periods from C57BL/6 mice injected intravenously with wtAAV8 or mutant vectors (n=3 or 4) was measured by luciferase imaging and reported in total flux units. All data are represented as mean+standard deviation.

FIG. 8A and FIG. 8B show the results of in vitro analysis of the impact of genetic deamidation on vector performance. (FIG. 8A) Titers of wtAAV8 and genetic deamidation mutant vectors produced by small-scale triple transfection in 293 cells, as measured by quantitative PCR (qPCR). Titers are reported relative to the wtAAV8 control. NG sites with high deamidation (patterned bars), sites with low deamidation (white bars) and highly variable sites (black bars) are presented with wtAAV8 and a negative control. (FIG. 8B) Transduction efficiency of mutant AAV8 vectors producing firefly luciferase reported relative to the wtAAV8 control. Transduction efficiency is measured in luminescence units generated per GC added to HUH7 cells, and is determined by performing transductions with crude vector at multiple dilutions. Transduction efficiency data are normalized to the wt reference. All data are represented as mean±standard deviation.

FIG. 9A-FIG. 9D illustrate that vector activity loss through time is correlated to progressive deamidation. (FIG. 9A) Vector production (DNAseI resistant Genome Copies, GC) for a timecourse of triple-transfected HEK 293 cells producing AAV8 vector packaging a luciferase reporter gene. GC levels are normalized to the maximum observed value. (FIG. 9B) Purified timecourse vector was used to transduce Huh7 cells. Transduction efficiency (luminescence units per GC added to target cells) was measured as in FIG. 8B using multiple dilutions of purified timecourse vector samples. Error bars represent the standard deviation of at least 10 technical replicates for each sample time. Deamidation of AAV8 NG sites (FIG. 9C) and non-NG sites (FIG. 9D) for vector collected 1, 2 and 5 days post transfection.

FIG. 10A-FIG. 10D illustrates the impact of stabilizing asparagines on vector performance. FIG. 10A shows titers of wtAAV8 and +1 position mutant vectors produced by small-scale triple transfection in 293 cells, as measured by quantitative PCR (qPCR). Titers are reported relative to the wtAAV8 control. FIG. 10B shows the transduction efficiency of mutant AAV8 vectors producing firefly luciferase reported relative to the wtAAV8 control. Transduction efficiency was measured as in FIG. 8B using crude vector material. A two-sample t-test (*p<0.005) was run to determine significance between wtAAV8 and mutant transduction efficiency for G264A/G515A and G264A/G541A. FIG. 10C shows luciferase expression on day 14 of the study period in the liver region from C57BL/6 mice injected intravenously with wtAAV8 or mutant vectors (n=3 to 5) measured by luciferase imaging and reported in total flux units. FIG. 10D shows the titers and transduction efficiency of multi-site AAV8 mutant vectors producing firefly luciferase reported relative to the wtAAV8 control. All data are represented as mean±standard deviation.

FIG. 11A-FIG. 11C. Analysis of asparagine and glutamine deamidation in AAV9 capsid proteins. (FIG. 11A) 1e11 GCs of wtAAV9 were analyzed by 2D gel electrophoresis and stained with Sypro Ruby. Protein labeling: A=VP1; B=VP2; C=VP3, D=chicken egg white conalbumin marker, E=turbonuclease marker. Isoelectric focusing was performed with a pI range of 6-10. (FIG. 11B) Percent deamidation at specific asparagine and glutamine residues of interest are shown for AAV9 tryptic peptides purified by different methods. Bars indicating deamidation at asparagine residues with N+1 glycines are crosshatched. Residues determined to be at least 2% deamidated in at least one prep analyzed were included. Data are represented as mean±standard deviation. (FIG. 11C) Isoaspartic model of N512 is shown in the 2FoFc electron density map generated by non-biased refinement of the AAV9 crystal structure (PDB ID: 3UX1). Arrow indicates electron density corresponding to the R group of residue N512. FIG. 11D-FIG. 11F. Determination of factors influencing AAV9 capsid deamidation. (FIG. 11D) Two AAV9 preps were incubated at 70° C. for three or seven days or (FIG. 11F) exposed to pH 2 or pH 10 for seven days to determine possible sources of deamidation not intrinsic to AAV capsid formation. Data are represented as mean±standard deviation. (FIG. 11F) A dot blot of vector treated as in FIG. 11D using the B1 antibody (reacts to denatured capsid) to assess capsid structural integrity. FIG. 11G and FIG. 11H illustrate in vitro analysis of the impact of genetic deamidation on vector performance for AAV9. (FIG. 11G) Titers of wtAAV9 and genetic deamidation mutant vectors were produced by small-scale triple transfection in 293 cells, as measured by quantitative PCR (qPCR). Titers are reported relative to the wtAAV9 control. NG sites with high deamidation (patterned bars), sites with low deamidation (white bars) and highly variable sites (black bars) are presented with wtAAV8 and a negative control. (FIG. 11H) The transduction efficiency of mutant AAV9 vectors producing firefly luciferase are reported relative to the wtAAV9 control. All data are represented as mean±standard deviation. FIG. 11I-FIG. 11K show AAV9 vector in vitro potency through time. (FIG. 11I) Vector production (DNAseI resistant Genome Copies, GC) for a timecourse of triple-transfected HEK 293 cells producing AAV9 vector packaging a luciferase reporter gene. GC levels are normalized to the maximum observed value. (FIG. 11J) Crude timecourse vector was used to transduce Huh7 cells. (FIG. 11K) Transduction efficiencies of vector collected 1 day post transfection vs 5 days post transfection are shown for crude and purified vector samples. Transduction efficiency is expressed as luciferase activity/GC, normalized to the value at day 1.

FIG. 12A provides an alignment of the amino acid sequences of AAV5.5.9 [SEQ ID NO: 10] (also sometimes called AAVG5), AAV9 [SEQ ID NO: 4], and AAVPHP.B [SEQ ID NO: 12], prepared using Clustal Omega 1.2.2 and its default parameters for alignment. FIGS. 12B-12E provide an alignment of the nucleotide sequences of AAV5.5.9 [SEQ ID NO: 9], PHP.B [SEQ ID NO: 11], AAV9 [SEQ ID NO: 3], and AAVhu68 [SEQ ID NO: 14].

FIG. 13A provides an alignment of the amino acid sequences of AAV8Triple mutant (AAV8T) [SEQ ID NO: 16], AAV8.AR2.08 [SEQ ID NO: 18] (also sometimes called AAVG3 or AR2 or AAV.AR2), and AAV8 [SEQ ID NO: 20], prepared using Clustal Omega 1.2.2 and its default parameters for alignment. FIGS. 13B-13D provide an alignment of the nucleotide sequences of AAV8 Triple Mutant [SEQ ID NO: 15], AAV8. AR2.08 [SEQ ID NO: 17], AAV8 [SEQ ID NO: 19].

FIG. 14A provides an alignment of the amino acid sequences of AAVrh79 [SEQ ID NO: 2] (also sometimes called AAVG2), AAVrh10 [SEQ ID NO: 24] and AAVhu37 [SEQ ID NO: 22], using Clustal Omega 1.2.2 and its default parameters for alignment. FIGS. 14B-14D provide an alignment of the nucleotide sequences of AAVrh79 [SEQ ID NO: 1], AAVrh10 [SEQ ID NO: 22] and AAVhu37 [SEQ ID NO: 21].

FIGS. 15A and 15B illustrate the production yield for AAV8triple, AAVhu68, AAV9, AAV9 and AAVrh79 in small scale or mega scale preps of the referenced vector.

FIG. 16 provides the production purity of the mega scale preps of FIG. 15B.

FIGS. 17A to 17D show expression of luciferase in liver and muscle tissue following intramuscular (IM) administration of 3×10¹¹ GC/mouse into the gastrocnemius muscle of male C57BL/6 mice (n=5/group) using vectors expressing firefly luciferase. Expression of AVV8triple, AAVhu68, AAV9, AAV8, and AAVrh79 vectors was compared following intramuscular administration of 10¹³ GC/kg AAVrh79 into male and female cynomolgus macaques (FIG. 17E). Vectors expressing a secreted transgene (201Ig IA) were administrated intramuscularly into the gastrocnemius muscle of male RAG KO mice (n=5/group) (3×10¹⁰ or 3×10¹¹ GC/mouse) (FIG. 17F). The results indicated that AAV8triple expresses better following IM injection and at the lower dose tested the difference in expression from AAV8triple was substantial. At higher the higher dose, AVVrh79 expressed at levels at comparable to the other vectors tested.

FIG. 18A shows the plasmid used for the barcode experiments in Example 5. FIG. 18B shows the amounts of each AAV barcode variant injected into black 6 mice. The animals were sacrificed, tissue samples harvested, and DNA isolated from each of them. Total vector distribution for the three animals is shown in FIG. 18C. Actual vs. theoretical frequency of injected vector mix is shown in FIG. 18D.

FIGS. 19A-FIG. 20C show the results of the barcode biodistribution experiments of Example 5. Individual tissue samples were analyzed for individual barcode frequency in the sample vs. injection mix for genomic and cDNA. Results are shown for muscle (FIG. 19A, 19B); heart (FIG. 19C and FIG. 19D) and liver (FIG. 19E and FIG. 19F). Fold changes as compared to theoretical frequencies are shown in FIG. 20A-FIG. 20C.

FIGS. 21 and 22 show AAV8.AR2.08 biodistribution in mice as compared to AAV8. The results show that AAV8.AR2.08 is more liver specific than AAV8.

FIG. 23 compares AAV8 vs. AAV8.AR2.08 vs. AAVrh79 for titer and yield relating to manufacturability.

FIG. 24 shows AAV8.AR2.08 biodistribution in tissues (left most bar) compared with AAV8 (middle and right bars).

FIGS. 25-28 show results following administration of AAV vectors to non-human primates. FIG. 25 provides details for vectors and animals used for studies. FIG. 26 quantifies levels of GC and GFP detected in liver from animals that received AAV8, AAVrh79, or AAV8.AR2.08 vectors. FIG. 27 summarizes levels of GFP expression in HNP livers. FIG. 28 shows level of vector detected in tissue from HNP that were administered AAV8, AAVrh79, or AAV8.AR2.08 vectors.

FIG. 29 shows biodistribution of AAVrh79 vector detected in various tissues.

### DETAILED DESCRIPTION OF THE INVENTION

Provided herein are recombinant adeno-associated virus (rAAV) having sequence and charge heterogeneity in each of the three populations of capsid proteins VP1, VP2, and VP3 found within the capsid of a recombinant AAV and compositions containing same. Provided herein are novel rAAV, as well as methods for reducing the deamidation, and optionally other capsid monomer modifications. Further provided herein are modified rAAV having decreased modifications, which are useful for providing rAAV having capsids which retain greater stability, potency, and/or purity.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 of SEQ ID NO:2 may be deamidated based on the total vp1 proteins may be deamidated based on the total vp1, vp2 and vp3 proteins). Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn's

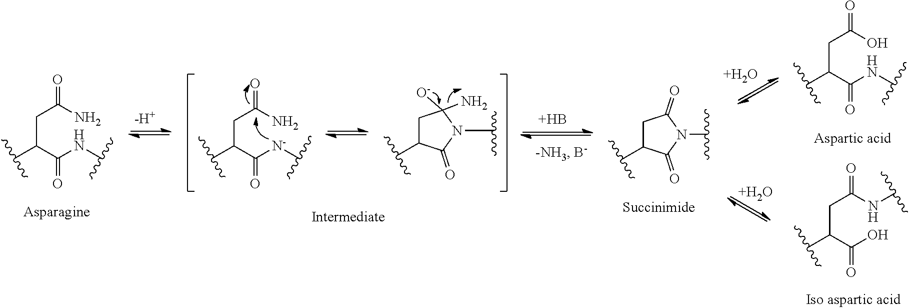

side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate e.g., as illustrated below.

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic:isoaspartic, or about 1:3 aspartic:isoaspartic, or another selected ratio.

In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

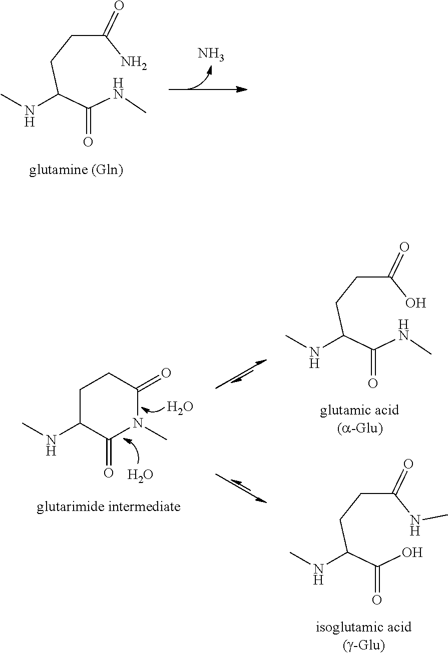

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100% or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3LC | DNA codons |
|---|---|---|---|
| Isoleucine | I | Ile | ATT, ATC, ATA |
| Leucine | L | Leu | CTT, CTC, CTA, CTG, |
| | | | TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, |
| | | | AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, |
| | | | AGA, AGG |
| Stop codons | Stop | | TAA, TAG, TGA |

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra.

Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g. a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternatively one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected. See, e.g., the tables below in which based on the numbering of AAV8, the coding sequence for at least one of the following positions: N57+1, N263+1, N385+1, N514+1, N540+1, is modified, or as shown in the tables below. In certain embodiments, AAV8 mutants avoid changing the NG pairs at positions N57, N94, N263, N305, Q467, N479, and/or N653. In certain embodiments, other AAVs avoid mutation at corresponding N positions as determined based on an alignment with AAV8, using AAV8 numbering as a reference.

These amino acid modifications may be made by conventional genetic engineering techniques. For example, a nucleic acid sequence containing modified AAV vp codons may be generated in which one to three of the codons encoding glycine in arginine-glycine pairs are modified to encode an amino acid other than glycine. In certain embodiments, a nucleic acid sequence containing modified arginine codons may be engineered at one to three of the arginine-glycine pairs, such that the modified codon encodes an amino acid other than arginine. Each modified codon may encode a different amino acid. Alternatively, one or more of the altered codons may encode the same amino acid. In certain embodiments, these modified AAVrh79, AAV8.AR2.08 or AAV5.5.9 nucleic acid sequences may be used to generate a mutant rAAV having a capsid with lower deamidation than the native AAVrh79, AAV8.AR2.08 or AAV5.5.9 capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends. e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

In certain embodiments, the mixed population of rAAV results from a production system using a single AAV capsid nucleic acid sequence encoding a predicted AAV VP1 amino acid sequence of one AAV type. However, the production and manufacture process provides the heterogenous population of capsid proteins described above.

In certain embodiments, a novel isolated AAVrh79 capsid is provided. The nucleic acid sequence encoding the AAV is provided in SEQ ID NO: 1 and the encoded amino acid sequence is provided in SEQ ID NO:2.

In certain embodiments, a rAAV comprises a AAVrh79 capsid. An AAVrh79 capsid comprises a heterogeneous population of AAVrh79 vp1 proteins, AAVrh79 vp2 proteins, and AAVrh79 vp3 proteins. In one embodiment, the AAVrh79 capsid is produced by expression from a nucleic acid sequence which encodes the predicted amino acid sequence of 1 to 738 of SEQ ID NO:2. Optionally, sequences co-expressing the vp3 protein from a nucleic acid sequence excluding the vp1-unique region (about aa 1 to 137) or the vp2-unique region (about aa 1 to 203), vp1 proteins produced from SEQ ID NO: 1, or vp1 proteins produced from a nucleic acid sequence at least 70% identical to SEQ ID NO: 1 which encodes the predicted amino acid sequence of 1 to 738 of SEQ ID NO:2. In other embodiments, the AAVrh79 vp2 proteins produced by expression from a nucleic acid sequence which encodes the predicted amino acid sequence of at least about amino acids 138 to 738 of SEQ ID NO:2, vp2 proteins produced from a sequence comprising at least nucleotides 412 to 2214 of SEQ ID NO: 1, or vp2 proteins produced from a nucleic acid sequence at least 70% identical to at least nucleotides 412 to 2214 of SEQ ID NO: 1 which encodes the predicted amino acid sequence of at least about amino acids 138 to 738 of SEQ ID NO: 2, AAVrh79 vp3 proteins produced by expression from a nucleic acid sequence which encodes the predicted amino acid sequence of at least about amino acids 204 to 738 of SEQ ID NO:2, vp3 proteins produced from a sequence comprising at least nucleotides 610 to 2214 of SEQ ID NO:1, or vp3 proteins produced from a nucleic acid sequence at least 70% identical to at least nucleotides 610 to 2214 of SEQ ID NO: 1 which encodes the predicted amino acid sequence of at least about amino acids 204 to 738 of SEQ ID NO:2.

In certain embodiments, an AAVrh79 capsid comprises: a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 738 of SEQ ID NO: 2, and a heterogeneous population of vp3 proteins which are the product of a nucleic acid sequence encoding at least amino acids 204 to 738 of SEQ ID NO: 2.

The AAVrh79 vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change. High levels of deamidation at N-G pairs N57, N263, N385 and/or N514 are observed, relative to the number of SEQ ID NO:2. Deamidation has been observed in other residues, as shown in the table below and in the examples. In certain embodiments, AAVrh79 may have other residues deamidated, e.g., typically at less than 10% and/or may have other modifications, including methylations (e.g, ˜R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g, at one or more of W248, W307, W307, M405, M437, M473, W480, W480, W505, M526, M544, M561, W621, M637, and/or W697). Optionally the W may oxidize to kynurenine.

TABLE A
AAVrh79 Deamidation

| AAVrh79 | |
|---|---|
| Deamidation based | |
| on VP1 numbering | % Deamidation |
| N57 + Deamidation | 65-90, 70-95, 80-95, 75-100, |
| | 80-100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80-95, 75-100, |
| | 80-100, or 90-100 |
| ~N410 + Deamidation | 1-25, |
| N479 + Deamidation | 1-5, 1-3 |
| ~N514 + Deamidation | 65-90, 70-95, 80-95, 75-100, |
| | 80-100, or 90-100 |
| ~Q601 + Deamidation | 0-1 |
| N653 + Deamidation | 0-2 |

In certain embodiments, an AAVrh79 capsid is modified in one or more of the positions identified in the preceding table, in the ranges provided below, as determined using mass spectrometry with a trypsin enzyme. In certain embodiments, one or more of the following positions, or the glycine following the N is modified as described herein. Residue numbers are based on the AAVrh79 sequence provided herein. See, SEQ ID NO: 2.

In certain embodiments, the nucleic acid sequence encoding the AAVrh79 vp1 capsid protein is provided in SEQ ID NO: 1. In other embodiments, a nucleic acid sequence of 70% to 99.9% identity to SEQ ID NO: 1 may be selected to express the AAVrh79 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 1. However, other nucleic acid sequences which encode the amino acid sequence of SEQ ID NO: 2 may be selected for use in producing rAAV capsids. In certain embodiments, the nucleic acid sequence has the nucleic acid sequence of SEQ ID NO: 1 or a sequence at least 70% to 99% identical, at least 75%, at least 80%, at least 85%, at least 90%, at least 95%, at least 97%, at least 99%, identical to SEQ ID NO: 1 which encodes SEQ ID NO: 2. In certain embodiments, the nucleic acid sequence has the nucleic acid sequence of SEQ ID NO: 1 or a sequence at least 70% to 99. %, at least 75%, at least 80%, at least 85%, at least 90%, at least 95%, at least 97%, at least 99%, identical to about nt 412 to about nt 2214 of SEQ ID NO: 1 which encodes the vp2 capsid protein (about aa 138 to 738) of SEQ ID NO: 2. In certain embodiments, the nucleic acid sequence has the nucleic acid sequence of about nt 610 to about nt 2214 of SEQ ID NO: 1 or a sequence at least 70% to 99. %, at least 75%, at least 80%, at least 85%, at least 90%, at least 95%, at least 97%, at least 99%, identical to nt SEQ ID NO: 1 which encodes the vp3 capsid protein (about aa 204 to 738) of SEQ ID NO: 2.

The invention also encompasses nucleic acid sequences encoding mutant AAVrh79, in which one or more residues has been altered in order to decrease deamidation, or other modifications which are identified herein. Such nucleic acid sequences can be used in production of mutant rAAVrh79 capsids.

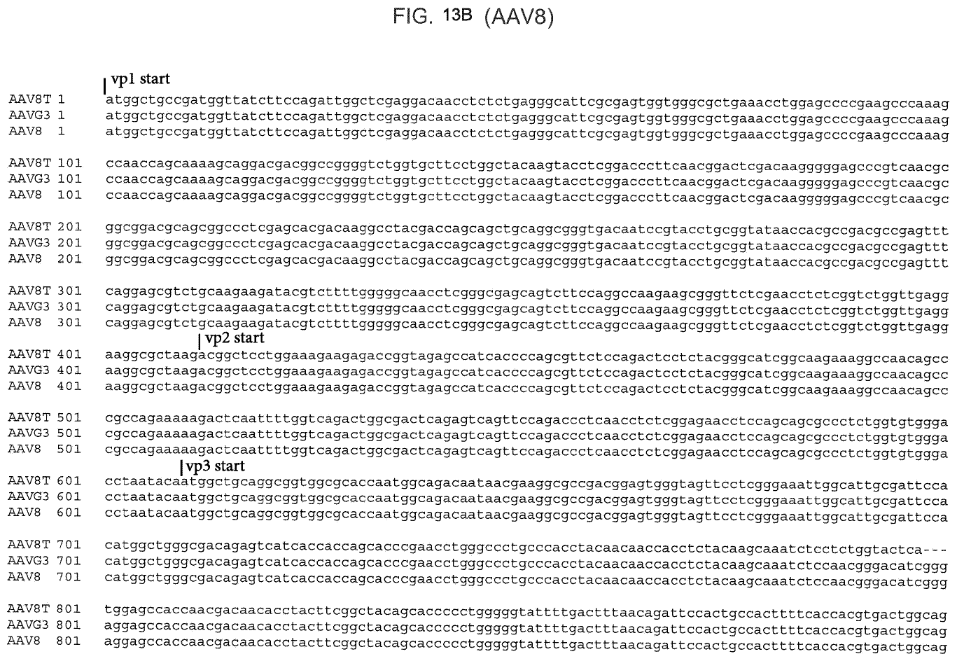

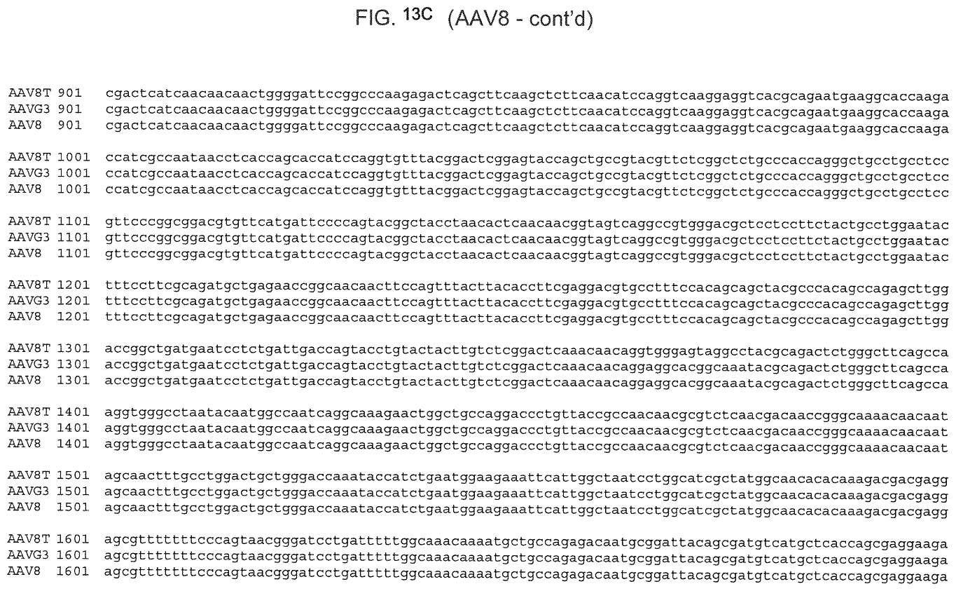

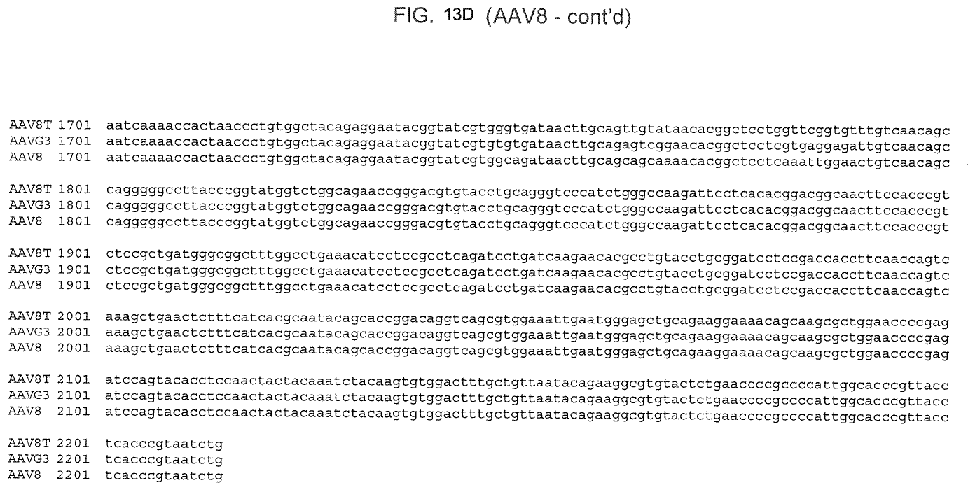

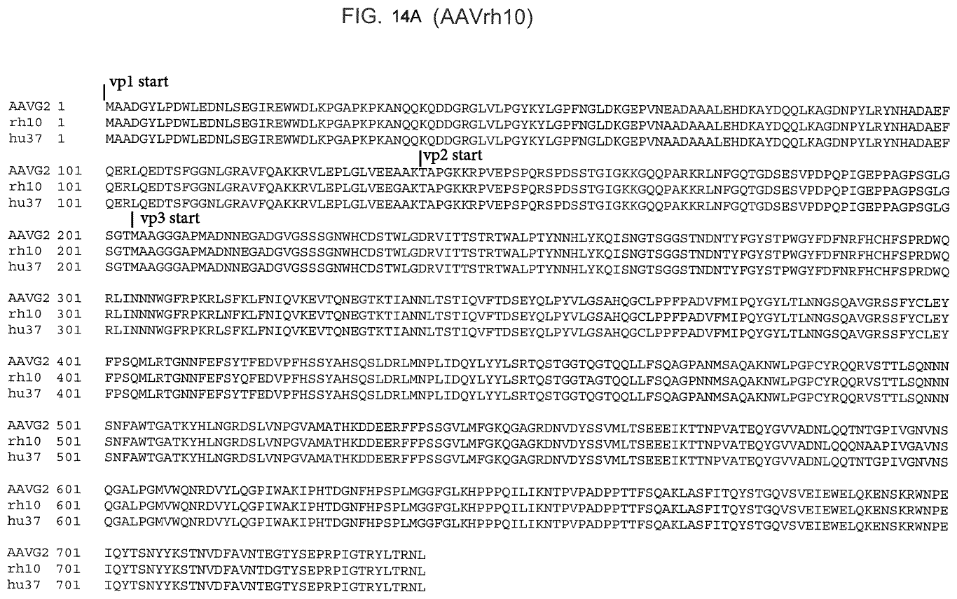

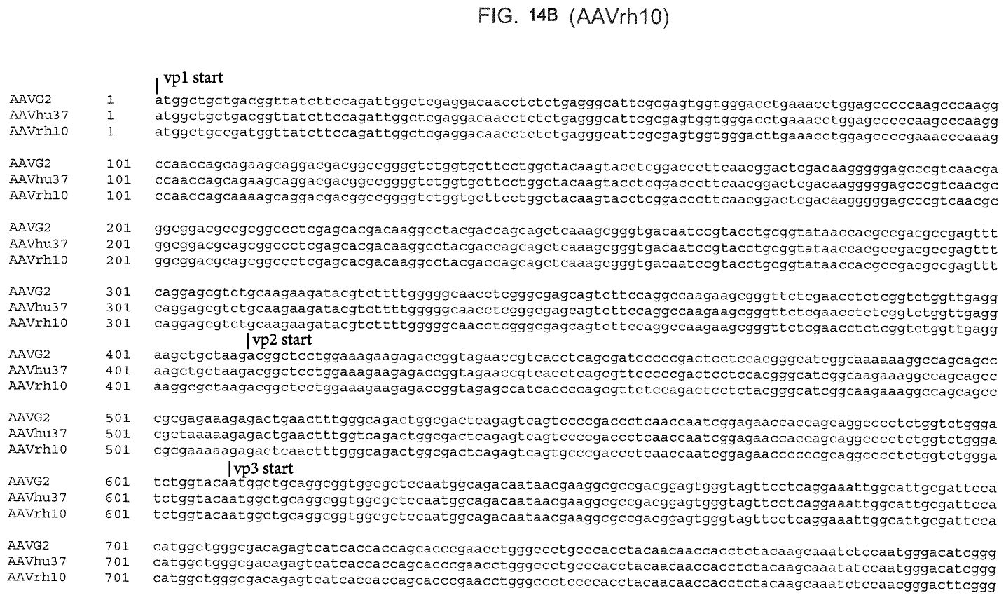

In certain embodiments, a novel AAV8.AR2.08 capsid is provided. The nucleic acid sequence encoding the AAV is provided in SEQ ID NO: 17 and the encoded amino acid sequence is provided in SEQ ID NO: 18. In one embodiment, a recombinant adeno-associated virus (rAAV) has an AAV8.AR2.08 capsid. An alignment of the amino acid sequences of AAV8T, AAV8. AR2.08 and AAV8 are provided in FIG. 13A. An alignment of the nucleic acid sequences of AAV8T, AAV8.AR2.08 and AAV8 are provided in FIG. 13B-13D.

In certain embodiments, an AAV8.AR2.08 capsid comprises AAV8.AR2.08 capsid proteins comprising: AAV8.AR2.08 vp1 proteins produced by expression from a nucleic acid sequence which encodes the predicted amino acid sequence of 1 to 738 of SEQ ID NO: 18, vp1 proteins produced from SEQ ID NO: 17, or vp1 proteins produced from a nucleic acid sequence at least 70% identical to SEQ ID NO: 17 which encodes the predicted amino acid sequence of 1 to 738 of SEQ ID NO:18, AAV8.AR2.08 vp2 proteins produced by expression from a nucleic acid sequence which encodes the predicted amino acid sequence of at least about amino acids 138 to 738 of SEQ ID NO:18, vp2 proteins produced from a sequence comprising at least nucleotides 412 to 2214 of SEQ ID NO: 17, or vp2 proteins produced from a nucleic acid sequence at least 70% identical to at least nucleotides 412 to 2214 of SEQ ID NO: 17 which encodes the predicted amino acid sequence of at least about amino acids 138 to 738 of SEQ ID NO:18, AAV8.AR2.08 vp3 proteins produced by expression from a nucleic acid sequence which encodes the predicted amino acid sequence of at least about amino acids 204 to 738 of SEQ ID NO: 18, vp3 proteins produced from a sequence comprising at least nucleotides 610 to 2214 of SEQ ID NO: 17, or vp3 proteins produced from a nucleic acid sequence at least 70% identical to at least nucleotides 610 to 2214 of SEQ ID NO: 17 which encodes the predicted amino acid sequence of at least about amino acids 204 to 738 of SEQ ID NO: 18.

Additionally, or alternatively an AAV8.AR2.08 capsid comprises: a heterogeneous population of vp1 proteins which are the product of a nucleic acid sequence encoding the amino acid sequence of SEQ ID NO: 18, a heterogeneous population of vp2 proteins which are the product of a nucleic acid sequence encoding the amino acid sequence of at least about amino acids 138 to 738 of SEQ ID NO: 18, and a heterogeneous population of vp3 proteins which are the product of a nucleic acid sequence encoding at least amino acids 204 to 738 of SEQ ID NO:18 wherein: the vp1, vp2 and vp3 proteins contain subpopulations with amino acid modifications comprising at least two highly deamidated asparagines (N) in asparagine-glycine pairs in SEQ ID NO: 18 and optionally further comprising subpopulations comprising other deamidated amino acids, wherein the deamidation results in an amino acid change. AAV8.AR2.08 is characterized by having highly deamidated residues, e.g., at positions N57, N263, N385, N514 and N540 based on the numbering of the AAV8.AR2.08 VP1 [SEQ ID NO: 18]. Additionally, residues at the positions following table and the detailed table in the application show the deamidations which have been observed in the AAV8.AR2.08 capsid.

In certain embodiments, an AAV8.AR2.08 capsid is modified in one or more of the following positions, in the ranges provided below, as determined using mass spectrometry with a trypsin enzyme. In certain embodiments, one or more of the following positions, or the glycine following the N is modified as described herein. For example, in certain embodiments, a G may be modified to an S or an A, e.g., at position 58, 264, 386, 515, or 541. Significant reduction in deamidation is observed when NG57/58 is altered to NS 57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location.

In addition to deamidation, other modifications may include isomerization (e.g, at one or more of D442 and/or D584) (1-15%), phosphorylations (e.g, at one or more of ˜S149, ˜T417, ˜T454, ˜T493, S600, and/or ˜T663), and/or oxidations (e.g., at one or more of positions ˜W22, ˜M204, ˜M212, W248, W307, M405, M437, M473, W480, W505, M526, M561, M607, ˜W609, W621, M637, W697). Still other positions may have such these or other modifications (e.g., acetylation or further deamidations).

TABLE B
AAV8.AR2.08 Deamidation

| AAV8.AR2.08 | |
|---|---|
| Deamidation based on VP1 | |
| numbering SEQ ID NO: 18 | % |
| N57 + Deamidation | 65-90, 70-95, 80-95, 75-100, |
| | 80-100, or 90-100 |
| N94 + Deamidation | 1-15 |
| ~N254 + Deamidation | 1-15 |
| ~N263 + Deamidation | 65-90, 70-95, 80-95, 75-100, |
| | 80-100, or 90-100 |
| ~N305 + Deamidation | 1-15 |
| ~N385 + Deamidation | 65-90, 70-95, 80-95, 75-100, |
| | 80-100, or 90-100 |
| ~N514 + Deamidation | 65-100 |
| ~N521 + Deamidation | 1-10 |
| ~N540 + Deamidation | 65-90, 70-95, 80-95, 75-100, |
| | 80-100, or 90-100 |
| N590 + Deamidation | 0-5 |
| Q601 + Deamidation | 0-5 |
| N653 + Deamidation | 0-5 |
| N665 + Deamidation | 0-5 |

In certain embodiments, the nucleic acid sequence encoding the AAV8.AR2.08 vp1 capsid protein is provided in SEQ ID NO: 17. In other embodiments, a nucleic acid sequence of 70% to 99.9% identity to SEQ ID NO: 17 may be selected to express the AAV8.AR2.08 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 17. However, other nucleic acid sequences which encode the amino acid sequence of SEQ ID NO: 18 may be selected for use in producing rAAV8.AR2.08 capsids. In certain embodiments, the nucleic acid sequence has the nucleic acid sequence of SEQ ID NO: 17 or a sequence at least 70% to 99% identical, at least 75%, at least 80%, at least 85%, at least 90%, at least 95%, at least 97%, at least 99%, identical to SEQ ID NO: 17 which encodes SEQ ID NO: 18. In certain embodiments, the nucleic acid sequence has the nucleic acid sequence of SEQ ID NO: 17 or a sequence at least 70% to 99. %, at least 75%, at least 80%, at least 85%, at least 90%, at least 95%, at least 97%, at least 99%, identical to about nt 412 to about nt 2214 of SEQ ID NO: 17 which encodes the vp2 capsid protein (about aa 138 to 738) of SEQ ID NO: 18. In certain embodiments, the nucleic acid sequence has the nucleic acid sequence of about nt 607 to about nt 2214 of SEQ ID NO: 17 or a sequence at least 70% to 99. %, at least 75%, at least 80%, at least 85%, at least 90%, at least 95%, at least 97%, at least 99%, identical to nt SEQ ID NO: 17 which encodes the vp3 capsid protein (about aa 204 to 738) of SEQ ID NO:18.

The invention also encompasses nucleic acid sequences encoding mutant AAV8.2.08, in which one or more residues has been altered in order to decrease deamidation, or other modifications which are identified herein. Such nucleic acid sequences can be used in production of mutant rAAV8.2.08.

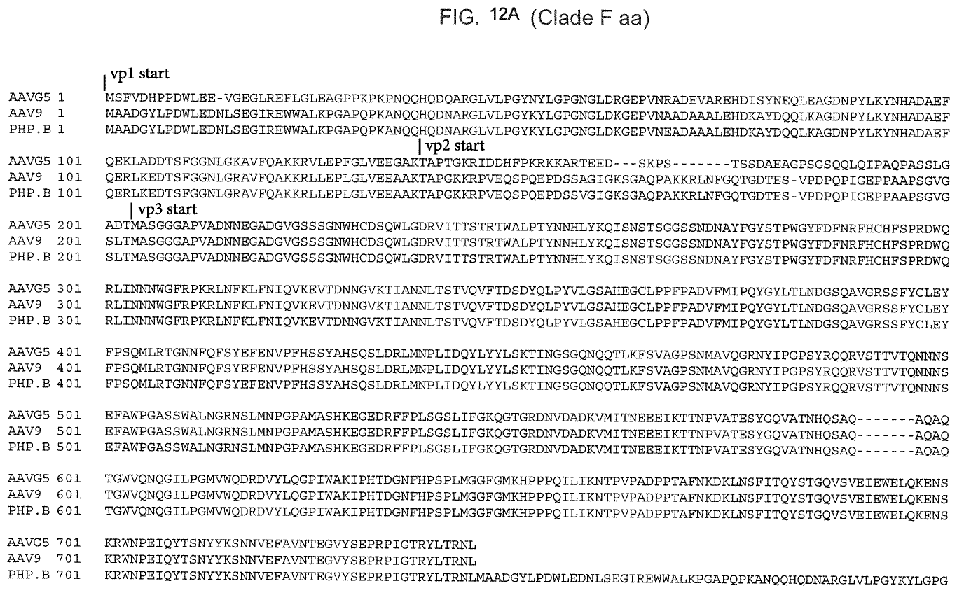

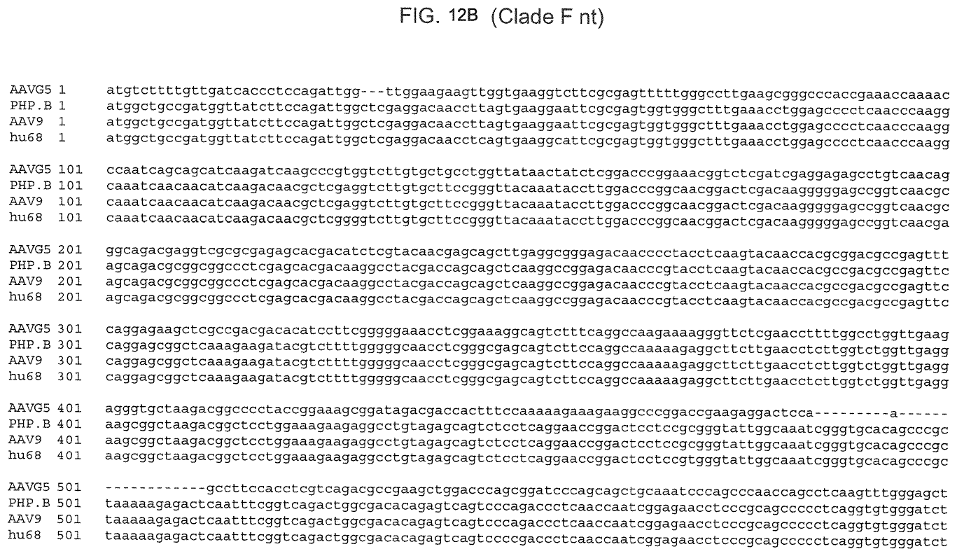

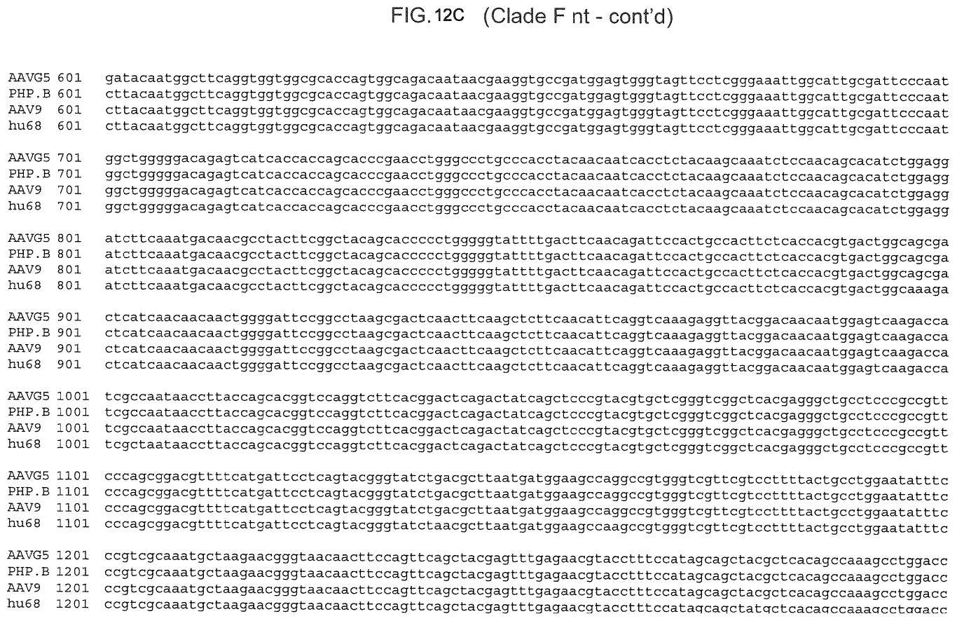

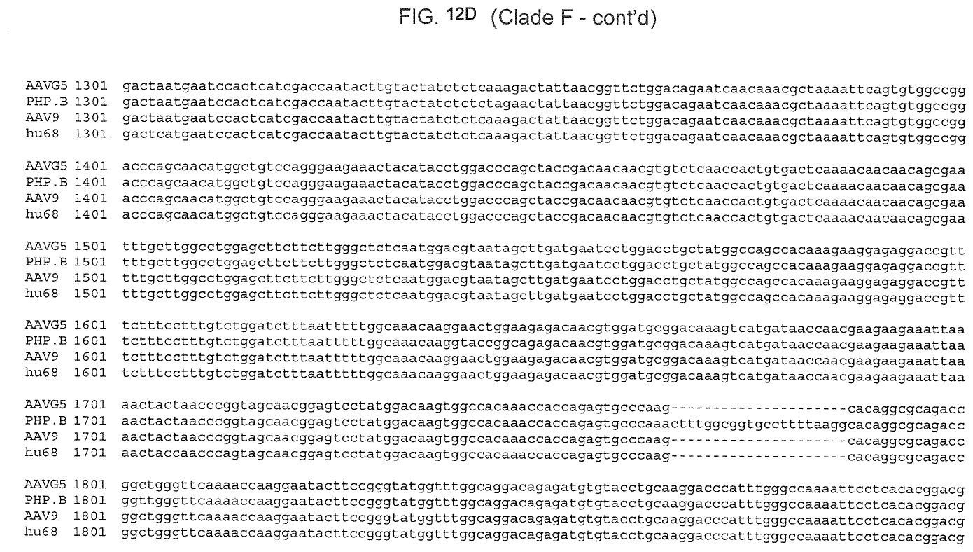

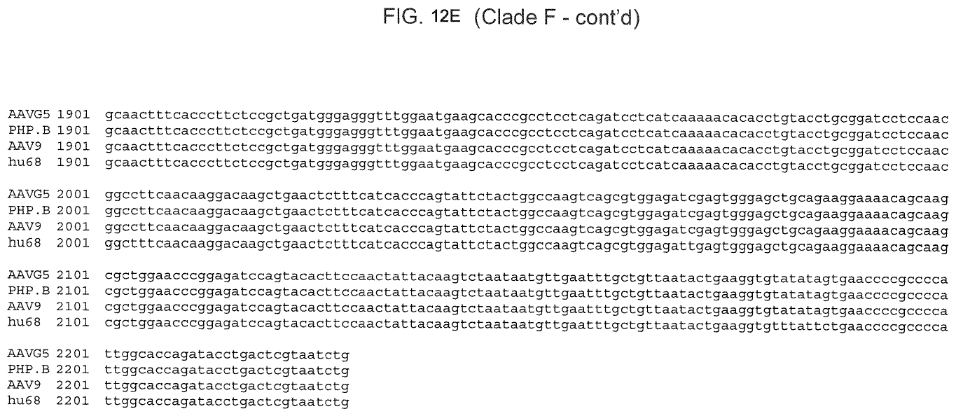

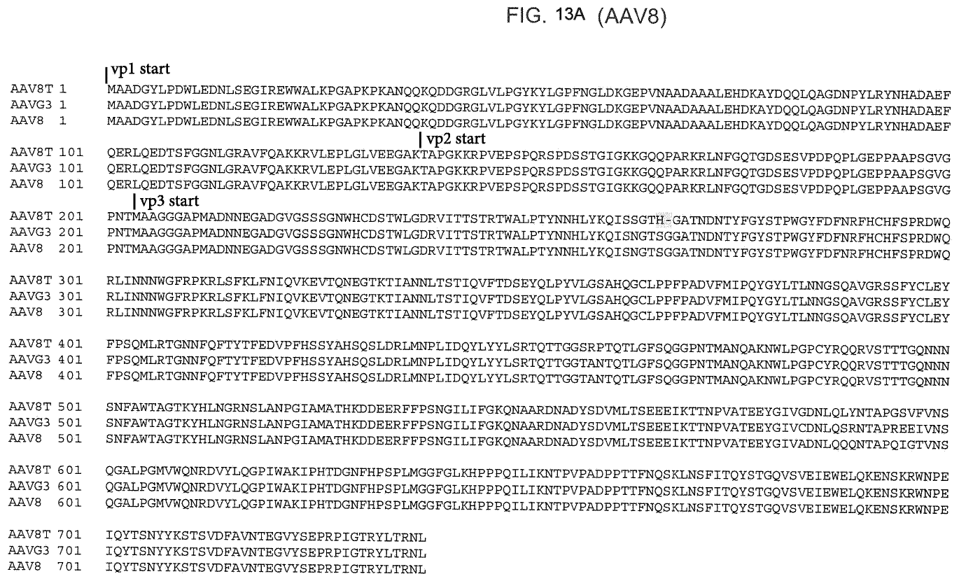

In certain embodiments, a novel AAV5.5.9 capsid is provided. The nucleic acid sequence encoding the AAV is provided in SEQ ID NO:9 and the encoded amino acid sequence is provided in SEQ ID NO: 10. An alignment of the amino acid sequences of AAV5.5.9, AAV9, and AAVPHP.B is shown in FIG. 12A. An alignment of the nucleic acid sequences of AAV5.5.9, AAV9, and AAVPHP.B is shown in FIG. 12B-12E. In one embodiment, a recombinant adeno-associated virus (rAAV) has an AAV5.5.9 capsid comprising: AAV5.5.9 capsid proteins comprising: a heterogeneous population of AAV5.5.9 vp1 proteins produced by expression from a nucleic acid sequence which encodes the predicted amino acid sequence of 1 to 726 of SEQ ID NO: 10, vp1 proteins produced from SEQ ID NO:9, or vp1 proteins produced from a nucleic acid sequence at least 70% identical to SEQ ID NO:9 which encodes the predicted amino acid sequence of 1 to 726 of SEQ ID NO: 10; a heterologous population of AAV5.5.9 vp2 proteins produced by expression from a nucleic acid sequence which encodes the predicted amino acid sequence of at least about amino acids 137 to 726 of SEQ ID NO: 10, vp2 proteins produced from a sequence comprising at least nucleotides 409 to 2178 of SEQ ID NO:9, or vp2 proteins produced from a nucleic acid sequence at least 70% identical to at least nucleotides 577 to 2178 of SEQ ID NO: 9 which encodes the predicted amino acid sequence of at least about amino acids 137 to 726 of SEQ ID NO:10, and a heterologous population of AAV5.5.9 vp3 proteins produced by expression from a nucleic acid sequence which encodes the predicted amino acid sequence of at least about amino acids 193 to 726 of SEQ ID NO:10, vp3 proteins produced from a sequence comprising at least nucleotides 577 to 2178 of SEQ ID NO:9, or vp3 proteins produced from a nucleic acid sequence at least 70% identical to at least nucleotides 577 to 2178 of SEQ ID NO:9 which encodes the predicted amino acid sequence of at least about amino acids 193 to 726 of SEQ ID NO:10.

Additionally or alternatively, an AAV5.5.9 capsid comprises: a heterogeneous population of vp1 proteins which are the product of a nucleic acid sequence encoding the amino acid sequence of SEQ ID NO: 10, a heterogeneous population of vp2 proteins which are the product of a nucleic acid sequence encoding the amino acid sequence of at least about amino acids 137 to 726 of SEQ ID NO: 10, and a heterogeneous population of vp3 proteins which are the product of a nucleic acid sequence encoding at least amino acids 193 to 726 of SEQ ID NO:10 wherein: the vp1, vp2 and vp3 proteins contain subpopulations with amino acid modifications comprising at least two highly deamidated asparagines (N) in asparagine-glycine pairs in SEQ ID NO: 10 and optionally further comprising subpopulations comprising other deamidated amino acids, wherein the deamidation results in an amino acid change.

TABLE C
AAV5.5.9 Deamidation

| AAV5.5.9 | |
|---|---|
| Deamidation based on VP1 | |
| numbering SEQ ID NO: 10 | % |
| N35 + Deamidation | 0-15, 1-10 |
| ~N57 + Deamidation | 65-90, 70-95, 80-95, 75-100, |
| | 80-100, or 90-100 |
| N113 + Deamidation | 0-15 |
| ~N204 + Deamidation | 0-20, 1-20 |
| N217 + Deamidation | 0-5, 1-5 |
| ~N243 + Deamidation | 0-25, 1-25 |
| Q249 + Deamidation | 1-20 |
| N293/294 + Deamidation | 10-45, 15-40 |
| N304 + Deamidation | 1-10 |
| N309 + Deamidation | 1-2 |
| Q311 + Deamidation | 1-2 |
| ~N319 + Deamidation | 65-90, 70-95, 80-95, 75-100, |
| | 80-100, or 90-100 |
| N399/400 + Deamidation | 5-40, 10-40, 15-35 |
| ~N442 + Deamidation | 65-90, 70-95, 80-95, 75-100, |
| | 80-100, or 90-100 |
| N467 + Deamidation | 1-5 |
| N502 + Deamidation | 65-90, 70-95, 80-95, 75-100, |
| | 80-100, or 90-100 |
| N505 + Deamidation | 5-25, 10-25 |
| ~Q589 + Deamidation | 5-30, 10-30 |
| N618 + Deamidation | 1-15, 5-10 |
| ~N641 + Deamidation | 1-15, 5-10 |
| N653 + Deamidation | 1-15, 5-10 |
| ~N658 + Deamidation | 5-40, 10-30 |
| N694 + Deamidation | 0-5 |
| ~N699 + Deamidation | 1-10 |

In certain embodiments, the nucleic acid sequence encoding the AAV5.5.9 vp1 capsid protein is provided in SEQ ID NO: 9. In other embodiments, a nucleic acid sequence of 70% to 99.9% identity to SEQ ID NO: 9 may be selected to express the AAV5.5.9 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 9. However, other nucleic acid sequences which encode the amino acid sequence of SEQ ID NO: 10 may be selected for use in producing rAAV5.5.9 capsids. In certain embodiments, the nucleic acid sequence has the nucleic acid sequence of SEQ ID NO: 10 or a sequence at least 70% to 99% identical, at least 75%, at least 80%, at least 85%, at least 90%, at least 95%, at least 97%, at least 99%, identical to SEQ ID NO: 9 which encodes SEQ ID NO: 10. In certain embodiments, the nucleic acid sequence has the nucleic acid sequence of SEQ ID NO: 9 or a sequence at least 70% to 99. %, at least 75%, at least 80%, at least 85%, at least 90%, at least 95%, at least 97%, at least 99%, identical to about nt 409 to about nt 2178 of SEQ ID NO: 9 which encodes the vp2 capsid protein (about aa 137 to 726) of SEQ ID NO: 10. In certain embodiments, the nucleic acid sequence has the nucleic acid sequence of about nt 577 to about nt 2178 of SEQ ID NO:9 or a sequence at least 70% to 99%, at least 75%, at least 80%, at least 85%, at least 90%, at least 95%, at least 97%, at least 99%, identical to nt 577 to 2178 SEQ ID NO: 9 which encodes the vp3 capsid protein (about aa 193 to 726) of SEQ ID NO: 10.

The invention also encompasses nucleic acid sequences encoding mutant AAV5.5.9, in which one or more residues has been altered in order to decrease deamidation, or other modifications which are identified herein. Such nucleic acid sequences can be used in production of mutant rAAV5.5.9.

I. rAAV Vectors

As indicated above, the novel AAV sequences and proteins are useful in production of rAAV and are also useful in recombinant AAV vectors which may be antisense delivery vectors, gene therapy vectors, or vaccine vectors. Additionally, the engineered AAV capsids described herein may be used to engineer rAAV vectors for delivery of a number of suitable nucleic acid molecules to target cells and tissues.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterogeneous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid, resulting in a pseudotyped vector. In one embodiment, the ITR sequences from AAV2. A shortened version of the 5′ ITR, termed ΔITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see. e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polyomavirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In one embodiment, the promoter is a liver specific promoter, such as that termed LSP exemplified herein.

In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable poly A sequences include, e.g., SV40, SV50, bovine growth hormone (bGH), human growth hormone, and synthetic poly As. Optionally, one or more sequences may be selected to stabilize mRNA. An example of such a sequence is a modified WPRE sequence, which may be engineered upstream of the poly A sequence and downstream of the coding sequence [see, e.g., MA Zanta-Boussif, et al, Gene Therapy (2009) 16:605-619.

These rAAVs are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

### Therapeutic Transgenes

Useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In most embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic demential), among others. See, e.g., www_orpha_net/consor/cgi-bin/Disease_Search_List_php; rarediseases_info_nih_gov/diseases.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregulin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1a, IL-1B, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SPI, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1, and other suitable gene editing constructs.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase, associated with phenylketonuria (PKU); branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); Niemann-Pick disease, type C1); propionic academia (PA); low density lipoprotein receptor (LDLR) protein, associated with familial hypercholesterolemia (FH); UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases: thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Additional genes and diseases of interest include, e.g., dystonin gene related diseases such as Hereditary Sensory and Autonomic Neuropathy Type VI (the DST gene encodes dystonin; dual AAV vectors may be required due to the size of the protein (˜7570 aa); SCN9A related diseases, in which loss of function mutants cause inability to feel pain and gain of function mutants cause pain conditions, such as erythromelagia. Another condition is Charcot-Marie-Tooth type 1F and 2E due to mutations in the NEFL gene (neurofilament light chain). characterized by a progressive peripheral motor and sensory neuropathy with variable clinical and electrophysiologic expression.

In certain embodiments, the rAAV described herein may be used in treatment of mucopolysaccaridoses (MPS) disorders. Such rAAV may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome).

Immunogenic Transgenes In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKRIC2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAVI, CBF8, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1r, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMBI, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQO1, NRID1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RABSA, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLCIA4, SLC20A1, SMO, sphingomyelin phosphodiesterase 1 (SMPD1), SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFB1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9. A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the invention: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-| 46a*, hsa-miR-|46b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-24, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548 m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. For example, miRNA targeting chromosome 8 open reading frame 72 (C9orf72) which expresses superoxide dismutase (SOD1), associated with amyotrophic lateral sclerosis (ALS) may be of interest.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex.

Still other useful transgenes may include those encoding immunoglobulins which confer passive immunity to a pathogen. An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The anti-pathogen construct is selected based on the causative agent (pathogen) for the disease against which protection is sought. These pathogens may be of viral, bacterial, or fungal origin, and may be used to prevent infection in humans against human disease, or in non-human mammals or other animals to prevent veterinary disease.

The rAAV may include genes encoding antibodies, and particularly neutralizing antibodies against a viral pathogen. Such anti-viral antibodies may include anti-influenza antibodies directed against one or more of Influenza A, Influenza B, and Influenza C. The type A viruses are the most virulent human pathogens. The serotypes of influenza A which have been associated with pandemics include, HINI, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Other target pathogenic viruses include, arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutination encephalomyelitis virus (pig), feline infectious peritonitis virus (cat), feline enteric coronavirus (cat), canine coronavirus (dog). The human respiratory coronaviruses have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleukopenia virus, canine parvovirus, and porcine parvovirus. The adenovirus family includes viruses (EX, AD7, ARD, O.B.) which cause respiratory disease. Thus, in certain embodiments, a rAAV vector as described herein may be engineered to express an anti-ebola antibody, e.g., 2G4, 4G7, 13C6, an anti-influenza antibody, e.g., FI6, CR8033, and anti-RSV antibody, e.g, palivizumab, motavizumab. A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The rAAV may include genes encoding antibodies, and particularly neutralizing antibodies against a bacterial pathogen such as the causative agent of anthrax, a toxin produced by Bacillus anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2:5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

The rAAV may include genes encoding antibodies, and particularly neutralizing antibodies, against pathogenic factors of diseases such as Alzheimer's disease (AD), Parkinson's disease (PD), GBA-associated-Parkinson's disease (GBA-PD), Rheumatoid arthritis (RA), Irritable bowel syndrome (IBS), chronic obstructive pulmonary disease (COPD), cancers, tumors, systemic sclerosis, asthma and other diseases. Such antibodies may be, without limitation, e.g., alpha-synuclein, anti-vascular endothelial growth factor (VEGF) (anti-VEGF), anti-VEGFA, anti-PD-1, anti-PDL1, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-1beta, anti-epidermal growth factor receptor, anti-CD25, anti-RANK ligand, anti-complement system protein C5, anti-CD11a, anti-CD3 receptor, anti-alpha-4 (α4) integrin, anti-RSV F protein, and anti-integrin α₄β₇. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-beta-amyloid fibril, anti-beta-amyloid plaques, anti-tau, a bapineuzumab, among others. Other suitable antibodies for treating a variety of indications include those described, e.g., in PCT/US2016/058968, filed 27 Oct. 2016, published as WO 2017/075119A1.

II. rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the transgene construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the vector genome including the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Publication No. WO 2017/160360, which is incorporated by reference herein. Purification methods for AAV8 Publication No. WO2017/100676, and rh10, International Patent Publication No. WO 2017/100704 and for AAV1, International Patent Publication No. WO 2017/100674, are all incorporated by reference herein.

To calculate empty and full particle content, VP3 band volumes for a selected sample (e.g., in examples herein an iodixanol gradient-purified preparation where #of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hum Gene Ther Methods. 2014 April; 25 (2): 115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1)] which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### III. Compositions and Uses

Provided herein are compositions containing at least one rAAV stock (e.g., an rAAV stock or a mutant rAAV stock) and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney), oral, inhalation, intranasal, intrathecal, intratracheal, intraarterial, direct delivery to the eye (optionally via ocular delivery, subretinal injection, intra-retinal injection, intravitreal, topical), intravenous, intramuscular, subcutaneous, intradermal, and other parental routes of administration. In one embodiment, the route of administration is subretinal or intravitreal injection. Routes of administration may be combined, if desired.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass.

In another aspect, an aqueous suspension suitable for administration to a subject is provided. In one embodiment, the suspension includes an aqueous suspending liquid and about 1×10⁹ viral particles to about 1×10¹³ GC or viral particles per eye of a recombinant adeno-associated virus (rAAV) as described herein useful as a therapeutic for the treatment or prevention of ocular diseases. In one embodiment, the suspension is suitable for subretinal or intravitreal injection.

In one embodiment, the viral constructs may be delivered in doses of from at least about least 1×10⁹ GCs to about 1×10¹⁵ GC, or about 1×10¹¹ GC to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous, subretinal or intravitreal delivery, a pH of about 6.8 to about 7.2 may be desired. However, other pHs within the broadest ranges and these subranges may be selected for other route of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In one example, the formulation may contain, e.g., buffered saline solution comprising one or more of sodium chloride, sodium bicarbonate, dextrose, magnesium sulfate (e.g., magnesium sulfate·7H₂O), potassium chloride, calcium chloride (e.g., calcium chloride·2H₂O), dibasic sodium phosphate, and mixtures thereof, in water. Suitably, for intrathecal delivery, the osmolarity is within a range compatible with cerebrospinal fluid (e.g., about 275 to about 290); see, e.g., emedicine_medscape_com/article/2093316-overview. Optionally, for intrathecal delivery, a commercially available diluent may be used as a suspending agent, or in combination with another suspending agent and other optional excipients. See, e.g., Elliotts B® solution [Lukare Medical]. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In one aspect, the vectors provided herein may be administered intrathecally via the method and/or the device. See, e.g., WO 2017/181113, which is incorporated by reference herein. Alternatively, other devices and methods may be selected. The method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient's cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient.

This method and this device may each optionally be used for intrathecal delivery of the compositions provided herein. Alternatively, other methods and devices may be used for such intrathecal delivery.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (+10%) from the reference given, unless otherwise specified.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199 (3): p. 381-390, which is incorporated by reference herein.

As used herein, an “expression cassette” refers to a nucleic acid molecule which comprises a coding sequence, promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the coding sequence for the gene product described herein flanked by packaging signals of the viral genome and other expression control sequences such as those described herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

The term “translation” in the context of the present invention relates to a process at the ribosome, wherein an mRNA strand controls the assembly of an amino acid sequence to generate a protein or a peptide.

As used throughout this specification and the claims, the terms “comprising” and “including” are inclusive of other components, elements, integers, steps and the like. Conversely, the term “consisting” and its variants are exclusive of other components, elements, integers, steps and the like.

As described above, the term “about” when used to modify a numerical value means a variation of +10%, unless otherwise specified.

The following examples are illustrative only and are not intended to limit the present invention.

### EXAMPLES

The following examples report the extensive deamidation of AAV8 and additional diverse AAV serotypes, with supporting evidence from structural, biochemical, and mass spectrometry approaches. The extent of deamidation at each site was dependent on the age of the vector and multiple primary-sequence and 3D-structural factors, but was largely independent of the conditions of vector recovery and purification. We demonstrate the potential for deamidation to impact vector transduction activity, and correlate an early timepoint loss in vector activity to rapidly progressing, spontaneous deamidation at several AAV8 asparagines. We explore mutational strategies that stabilize side-chain amides, improving vector transduction and reducing the lot-to-lot molecular variability that is a key concern in biologics manufacturing. This study illustrates a previously unknown aspect of AAV capsid heterogeneity and highlights its importance in the development of these vectors for gene therapy.

In Example 1 the characterization of post-translational modifications to the AAV8 vector capsid by one- and two-dimensional gel electrophoresis, mass spectrometry, and de novo structural modeling. Following the identification of a number of putative deamidation sites on the capsid surface, we evaluate their impact on capsid structure and function both in vitro and in vivo. Example 1 further extends this analysis to AAV9 to determine if this phenomenon applies to serotypes other than AAV8, confirming that deamidation of the AAV capsid is not serotype specific. Examples 2-5 show deamidation in distinct AAVs.

### Example 1: Deamidation of Amino Acids on the Surface of Adeno-Associated Virus Capsids

### A. Materials and Methods

### 1 1D and 2D Gel Electrophoresis

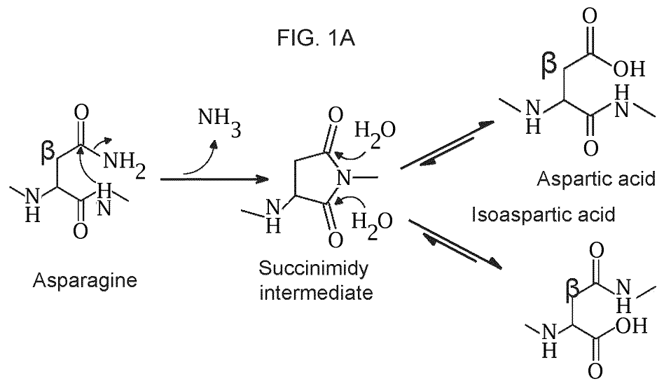

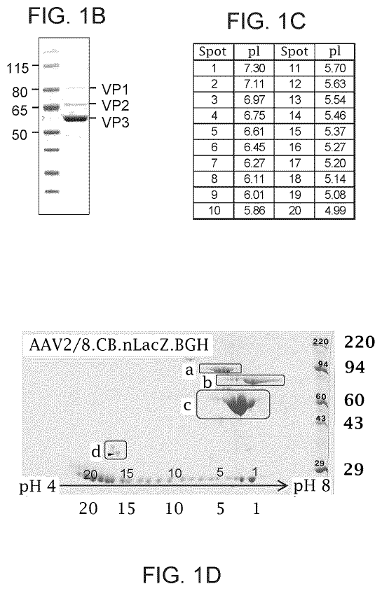

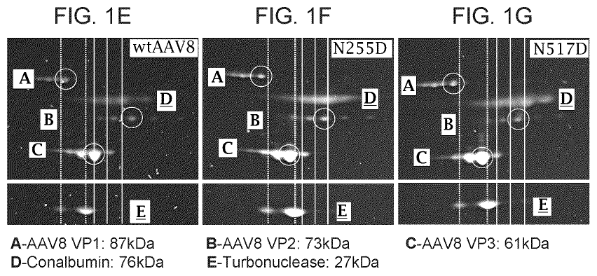

For 1D SDS polyacrylamide gel electrophoresis (SDS-PAGE) analysis, we first denatured AAV vectors at 80° C. for 20 minutes in the presence of lithium dodecyl sulfate and reducing agent. Then, we ran them on a 4-12% Bis-Tris gel for 90 minutes at 200V and stained with Coomassie blue. For the data in FIG. 1A-FIG. 1D, Kendrick Laboratories, Inc. (Madison, WI) performed the 2D gel electrophoresis. For subsequent experiments, we performed 2D SDS-PAGE in-house. For this, we combined 3×10¹¹ GCs of AAV vector with 500 U turbonuclease marker (Accelagen, San Diego, CA) in 150 μL phosphate buffered saline (PBS) with 35 mM NaCl and 1 mM MgCl2 and incubated at 37° C. for ten minutes. We next added nine volumes of absolute ethanol, vortexed the samples, and incubated them at −80° C. for at least two hours followed by incubation on ice for five minutes and centrifugation at maximum speed for 30 minutes at 15° C. We decanted the supernatant and air-dried the pellet, which we then resuspended in resuspension buffer #1 [0.15% SDS, 50 mM dithiothreitol (DTT), 10 mM Tris pH 7.5, and 1 μL pH6-9 ampholytes, ThermoFisher ZM0023, added day-of, in ddH₂O] and incubated undisturbed at room temperature. After 30 minutes, we flicked the sample tubes to mix them, added 1 μg chicken conalbumin marker (Sigma Aldrich, St. Louis, MO), and incubated the samples at 37° C. for 30 minutes, flicking to mix at 15 minutes. Samples were then transferred to 50° C. for 15-20 minutes, vortexed, incubated at 95° C. for 2.5 minutes, and allowed to cool before being centrifuged at maximum speed for one minute and briefly vortexed. We then mixed 10 μL of each sample with 140 μL resuspension buffer #2 (9.7M urea, 2% CHAPS, 0.002% bromophenol blue, and 0.05% ampholytes, described above, added day-of, in ddH₂O) and incubated at room temperature for ten minutes. We then applied the mixtures to pH 6-10 immobilized pH gradient (IPG) strips (ThermoFisher Waltham, MA) and ran them on the ZOOM IPGRunner system according to manufacturer's instructions. We used the following isoelectric focusing parameters: 100-1,000V for 120 minutes, 1,000-2,000V for 120 minutes, 2,000V for 120 minutes, limits of 0.1 W and 0.05 mA per strip run. IPG strips were then reduced and loaded in a single-well 4-12% Bis-Tris gel and run in 1D as described above. We determined the relative migration of AAV VPs by comparison to internal control proteins turbonuclease (Accelagen, 27 kDa) and chicken egg white conalbumin (Sigma Aldrich, 76 kDa, pI 6.0-6.6).

### 2. Vector Production

The University of Pennsylvania Vector Core produced recombinant AAV vectors for 1D and 2D gel electrophoresis and mass spectrometry experiments and purified them by cesium chloride or iodixanol gradients as previously described. (Lock M, et al. Hum Gene Ther 2010; 21 (10): 1259-71; Gao G P, et al. Proc Natl Acad Sci USA. 2002; 99 (18): 11854-9). We produced the affinity purified vectors as follows: We grew HEK293 cells in ten 36-layer hyperstack vessels (Corning), co-transfected them with a mixture of vector genome plasmid (pAAV-LSP-IVS2.hFIXco-WPRE-bGH), trans plasmid containing AAV2 rep and AAV8 cap genes, and adenovirus helper plasmid. We used PEIpro (PolyPlus) as the transfection reagent. Five days post transfection, the supernatant was harvested, clarified through Sartoguard PES Midicap filters (Sartorious Stedim), and treated with benzonase (Millipore), after which we added salt to bring it to 0.6M. The clarified bulk harvest material was concentrated ten-fold by tangential flow filtration (TFF) and then diafiltered against four volumes of affinity column loading buffer. We captured vectors on a POROS CaptureSelect (ThermoFisher) affinity column and eluted the vector peak at low pH directly into neutralization buffer. We diluted the neutralized eluate into a high-pH binding buffer and loaded it onto an anion exchange polishing column (Cimultus QA-8; Bia Separations), where the preparation was enriched for genome-containing (full) particles. The full vector particles were eluted with a shallow salt elution gradient and neutralized immediately. Finally, we subjected the vector to a second round of TFF for final concentration and buffer exchange into formulation buffer (PBS+0.001% pluronic F-68).

We produced mutant vectors for in vitro assays by small-scale triple transfection of HEK293 cells in six-well plates. We mixed 5.6 μL of a 1 mg/mL polyethylenimine solution in 90 μL serum-free media with plasmid DNA (0.091 μg cis plasmid, 0.91 μg trans plasmid, 1.82 μg deltaF6 Ad-helper plasmid, in 90 μL serum-free media), incubated it at room temperature for 15 minutes, and added it to cells in and additional 0.8 mL of fresh serum-free media. The next day, we replaced 0.5 mL of the top media with full serum media. We harvested vector three days post-transfection by three freeze/thaw cycles followed by centrifugation to remove cell debris and supernatant harvest. Cis plasmid contained a transgene cassette encoding the firefly luciferase transgene under the control of the chicken-beta actin (CB7) promoter with the Promega chimeric intron and rabbit beta-globin (RBG) polyadenylation signal. Trans plasmid encoded the wtAAV8 cap gene; to generate mutant AAV8 cap variants, we used the Quikchange Lightning Mutagenesis kit (Agilent Technologies, Wilmington, DE). Vector was titered as previously described. (Lock M, et al. Hum Gene Ther 2010; 21 (10): 1259-71).

For timecourse vector production experiments, we generated vector by medium-scale triple transfection of HEK293 cells in 15 cm tissue culture dishes. Per plate, we mixed 36 μL of a 1 mg/mL polyethylenimine solution in 2 mL serum-free media with plasmid DNA (0.6 μg cis plasmid, 5.8 μg trans plasmid, 11.6 μg deltaF6 Ad-helper plasmid), incubated it at room temperature for 15 minutes, and added it to cells at approximately 60% confluency on plates refreshed with 14 ml of serum-free media. The following day, we replaced 8 ml of the top media with fresh, full serum media. We harvested vector by collecting all top media, scraping cells from the dish and freezing this at −80° C. We recovered crude vector from the supernatant/cell mixture by applying 3 freeze/thaw cycles, and clarifying the lysate by centrifugation. We purified and concentrated the vector for mass spectrometry analysis by adding benzonase, 1M Tris pH7.5, and 5M NaCl to the clarified lysate to final concentrations of 20 mM Tris and 360 mM NaCl. We captured vectors on a 1 ml POROS CaptureSelect affinity column and eluted the vector peak at low pH directly into neutralization buffer. Fractions were analysed by absorbance at 280 nm, and the most concentrated fraction was subjected to mass spectrometry analysis

For in vivo experiments, we produced vectors as previously described with a wtAAV8 capsid or with one of the 6 deamidation mutants; the transgene cassette included a CB7 promoter, PI intron, firefly luciferase transgene, and RBG polyadenylation signal (Lock M, et al. Hum Gene Ther 2010; 21 (10): 1259-71).

### 3. Mass Spec Run/Digest/Analysis

Materials: We purchased ammonium bicarbonate, DTT, iodoacetamide (IAM), and 180-enriched water (97.1% purity) from Sigma (St. Louis, MO); and acetonitrile, formic acid, trifluoroacetic acid (TFA), 8M guanidine hydrochloride (GndHCl), and trypsin from Thermo Fischer Scientific (Rockford, IL).

Trypsin digestion: We prepared stock solutions of 1M DTT and 1.0M iodoacetamide. Capsid proteins were denatured and reduced at 90° C. for ten minutes in the presence of 10 mM DTT and 2M GndHCl. We allowed the samples to cool to room temperature and then alkylated them with 30 mM IAM at room temperature for 30 minutes in the dark. We quenched the alkylation reaction with the addition of 1 mL DTT. We added 20 mM ammonium bicarbonate (pH 7.5-8) to the denatured protein solution at a volume that diluted the final GndHCl concentration to 200 mM. We added trypsin solution for a 1:20 trypsin to protein ratio and incubated at 37° C. overnight. After digestion, we added TFA to a final concentration of 0.5% to quench the digestion reaction.

For 18O-water experiments, the capsid sample was first buffer exchanged into 100 mM ammonium bicarbonate prepared in 180-water using Zeba spin desalting columns (Thermo Scientific, Rockford, IL). To ensure a complete removal of the water in the sample, we performed the buffer exchange twice. We prepared stock solutions of 1M DTT and 1M IAM in 180-water. We followed the same denaturation, alkylation, and digestion steps as above with 180-water reagents and buffers.

Liquid chromatography tandem-mass spectrometry: We performed online chromatography with an Acclaim PepMap column (15 cm long, 300 μm inner diameter) and a Thermo UltiMate 3000 RSLC system (Thermo Fisher Scientific) coupled to a Q Exactive HF with a NanoFlex source (Thermo Fisher Scientific). During online analysis, the column temperature was maintained at a temperature of 35° C. We separated peptides with a gradient of mobile phase A (MilliQ water with 0.1% formic acid) and mobile phase B (acetonitrile with 0.1% formic acid). We ran the gradient from 4% B to 6% B over 15 minutes, to 10% B for 25 minutes (40 minutes total), and then to 30% B for 46 minutes (86 minutes total). We loaded the samples directly to the column. The column size was 75 cm×15 um I.D. and was packed with 2 micron C18 media (Acclaim PepMap). Due to the loading, lead-in, and washing steps, the total time for each liquid chromatography tandem-mass spectrometry run was about two hours.

We acquired mass spectrometry data using a data-dependent top-20 method on the Q Exactive HF mass spectrometer, dynamically choosing the most abundant not-yet-sequenced precursor ions from the survey scans (200-2000 m/z). We performed sequencing via higher energy collisional dissociation fragmentation with a target value of 1e5 ions determined with predictive automatic gain control; we performed isolation of precursors with a window of 4 m/z. We acquired survey scans at a resolution of 120,000 at 200 m/z. We set the resolution for HCD spectra to 30,000 at m/z200 with a maximum ion injection time of 50 ms and a normalized collision energy of 30. We set the S-lens RF level to 50, which gave optimal transmission of the m/z region occupied by the peptides from our digest. We excluded precursor ions with single, unassigned, or six and higher charge states from fragmentation selection.

Data processing: We used BioPharma Finder 1.0 software (Thermo Fischer Scientific) to analyze all data acquired. For peptide mapping, we performed searches using a single-entry protein FASTA database with carbamidomethylation set as a fixed modification, and oxidation, deamidation, and phosphorylation set as variable modifications. We used a 10 ppm mass accuracy, a high protease specificity, and a confidence level of 0.8 for tandem-mass spectrometry spectra. Mass spectrometric identification of deamidated peptides is relatively straightforward, as deamidation adds to the mass of intact molecule +0.984 Da (the mass difference between —OH and —NH₂ groups). We determined the percent deamidation of a particular peptide by dividing the mass area of the deamidated peptide by the sum of the area of the deamidated and native peptides. Considering the number of possible deamidation sites, isobaric species that are deamidated at different sites may co-migrate at a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent of the site of deamidation. This approach allows the definition of the specific sites involved in deamidation and the potential combinations involved in deamidation.

Secondary data processing: Secondary analysis of raw mass spectrometry was performed at the University of Maryland, Baltimore County using the following method. Peaks Studio v5.3 software (Bioinformatics Solutions Inc.) was used for all mass spectrometry analysis. Data refinement of the raw data files was performed with the following parameters: a precursor m/z tolerance of ≤10 ppm, and precursor charge state with a minimum of 2, maximum of 4. De novo sequencing of the input spectrum was performed using the Peaks algorithm with a precursor ion error tolerance of 10 ppm and product ion error tolerances of 0.1 Da. The digestion enzyme was set as trypsin, the variable modifications were oxidation, phosphorylation, and deamidation, and the fixed modification was carbamidomethylation of cysteine.

### 4. Structural Analysis of the AAV Capsid

We obtained the AAV8 atomic coordinates, structural factors, and associated capsid model from the RCSB Protein Data Bank (PDB ID: 3RA8). We performed structure refinement and generated an electron density independent of the primary amino acid sequence of AAV8 VP3 for use in three-dimensional (3D) structural analysis of the capsid. We performed this analysis in order to observe the isoaspartic acid electron density in the AAV8 capsid that was not biased by the expected primary sequence of AAV8 VP3. Using the resulting structure, we modeled the four asparagines in the AAV8 VP3 primary sequence with N+1 glycines as isoaspartic acids and then refined the AAV8 capsid structure using Crystallography and NMR System (CNS) software by strictly imposing the icosahedral non-crystallographic matrices using the standard refinement protocol (Brunger A T, et al. Acta Crystallogr D Biol Crystallogr 1998; 54 (Pt 5): 905-21). We obtained a structural model of isoaspartic acid from the HIC-UP database, followed by generation of a molecular dictionary in PRODRG for structure refinement (Kleywegt G J Acta Crystallogr D Biol Crystallogr 2007; 63 (Pt 1): 94-100). We then calculated the average electron density map of the AAV8 capsid (also in CNS) and visualized it using COOT software, followed by minor adjustments of the resulting model to fit the modeled isoaspartic acid residues into the electron density map (Emsley P and Cowtan K Acta Crystallogr D Biol Crystallogr 2004; 60 (Pt 12 Pt 1): 2126-32). We repeated this protocol to additionally model N512 in the AAV9 VP3 primary sequence with N+1 glycines (PDB ID: 3UX1). We generated all figures using COOT, PyMol, and UCSF Chimera (Emsley P and Cowtan K Acta Crystallogr D Biol Crystallogr 2004; 60 (Pt 12 Pt 1): 2126-32; DeLano W L PyMOL: An Open-Source Molecular Graphics Tool Vol. 40, 2002:82-92; Pettersen E F, et al. J Comput Chem 2004; 25 (13): 1605-12). We obtained a number of structures of previously identified deamidated proteins (PDB IDs: IDY5, 4E7G, IRTU, 1W9V, 4E7D, and 1C9D) for comparison of their electron density map for deamidated isoaspartic acid residues with our modeled isoaspartic acid residues from AAV8 and AAV9 (Rao F V, et al. Chem Biol 2005; 12 (1): 65-76; Noguchi S, et al. Biochemistry 1995; 34 (47): 15583-91; Esposito L, et al. J Mol Biol 2000; 297 (3): 713-32).

We determined temperature factors for deamidated residues by averaging the temperature factors for each atom of each asparagine residue reported in the AAV8 or AAV9 crystal structure atomic coordinates (PDB ID: 3RA8, 3UX1).

### 5. Animal Studies

The Institutional Animal Care and Use Committee of the University of Pennsylvania approved all animal procedures. To evaluate vector performance, we injected eight-week-old C57BL/6 mice intravenously via tail vein injection with 3e10 GCs of wtAAV8 or capsid mutant vector in a volume of 100 μL. All mice were sacrificed at day 14. For in vivo evaluation of luciferase expression, mice (˜20 g) were anesthetized and injected intraperitoneally with 200 μL or 15 mg/mL luciferin substrate (Perkin Elmer, Waltham, MA). Mice were imaged five minutes after luciferin administration and imaged via an IVIS Xenogen In Vivo Imaging System. We used Living Image 3.0 software to quantify signal in the described regions of interest. We took measurements at days 7 and 14.

### 6 Evaluation of Mutant Vector Titer and In Vitro Transduction Efficiency

We determined vector titers by qPCR of the DNAseI-resistant genomes. The qPCR primers anneal to the polyadenylation sequence of the packaged transgene. For in vitro evaluation of vector transduction efficiency by luciferase expression, we seeded 0.9e5 Huh7 cells/well in a black-walled 96-well plate in complete DMEM (10% fetal bovine serum, 1% penicillin/streptomycin). The next day, we removed the media and replaced it with 50 μL crude or purified vector diluted in complete media. We tested 4 dilutions in a 3 fold dilution series for each crude vector sample. After 48 hours, we prepared luciferin (Promega, Madison, WI) in complete media at 0.3 μg/μL and added it to transduced cells in a volume of 50 μL. Results were read on a Biotek Clarity luminometer. We find that luciferase activity/GC added to target cells is constant over a wide range of GCs, but can become saturated at high MOIs. Thus we inspect the dilution series data (luminescent units vs GC) for linearity, exclude the highest point if saturation is evident, and calculate an average Luciferase/GC for values in the linear range of each assay for each variant. This yields a transduction efficiency value. The data are normalized to simplify comparison by setting the wt control to a value of 1.

### 7. Biodistribution

We extracted DNA from liver samples using the QIAamp DNA Mini Kit (Qiagen, Hilden, Germany), and then analyzed the DNA for vector GC by real-time PCR as described previously with a primer/probe set designed against the RBG polyadenylation signal of the transgene cassette (Chen S J, et al. Hum Gene Ther Clin Dev 2013; 24 (4): 154-60).

Primer Sequences for AAV8 Mutants

| | Sequence | Description |
|---|---|---|
| SED ID NO: 56 | CGACAACCGGGCAAAACcagAATAGCAACTTTGCCTGG | QC mutagenic primers to |
| | | change AAV8 N499 to Q |
| SED ID NO: 57 | CCAGGCAAAGTTGCTATTCTGGTTTTGCCCGGTTGTCG | QC mutagenic primers to |
| | | change AAV8 N499 to Q |
| SED ID NO: 58 | GACAACCGGGCAAAACgacAATAGCAACTTTGCCTG | QC mutagenic primers to |
| | | change AAV8 N499 to D |
| SED ID NO: 59 | CAGGCAAAGTTGCTATTGTCGTTTTGCCCGGTTGTC | QC mutagenic primers to |
| | | change AAV8 N499 to D |
| SED ID NO: 60 | GGAGGCACGGCAcagACGCAGACTCTGGG | qc mutagenic primers to |
| | | change AAV8 N459 to Q |
| SED ID NO: 61 | CCCAGAGTCTGCGTCTGTGCCGTGCCTCC | qc mutagenic primers to |
| | | change AAV8 N459 to Q |
| SED ID NO: 62 | CAGGAGGCACGGCAgatACGCAGACTCTGG | qc mutagenic primers to |
| | | change AAV8 N459 to D |
| SED ID NO: 63 | CCAGAGTCTGCGTATCTGCCGTGCCTCCTG | qc mutagenic primers to |
| | | change AAV8 N459 to D |
| SED ID NO: 64 | ctcctcccgatgtcgcgttggagatttgc | AAV8 NA263 F |
| SED ID NO: 65 | gcaaatctccaacgcgacatcgggaggag | AAV8 NA263 R |
| SED ID NO: 66 | cccacggcctgactagcgttgttgagtgtta | AAV8 NA385 F |
| SED ID NO: 67 | taacactcaacaacgctagtcaggccgtggg | AAV8 NA385 R |
| SED ID NO: 68 | ggattagccaatgaatttcttgcattcagatggtatttggtcc | AAV8 NA514 F |
| SED ID NO: 69 | ggaccaaataccatctgaatgcaagaaattcattggctaatcc | AAV8 NA514 R |
| SED ID NO: 70 | tttgccaaaaatcaggatcgcgttactgggaaaaaaacg | AAV8 NA540 F |
| SED ID NO: 71 | cgtttttttcccagtaacgcgatcctgatttttggcaaa | AAV8 NA540 R |
| SED ID NO: 72 | ggacccttcaacgcactcgacaagggg | AAV8 NA57 F |
| SED ID NO: 73 | ccccttgtcgagtgcgttgaagggtcc | AAV8 NA57 R |
| SED ID NO: 74 | tggctcctcccgatgtgctgttggagatttgcttg | AAV8 N5263 F |
| SED ID NO: 75 | caagcaaatctccaacagcacatcgggaggagcca | AAV8 NS263 R |
| SED ID NO: 76 | cccacggcctgactactgttgttgagtgttagg | AAV8 NS385 F |
| SED ID NO: 77 | cctaacactcaacaacagtagtcaggccgtggg | AAV8 NS385 R |
| SED ID NO: 78 | ttagccaatgaatttctgctattcagatggtatttggtcccagcag | AAV8 NS514 F |
| SED ID NO: 79 | ctgctgggaccaaataccatctgaatagcagaaattcattggctaa | AAV8 NS514 R |
| SED ID NO: 80 | ttgtttgccaaaaatcaggatgctgttactgggaaaaaaacgctc | AAV8 NS540 F |
| SED ID NO: 81 | gagcgtttttttcccagtaacagcatcctgatttttggcaaacaa | AAV8 NS540 R |
| SED ID NO: 82 | ctcccccttgtcgaggctgttgaagggtccgag | AAV8 NS57 F |
| SED ID NO: 83 | ctcggacccttcaacagcctcgacaagggggag | AAV8 NS57 R |
| SED ID NO: 84 | cagcgactcatcaacGACaactggggattccg | QC primer for AAV8 N305D |
| SED ID NO: 85 | ggaggcacggcaGATacgcagactctgg | QC primer for AAV8 N459D |
| SED ID NO: 86 | gacaaccgggcaaaacGACaatagcaactttgcctg | QC primer for AAV8 N499D |
| SED ID NO: 87 | ccatctgaatggaagaGATtcattggctaatcctggcatc | QC primer for AAV8 N517D |
| SED ID NO: 88 | cgaagcccaaagccGACcagcaaaagcagg | QC primer for AAV8 N35D |
| SED ID NO: 89 | gtacctgcggtatGACcacgccgacgcc | QC primer for AAV8 N94D |
| SED ID NO: 90 | gatgctgagaaccggcGACaacttccagtttacttac | QC primer for AAV8 N410D |
| SED ID NO: 91 | cagactctgggcttcagcGATggtgggcctaatacaatg | QC primer for AAV8 Q467D |
| SED ID NO: 92 | ccaatcaggcaaagGACtggctgccaggac | QC primer for AAV8 N479D |
| SED ID NO: 93 | cacggacggcGACttccacccgtctc | QC primer for AAV8 N630D |
| SED ID NO: 94 | gatcctgatcaagGACacgcctgtacctgcg | QC primer for AAV8 N653D |
| SED ID NO: 95 | gtacctcggacccttcCAGggactcgacaaggg | QC primer for AAV8 N57Q |
| SED ID NO: 96 | ctacaagcaaatctccCAGgggacatcgggaggagc | QC primer for AAV8 N263Q |
| SED ID NO: 97 | gctacctaacactcaacCAGggtagtcaggccgtgg | QC primer for AAV8 N385Q |
| SED ID NO: 98 | gctgggaccaaataccatctgCAGggaagaaattcattggc | QC primer for AAV8 N514Q |
| SED ID NO: 99 | ggagcgtttttttcccagtCAGgggatcctgatttttggc | QC primer for AAV8 N540Q |
| SED ID NO: 100 | cggaatccccagttgtcgttgatgagtcgctg | QC primer for AAV8 N305D |
| SED ID NO: 101 | ccagagtctgcgtatctgccgtgcctcc | QC primer for AAV8 N459D |
| SED ID NO: 102 | caggcaaagttgctattgtcgttttgcccggttgtc | QC primer for AAV8 N499D |
| SED ID NO: 103 | gatgccaggattagccaatgaatctcttccattcagatgg | QC primer for AAV8 N517D |
| SED ID NO: 104 | cctgcttttgctggtcggctttgggcttcg | QC primer for AAV8 N35D |
| SED ID NO: 105 | ggcgtcggcgtggtcataccgcaggtac | QC primer for AAV8 N94D |
| SED ID NO: 106 | gtaagtaaactggaagttgtcgccggttctcagcatc | QC primer for AAV8 N410D |
| SED ID NO: 107 | cattgtattaggcccaccatcgctgaagcccagagtctg | QC primer for AAV8 Q467D |
| SED ID NO: 108 | gtcctggcagccagtcctttgcctgattgg | QC primer for AAV8 N479D |
| SED ID NO: 109 | gagacgggtggaagtcgccgtccgtg | QC primer for AAV8 N630D |
| SED ID NO: 110 | cgcaggtacaggcgtgtccttgatcaggatc | QC primer for AAV8 N653D |
| SED ID NO: 111 | gcagcgactcatcaacGACaactggggattccggc | alternative longer primer |
| | | to make AAV8 N305D by |
| | | qc mutagenesis |
| SED ID NO: 112 | GCCGGAATCCCCAGTTGTCGTTGATGAGTCGCTGC | alternative longer primer |
| | | to make AAV8 N305D by |
| | | qc mutagenesis |
| SED ID NO: 113 | cagcgactcatcaacGACaactggggattccggc | alternative longer primer |
| | | to make AAV8 N305D by |
| | | qc mutagenesis |
| SED ID NO: 114 | GCCGGAATCCCCAGTTGTCGTTGATGAGTCGCTG | alternative longer primer |
| | | to make AAV8 N305D by |
| | | qc mutagenesis |
| SED ID NO: 115 | gcgactcatcaacGACaactggggattccg | alternative shorter primer |
| | | to make AAV8 N305D by |
| | | qc mutagenesis |
| SED ID NO: 116 | CGGAATCCCCAGTTGTCGTTGATGAGTCGC | alternative shorter primer |
| | | to make AAV8 N305D by |
| | | qc mutagenesis |
| SED ID NO: 117 | ctctgggcttcagcGAAggtgggcctaatac | mutagenic QC primer to |
| | | make AAV8 Q467E |
| SED ID NO: 118 | GTATTAGGCCCACCTTCGCTGAAGCCCAGAG | mutagenic QC primer to |
| | | make AAV8 Q467E |
| SED ID NO: 119 | cctcggacccttcGACggactcgacaagg | QC primer for AAV8 N57D |
| SED ID NO: 120 | tacaagcaaatctccGACgggacatcgggaggag | QC primer for AAV8 N263D |
| SED ID NO: 121 | ctacctaacactcaacGACggtagtcaggccgtg | QC primer for AAV8 N385D |
| SED ID NO: 122 | ctgggaccaaataccatctgGATggaagaaattcattggctaatc | QC primer for AAV8 N514D |
| SED ID NO: 123 | gagcgtttttttcccagtGACgggatcctgatttttggc | QC primer for AAV8 N540D |
| SED ID NO: 124 | ccttgtcgagtccgtcgaagggtccgagg | QC primer for AAV8 N57D |
| SED ID NO: 125 | ctcctcccgatgtcccgtcggagatttgcttgta | QC primer for AAV8 N263D |
| SED ID NO: 126 | cacggcctgactaccgtcgttgagtgttaggtag | QC primer for AAV8 N385D |
| SED ID NO: 127 | gattagccaatgaatttcttccatccagatggtatttggtcccag | QC primer for AAV8 N514D |
| SED ID NO: 128 | gccaaaaatcaggatcccgtcactgggaaaaaaacgctc | QC primer for AAV8 N540D |

### B. Results

### AAV8 Shows Substantial Charge Heterogeneity in its Capsid Proteins

To qualitatively assess the presence of post-translational modifications on the AAV8 vector capsid that could affect vector performance, we analyzed AAV8 total capsid protein purified by iodixanol gradient both by 1D and 2D gel electrophoresis. In a 1D reducing sodium dodecyl sulfate SDS gel, VP1, VP2, and VP3 resolved as single bands at the appropriate molecular weights (FIG. 1B) (Rose J A, et al. J Virol 1971; 8 (5): 766-70). When further evaluated by 2D gel electrophoresis, which separates proteins based on charge (FIG. 1C), each of the capsid proteins additionally resolved as a series of distinct spots with different isoelectric points (pIs) ranging from pH 6.3 to >7.0 dependent on the VP isoform (FIG. 1D). Individual spots on each VP were separated by discrete intervals of 0.1 pI units as measured as migration relative to the carbonic anhydrase isoform internal isoelectric point standards, suggesting a single residue charge change. The presence of these isoforms suggests that each VP has the potential to undergo many modifications, thereby causing them to migrate differently under isoelectric focusing.

Deamidation, in which a fraction of (typically asparagine) side-chain amide groups are converted to carboxylic acid (FIG. 1A), is a common source of charge heterogeneity in protein preparations. To determine if deamidation could be responsible for the distinct population of VP charge isoforms, we mutated two AAV8 asparagine residues individually to aspartate. These capsid mutations should shift the charge by an amount equivalent to the complete deamidation of a single additional asparagine residue. 2D gel analysis of the mutants indicates the major spots for VP1, VP2, and VP3 shifted one spot location more acidic (0.1 pH units) than the equivalent spots in wild-type (wt) AAV8 (FIG. 1E-FIG. 1G). The magnitude of this shift is equivalent to the observed spacing between the wt VP charge isoforms. Thus, the 2D gel patterning of AAV capsid proteins is consistent with multi-site deamidation.

### Spontaneous Deamidation Occurs on the AAV8 Vector Capsid

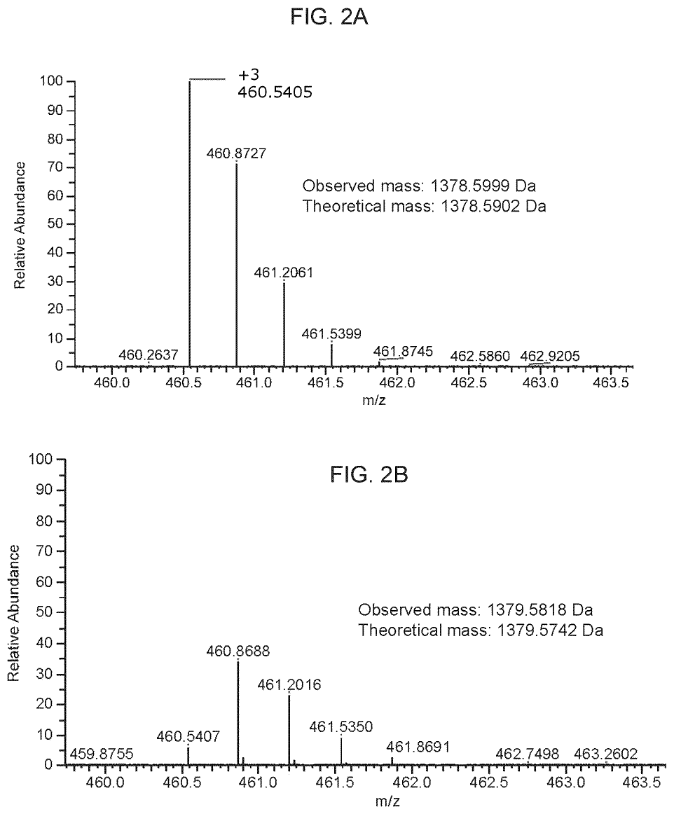

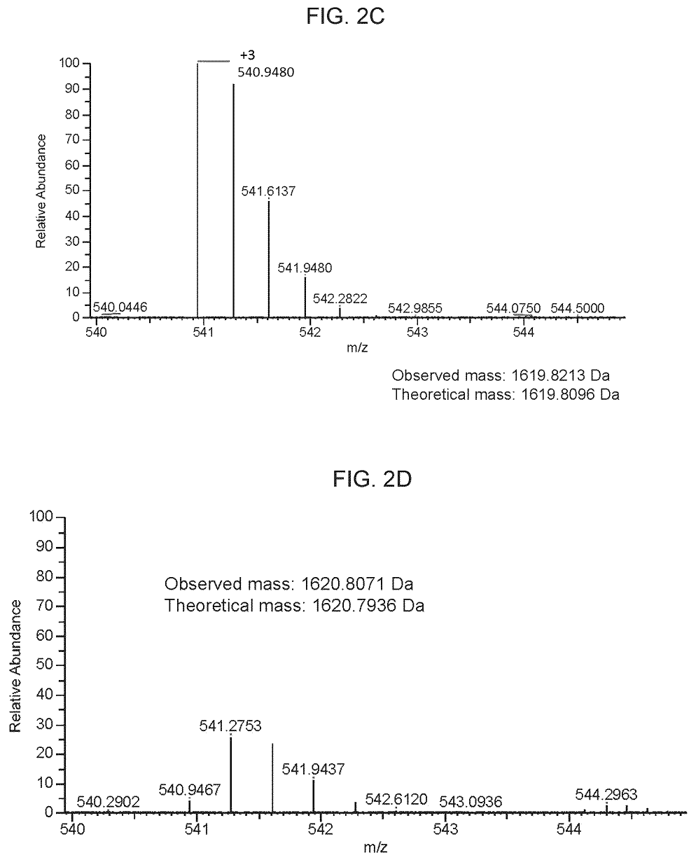

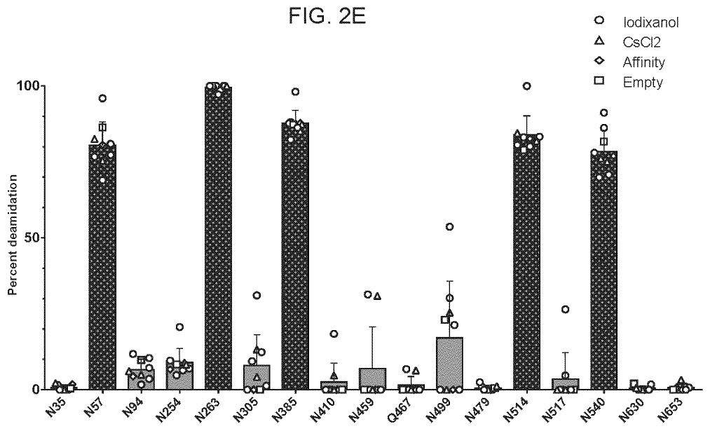

To identify modifications responsible for the discrete spotting pattern for each capsid protein, we analyzed a panel of AAV8 vectors by mass spectrometry. Coverage of the AAV8 capsid protein averaged >95% of the total VP1 sequence (data not shown). We detected extensive deamidation of a subset of asparagine and glutamine residues by mass spectroscopy, which showed an increase of ˜1 Da in the observed mass of the individual peptides as compared to predicted values based on the sequence encoded by the DNA; we observed this pattern of deamidation in all preparations of AAV8 vectors (FIG. 2A-FIG. 2D).

To evaluate the global heterogeneity of deamidation between commonly used purification methods and to examine deamidation in the VP1 and VP2 unique regions, we selected nine lots of AAV8 produced by triple transfection in 293 cells and purified them by either cesium chloride gradient, iodixanol gradient, or affinity chromatography. Vectors also varied with respect to promoters and transgene cassettes. To determine if the presence of the vector genome had an impact on deamidation, we also evaluated an AAV8 prep produced by triple transfection in 293 cells in the absence of cis plasmid (producing empty capsids only) and purified by iodixanol gradient.

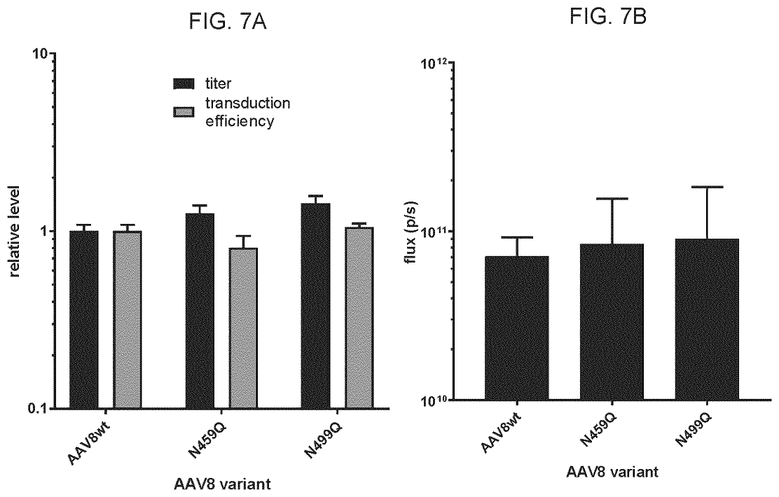

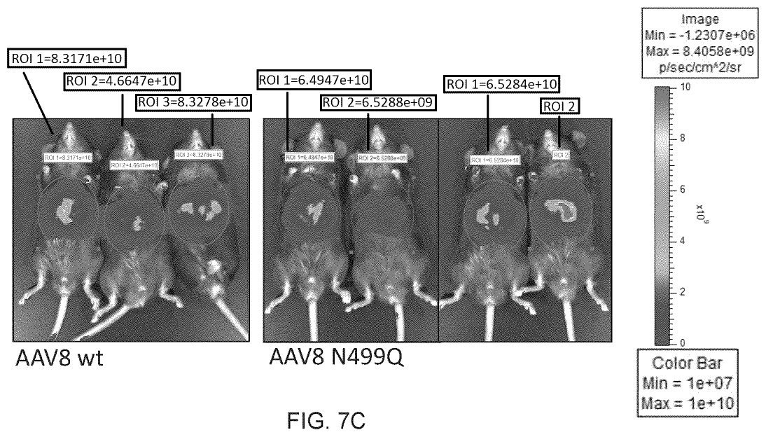

A wide range of deamidation was present across asparagine and glutamine residues of the AAV8 capsid, ranging from undetectable to over 99% of individual amino acids being deamidated (FIG. 2E). The highest levels of deamidation (>75%) occurred at asparagine residues where the N+1 residue was glycine (i.e., NG pairs) (Table 1). We detected lower levels of deamidation (i.e., up to 17%) at additional asparagine residues where the N+1 was not glycine. The average deamidation for asparagines was largely consistent between preps. We also detected deamidation at glutamine residues but at a lower frequency than at asparagines; the highest percent we observed was <2% at Q467 (FIG. 7). This observation was inconsistent across preparations (data not shown). We observed the greatest preparation-to-preparation differences at residue N499 (N+1 residue is asparagine), with values ranging from <1% to over 50% deamidation. Regardless, the variations we observed in deamidation between preparations of vector did not appear to be related to purification method, transgene identity, or the presence of vector genome, suggesting that these factors do not impact deamidation rates.

TABLE 1
Characteristics of AAV8 deamidated residues of interest.

| | | | | | Temper- |
|---|---|---|---|---|---|
| | | | | | ature |
| | N + 1 | Structural | Structural | Average % | factor |
| | residue | topology | motif | deamidation | (Å\hat{ }2) |
| N35 | Q | N/A | N/A | 1 | N/A |
| N57 | G | N/A | N/A | 80 | N/A |
| N94 | H | N/A | N/A | 7 | N/A |
| N254* | N | Surface | Not | 9 | 35 |
| | | exposed | assigned | | |
| N255* | H | Surface | Not | N/A | 42 |
| | | exposed | assigned | | |
| N263 | G | Surface | HVR I | 99 | 51 |
| | | exposed | | | |
| N305 | N | Buried | Alpha helix | 8 | 33 |
| N385 | G | Surface | HVR III | 88 | 41 |
| | | exposed | | | |
| N410 | N | Buried | Not | 3 | 33 |
| | | | assigned | | |
| N459 | T | Surface | HVR IV | 7 | 65 |
| | | exposed | | | |
| N499 | N | Surface | HVR V | 17 | 45 |
| | | exposed | | | |
| N514* | G | Surface | HVR V | 84 | 36 |
| | | exposed | | | |
| N517* | S | Surface | HVR V | 4 | 40 |
| | | exposed | | | |
| N540* | G | Buried | HVR VII | 79 | 40 |
| N630* | F | Buried | Not | 1 | 32 |
| | | | assigned | | |
| N653 | T | Surface | HI loop | 1 | 35 |
| | | exposed | | | |

Asterisks represent residues selected for further analysis.

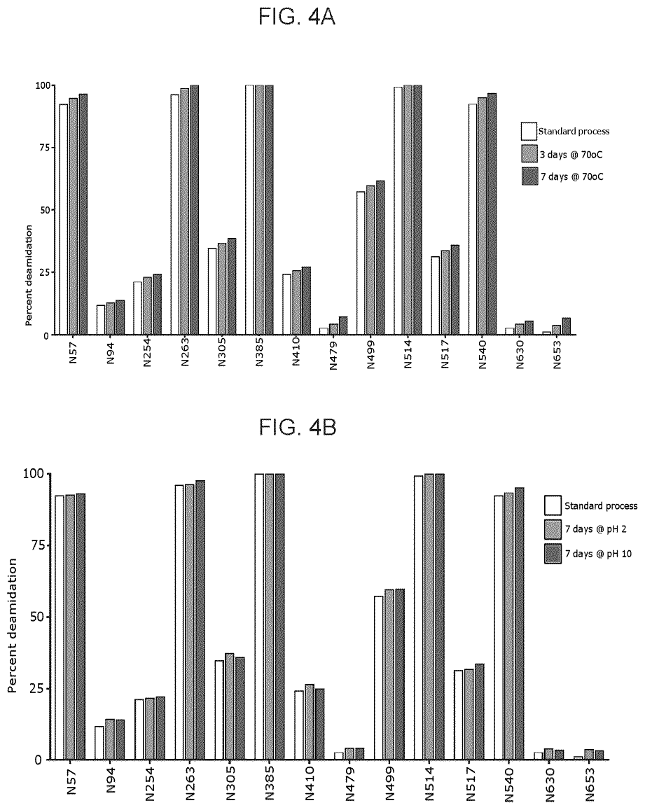

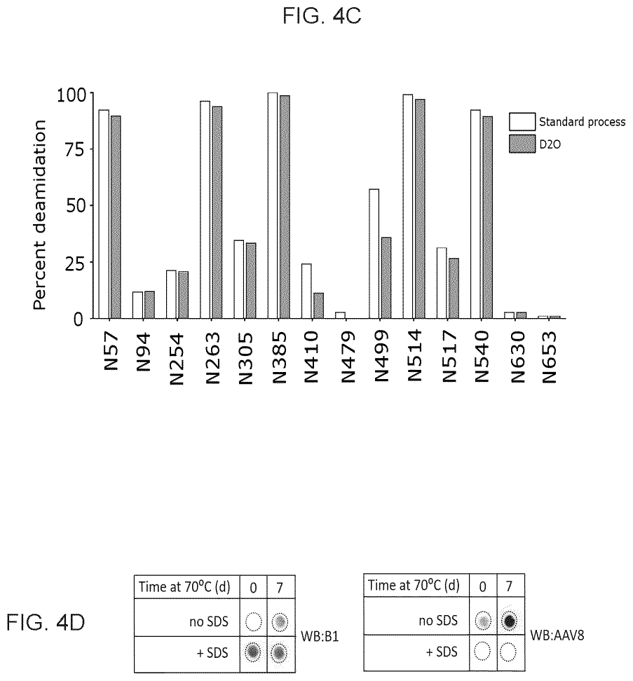

Next, we ran a series of experiments to determine if sample handling contributed to the observed levels of deamidation in AAV8. Extreme temperature (70° C. for 7 days) or pH (pH 2 or pH 10 for 7 days) did not significantly induce additional deamidation in the AAV8 capsid (FIG. 4A and FIG. 4B). Given this resistance, we reason that it was unlikely that the deamidation observed occurred only in the purification phase, which was shorter and relatively mild in comparison. We attempted to perform mass spectrometry analysis on unpurified vector to determine the extent of deamidation before and after purification, but were unsuccessful. Likewise, heavy water controls indicate that processing specific to our mass spectrometry workflow do not contribute additional deamidation events (FIG. 4C).

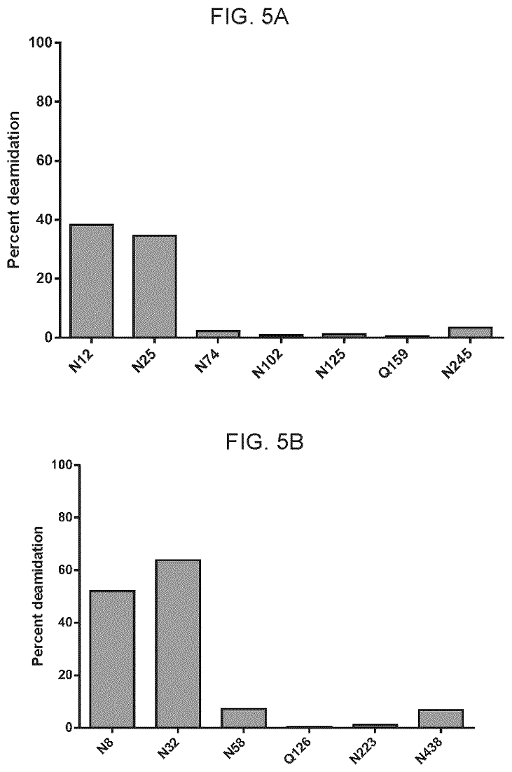

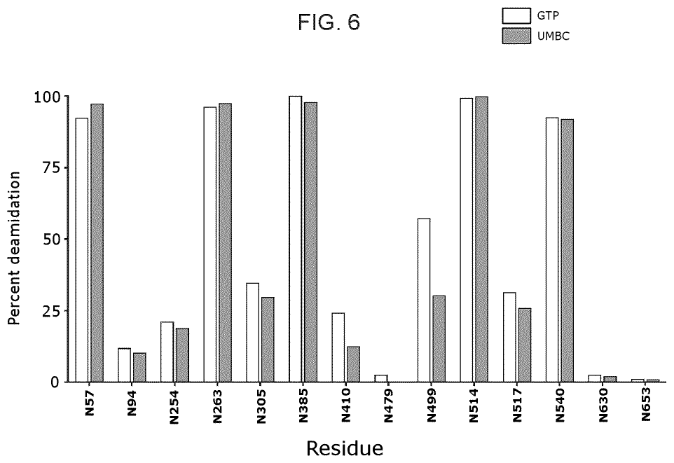

To validate our mass spectrometry workflow, we examined two recombinant proteins that have been evaluated previously for deamidation; our findings (FIG. 5A and FIG. 5B) agree with the published results [Henderson, L E, Henriksson, D, and Nyman, P O (1976). Primary structure of human carbonic anhydrase C. The Journal of biological chemistry 251:5457-5463 and Carvalho, R N, Solstad, T, Bjorgo, E, Barroso, J F, and Flatmark, T (2003). Deamidations in recombinant human phenylalanine hydroxylase. Identification of labile asparagine residues and functional characterization of Asn-->Asp mutant forms. The Journal of biological chemistry 278:15142-1515]. Additionally, we engaged a secondary institution to evaluate our raw data from AAV8. This independent analysis identified the same sites as deamidated, with minimal variation in the extent of modification at each site attributable to software-to-software variations in peak detection and area calculation (FIG. 6).

### Structural Topology, Temperature Factor, and the Identity of the N+1 Amino Acid Contribute to Deamidation Frequency

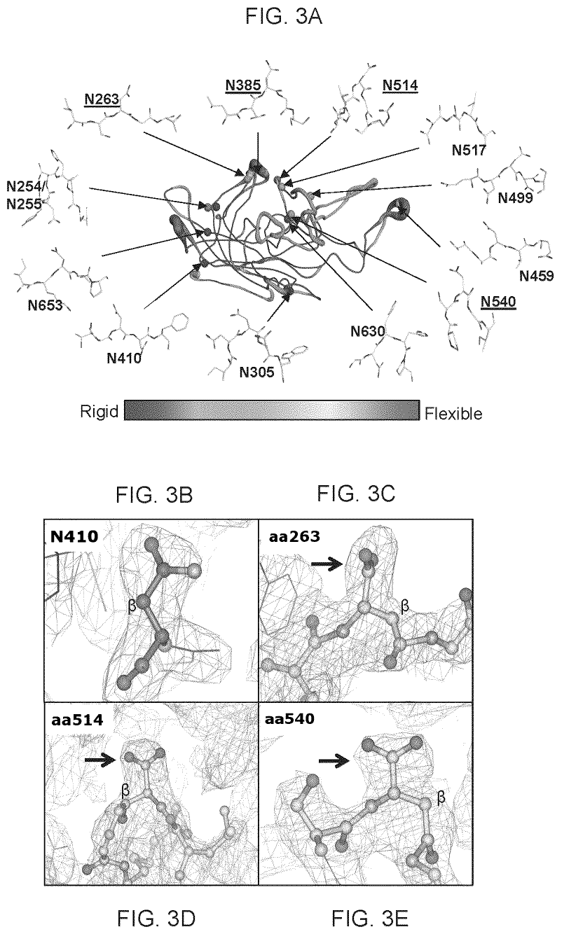

As the structure of AAV8 has been solved and published (PDB identifier: 2QA0) (Nam H J, et al. J Virol 2011; 85 (22): 11791-99), we next examined the AAV8 capsid structure for evidence of favorable conditions for non-enzymatic deamidation and to correlate percent deamidation with established structural features (Nam H J, et al. J Virol 2007; 81 (22): 12260-71). We focused on asparagine residues exclusively, as the factors influencing asparagine deamidation are better characterized in the literature and asparagine deamidation events are far more common than glutamine deamidation events (Robinson, N E, and Robinson, A B (2001). Molecular clocks. Proc Natl Acad Sci USA 98:944-949). We also determined the temperature (or B) factor for each of these residues from the AAV8 crystal structure; temperature factor is a measure of the displacement of an atom from its mean position, with higher values indicating a larger displacement, higher thermal vibration, and therefore increased flexibility (Parthasarathy S and Murphy M R. Protein Science: A Publication of the Protein Society 1997; 6:2561-7). The majority of asparagines of interest were located in or near the surface-exposed HVRs (Table 1), which are structurally favorable for deamidation and provide a solvent-exposed environment (Govindasamy L, et al. J Virol 2013; 87 (20): 11187-99). We found that residues located in these flexible loop regions were, on average, more frequently deamidated than residues in less flexible regions such as beta strands and alpha helices. For example, the NG residue at position N263 is part of HVR I, has a high temperature factor, and was >98% deamidated on average (FIG. 7A and FIG. 6, Table 1). N514, which was deamidated ˜85% of the time (FIG. 3 and FIG. 6, Table 1), is also in an HVR (HVR V) with an N+1 glycine; however, the local temperature factor is relatively low in comparison to that of N263 due to its interaction with residues on other VP monomers at the three-fold axis. Less-favorable +1 residues and lower local temperature factors correlated with lower deamidation, even for HVR residues. For example, N517 was on average only 4% deamidated (Table 1); this residue has an equivalent temperature factor to the highly deamidated N514, but its N+1 residue is a serine, decreasing the likelihood of deamidation events due to steric hindrance. This demonstrates that a number of factors cumulatively determine the extent of deamidation at a given capsid position, although the identity of the +1 residue is apparently the most influential factor.

To test the role of the +1 residue in asparagine deamidation, we generated mutant vectors in which AAV8 NG sites were individually mutated at the +1 position to either alanine or serine. Model peptide studies indicate that NG peptides deamidate with a half-life as short as 1 day, whereas NA or NS peptides typically deamidate 25- or 16-fold more slowly, respectively (Robinson N E and Robinson A B. Proc Natl Acad Sci USA. 2001; 98 (8): 4367-72). Mass spectrometry analysis of the vector mutants confirmed the central role of the +1 site in determining the extent of vector deamidation. NG sites in this set (>80% deamidation in wt) showed selective stabilization of the adjacent asparagine when the +1 site was changed to alanine (<5% deamidation) or serine (<14% deamidation) (Table 2).

TABLE 2
Extent of deamidation (%) at
five AAV8 NG sites in wt and six +1 site mutants

| posi- | | | | | | | |
|---|---|---|---|---|---|---|---|
| tion\ | WT | | | | | | |
| variant | (average) | G58S | G58A | G264A | G386S | G386A | G515A |
| N57 | 81.8 | 8.4 | 1.9 | 89.7 | 89.7 | 91.6 | 93.6 |
| N263 | 99.3 | 98.2 | 98.9 | 4.8 | 100.0 | 94.5 | 97.2 |
| N385 | 89.1 | 96.3 | 94.8 | 97.1 | 13.5 | 2.5 | 97.0 |
| N514 | 85.2 | 100.0 | 98.0 | 98.8 | 100.0 | 100.0 | 2.2 |
| N540 | 84.5 | 95.0 | 92.6 | 97.9 | 96.9 | 86.1 | 89.5 |

Residues that were at least partially buried and not readily exposed to solvent and/or were located in regions of low local flexibility in the intact, fully assembled AAV8 capsid had a lower frequency of deamidation compared to those located in a more favorable environment Table 1). Despite this, a few of the residues in unfavorable conditions were deamidated. For example, N630 is at least partially buried but still had a detectable degree of deamidation. For this residue, the presence of phenylalanine as the N+1 residue suggests that this region could be a novel site of non-enzymatic autoproteolytic cleavage within the AAV8 VP3 protein.

### Structural Modeling of AAV8 VP3 Confirms Deamidation Events

To provide direct evidence of deamidation in the context of an assembled capsid, we evaluated the crystal structure of AAV8 (Nam H-J, et al. J Virol 2011; 85 (22): 11791-9). The resolution of the available crystal structure (i.e., 2.7 Å) of this serotype is not high enough to identify the terminal atoms in the R groups and, therefore, is insufficient to directly distinguish between asparagine, aspartic and isoaspartic acid residues. Other aspects of the structure of the isomer of aspartic acid that forms under these conditions provided us an opportunity to determine deamidation from the 2.7 Å structure. This analysis was based on two assumptions: 1) The predominant product of spontaneous deamidation of an asparagine is isoaspartic rather than aspartic acid, which is generated at a 3:1 ratio (Geiger T and Clarke S. J Biol Chem 1987; 262 (2): 785-94), and 2) an asparagine or aspartic acid can be differentiated from an isoaspartic acid because the electron density map corresponding to the R group of isoaspartic acid is shorter in length. This shorter R group is created when the beta carbon from the R group of isoaspartic acid is lost when incorporated into the main chain of the AAV8 VP3 capsid protein backbone following resolution of the succinimidyl intermediate during the deamidation reaction.

We first refined the AAV8 structure itself, generating an AAV8 capsid electron density that was not biased by the known AAV8 VP3 sequence. We then examined the refined AAV8 crystal structure for evidence of deamidation based on the presence of a shorter R group associated with isoaspartic acid (FIG. 3A-FIG. 3E). The electron density map confirmed a shorter R group for the highly deamidated N+1 glycine residues at positions 263 (FIG. 3C), 385 (not shown), 514 (FIG. 3D), and 540 (FIG. 3E) when compared to the asparagine at 410 that had no deamidation detected by mass spectrometry (FIG. 3B). The deamidation indicated by the electron density map is therefore consistent with the data generated by mass spectrometry at these sites with >75% deamidation. The resulting isoaspartic acid models were comparable to isoaspartic acid residues observed in the crystal structures of other known deamidated proteins, supporting the validity of our analysis of AAV8 (Rao F V, et al. Chem Biol. 2005; 12 (1): 65-76; Noguchi S, et al. Biochemistry 1995; 34 (47): 15583-91; Esposito L, et al. J Mol Biol 2000; 297 (3): 713-32). This structural analysis serves as an independent confirmation of the deamidation phenomena observed when analyzing the AAV8 capsid via mass spectrometry.

### Deamidation of the AAV Capsid is not Serotype Specific

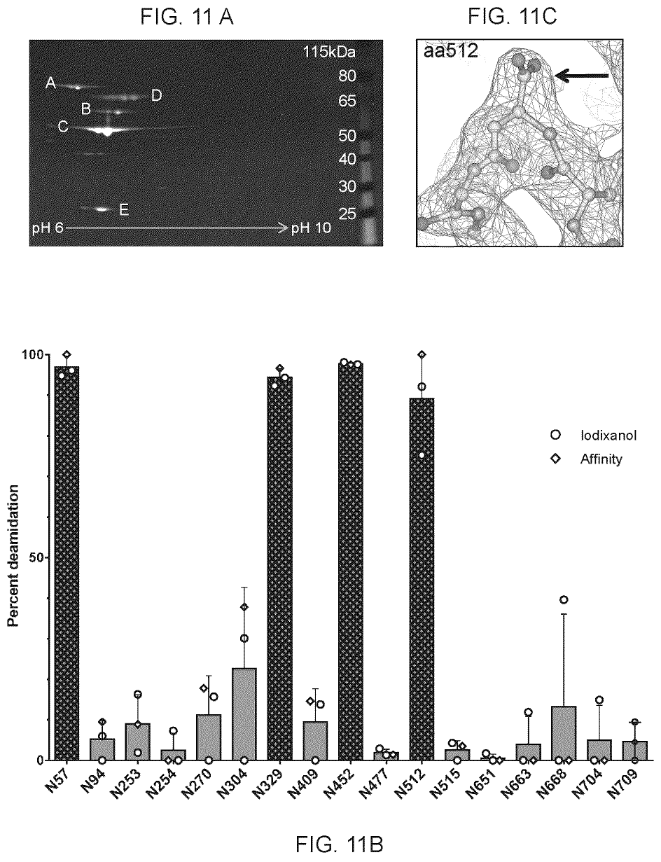

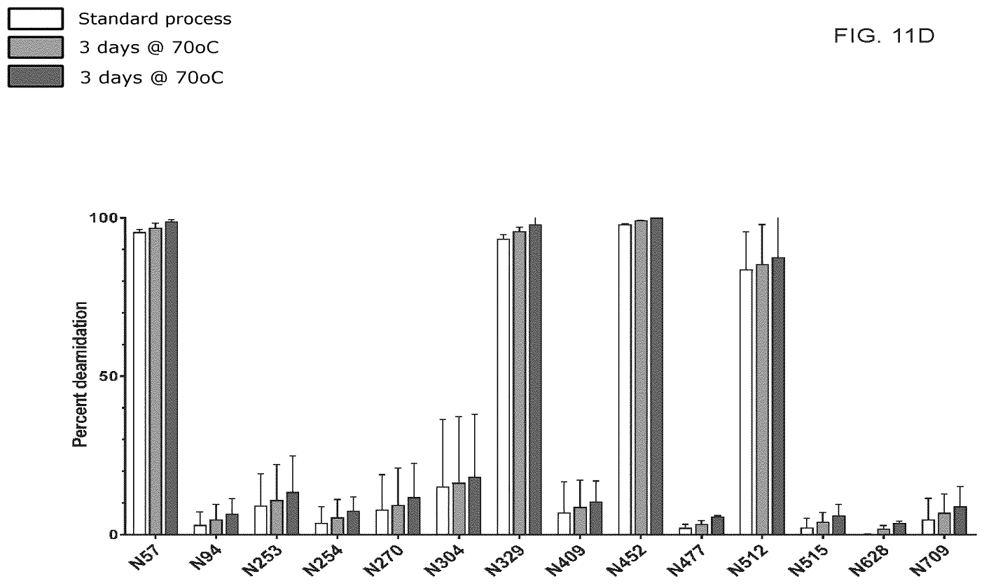

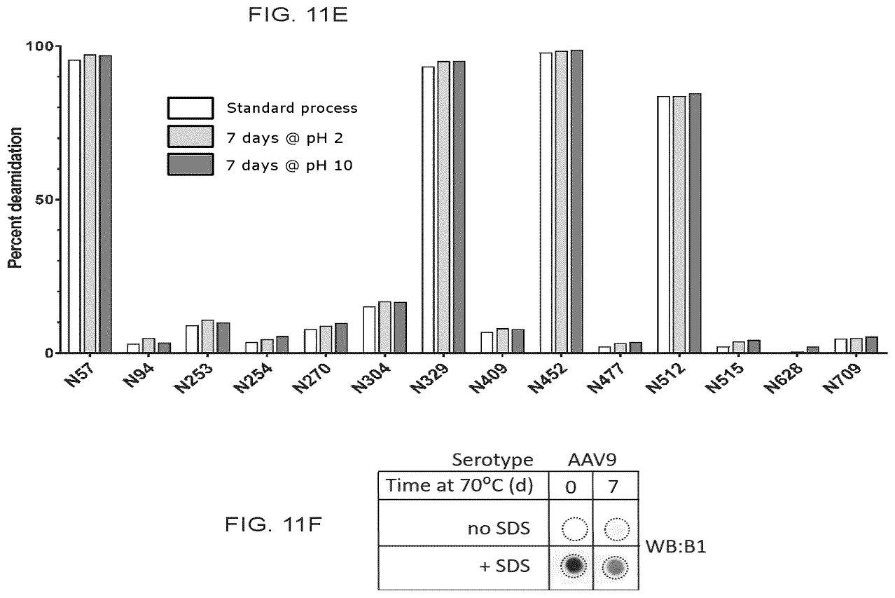

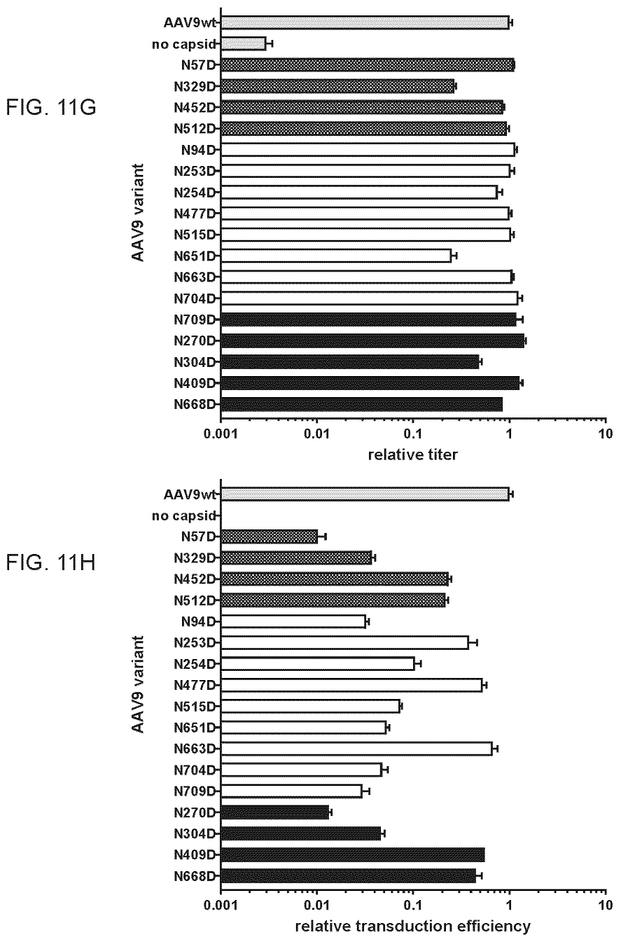

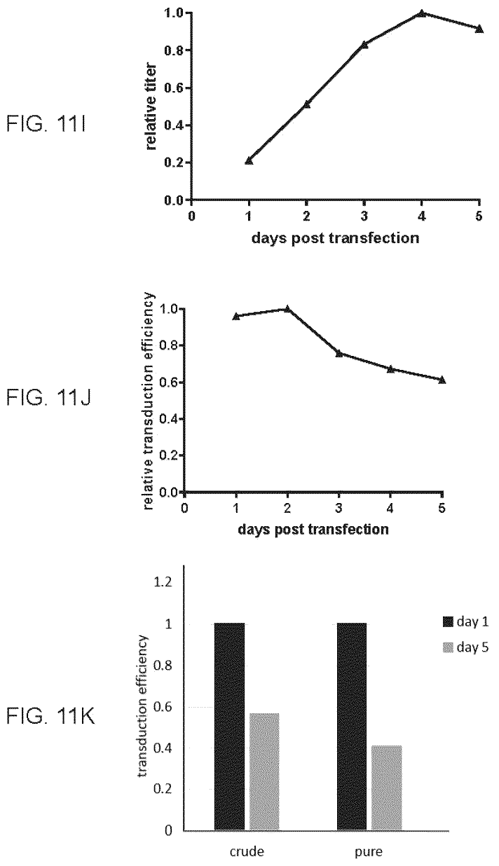

We investigated serotypes beyond AAV8 for evidence of capsid deamidation. We examined AAV9 vector preparations using 2D gel electrophoresis (FIG. 11A) and mass spectrometry (FIG. 11B), including controls for potential vector-processing effects (FIG. 11D-FIG. 11F). The pattern and extent of AAV9 deamidation was similar to that of AAV8. All four AAV9 NG sites were >85% deamidated; 13 non-NG sites were deamidated to lesser extent, with a few sites showing high lot-to-lot variability in % deamidation. Next, we applied our structural analysis workflow and refit existing AAV9 crystallographic data (FIG. 11C, Table 3). As with AAV8, isoaspartic acid fit better into the electron density of several NG sites in the AAV9 crystal structure. We extended our 2D gel analysis (data not shown) and mass spectrometry (summarized in Table 4) to five additional evolutionarily diverse serotypes (rh32.33, AAV7, AAV5, AAV4, AAV3B and AAV1). All of the capsids examined contain a similar pattern and extent of deamidation, indicating that this modification is widespread in clinically relevant AAV vectors, and is determined by similar underlying primary-sequence and structural factors.

TABLE 3
Characteristics of AAV9 deamidated residues of interest.

| | | | | | Temper- |
|---|---|---|---|---|---|
| | | | | | ature |
| | N + 1 | Structural | Structural | Average % | factor |
| | residue | topology | motif | deamidation | (Å\hat{ }2) |
| N57 | G | N/A | N/A | 97 | N/A |
| N94 | H | N/A | N/A | 5 | N/A |
| N253 | N | Surface | Not | 9 | 41 |
| | | exposed | assigned | | |
| N254 | H | Surface | Not | 2 | 50 |
| | | exposed | assigned | | |
| N270 | D | Surface | HVR I | 11 | 65 |
| | | exposed | | | |
| N304 | N | Buried | Alpha helix | 23 | 35 |
| N329 | G | Surface | HVR II | 94 | 89 |
| | | exposed | | | |
| N409 | N | Buried | Not | 9 | 36 |
| | | | assigned | | |
| N452 | G | Surface | HVR IV | 98 | 64 |
| | | exposed | | | |
| N477 | Y | Buried | Not | 2 | 33 |
| | | | assigned | | |
| N512 | G | Surface | HVR V | 89 | 48 |
| | | exposed | | | |
| N515 | S | Surface | HVR V | 3 | 47 |
| | | exposed | | | |
| N651 | T | Buried | HI loop | 1 | 38 |
| N663 | K | Surface | HI loop | 4 | 49 |
| | | exposed | | | |
| N668 | S | Surface | HI loop | 13 | 52 |
| | | exposed | | | |
| N704 | Y | Surface | HVR IX | 5 | 68 |
| | | exposed | | | |
| N709 | N | Surface | HVR IX | 5 | 55 |
| | | exposed | | | |

Conserved asparagine residues with homologous N + 1 residues (in comparison to AAV8) are denoted in italics (determined by alignment of the full-length amino acid sequences of AAV8 and AAV9 VP1).

TABLE 4
Extent of deamidation observed for diverse serotypes

| | | Average % | | | | |
|---|---|---|---|---|---|---|
| | vector | sequence | | | # of non NG | average non- |
| | preps | Coverage by | | average NG % | sites observed | NG % |
| serotype | analyzed | MS | # of NGs | deamidation | deamidated | deamidation |
| AAV1 | 3 | 91.4 | 4 | 95.6 | 19 | 12.9 |
| AAV3B | 1 | 89.8 | 4 | 97.0 | 9 | 9.4 |
| AAV4 | 3 | 84.7 | 4 | 96.2 | 15 | 15.3 |
| AAV5 | 1 | 88.7 | 3 | 88.7 | 11 | 15.3 |
| AAV7 | 1 | 90.9 | 4 | 92.1 | 9 | 13 |
| AAV8 | 21 | 93.4 | 5 | 90.5 | 37 | 7.4 |
| AAV9 | 7 | 90.2 | 4 | 95.5 | 26 | 5.3 |
| rh32.33 | 1 | 100 | 3 | 97.4 | 14 | 16.2 |

### Deamidation Events can Affect Capsid Assembly and Transduction Efficiency

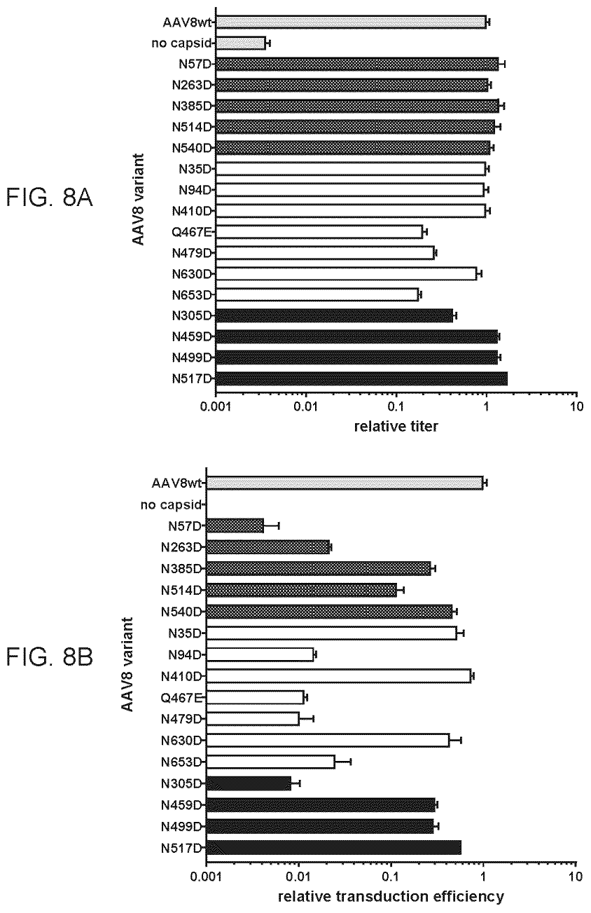

One approach to testing the functional impact of deamidation is by substituting asparagine with aspartate by genetic mutation. We generated an aspartate mutant vector encoding a luciferase reporter for each deamidated AAV8 asparagine by small-scale triple transfection of 293 cells, and titered the vectors by qPCR of DNAseI resistant genome copies (FIG. 8A). The mutations rarely affected capsid assembly relative to wtAAV8, and effects were limited to mostly buried, non-NG sites with low overall deamidation in the wt vector. Next, we assessed the mutation panel for in vitro transduction efficiency of human liver-derived Huh7 cells (FIG. 8B). Several mutants showed impaired transduction efficiency, with positions N57, N94, N263, N305, Q467, N479, and N653 exhibiting >10-fold transduction loss. We observed a similar number of sensitive sites for AAV9 (FIG. 11G and FIG. 11H). As typically only a fraction of residues at a given position are deamidated endogenously, this approach has the potential to overestimate functional loss for proteins such as capsids where the functional unit is a homomeric assembly; endogenous modification at one capsid site may be compensated for by a neighboring subunit with an intact residue. Nonetheless, we reasoned that the method could help prioritize deamidated residues for future monitoring during manufacturing or mutational stabilization. Functional data from populations of endogenously deamidating vectors will be required to place this loss-of-function mutagenesis data in the proper context.

Vector Activity Loss Through Time is Correlated with Progressive Deamidation

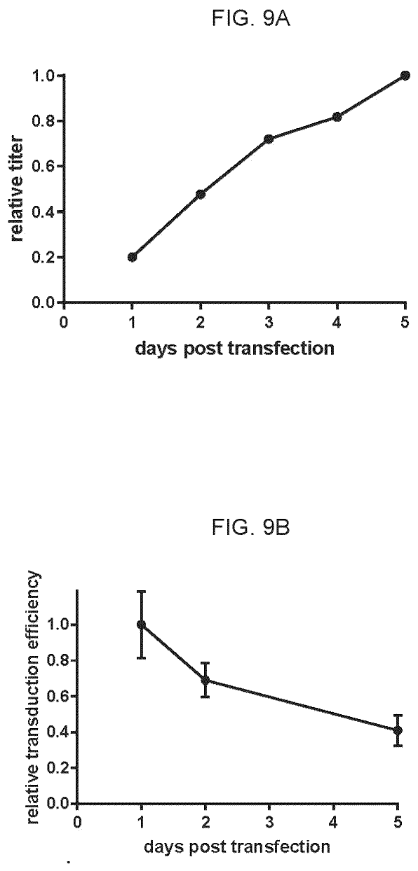

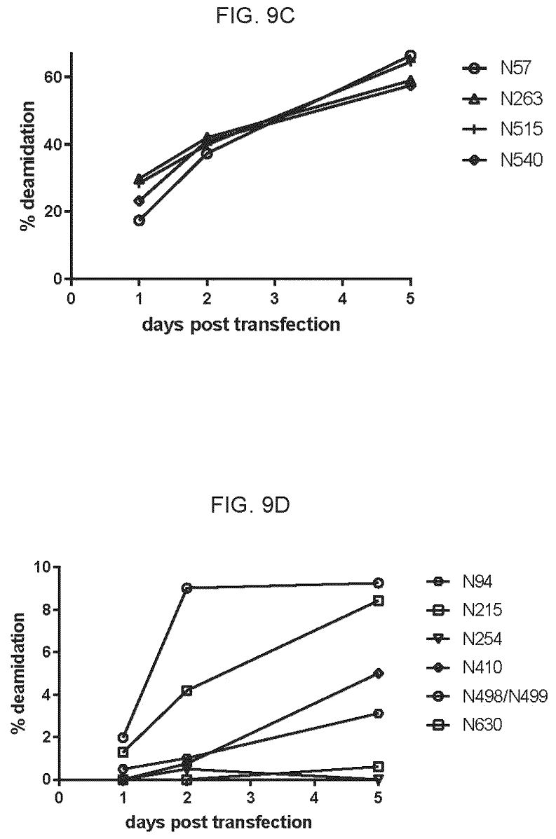

Given the apparently short half-life of NG deamidation, we reasoned that vector samples differing in age by as little as 1 day could show distinct deamidation profiles, thus providing an opportunity to correlate endogenous deamidation to function. Our large-scale vector preparation protocol calls for triple transfection of 293 cells followed by 5 days of incubation for vector production and 1-2 days for vector purification. To approximate this process, we prepared medium scale triple transfections (10×15 cm cell culture dishes each) of 293 cells with wt AAV8. We collected vector (2×15 cm cell culture dishes/day) at 1 day intervals for 5 days, preserving the timepoints until the end of the 5 day period by freezing vector at −80 C. Next, we assessed crude vector titer and in vitro transduction efficiency as described above. As expected, the number of assembled, DNAseI-resistant genome copies increased over time (FIG. 9A). We then quickly processed crude vector for early (day 1 and 2) and late (day 5) timepoints by affinity purification and measured in vitro transduction efficiency of huh7 cells. Relative transduction efficiency of the vector dropped progressively over time (FIG. 9B). In terms of transgene expression per GC added to target cells, day 5 vector was only 40% as efficient as day 1 material. This activity drop was observed for crude material as well, indicating a change in molecular composition before purification (FIG.). We observed a similar trend in activity loss for AAV9 over 5 days, with approximately 40% reduction in vector potency (FIG. 11I-FIG. 11K).

Next we measured deamidation of the time course samples by mass spectrometry. NG site deamidation progressed substantially over every interval, with an average of 25% deamidation at day 1, and >60% of sites converted by day 5 (FIG. 9C). Non-NG site deamidation generally progressed over 5 days, although at much lower levels and with less consistency between days 2 and 5 (FIG. 9D). The data correlates endogenous vector deamidation to an early timepoint decay in specific activity, and highlights a potential opportunity to capture more active vector by shortening the production cycle or finding capsid mutations that stabilize asparagines.

We note that the material used for mass spectrometry analysis in FIG. 2A-FIG. 2E was at least 7 days post-transfection, due to an additional 2 days for purification. The higher NG site deamidation in these samples (>80%) indicates that deamidation likely continues after the period of expression and during the recovery and purification processes at approximately the same rates until NG sites are completely deamidated or the vector sample is frozen. Thus deamidation is largely determined by the age of the vector and is not a process that is exclusive to or caused by the recovery and purification process. The much lower deamidation values in the day 1 material vs the day 5 material (both affinity purified) underscore this point.

### Stabilizing NG Asparagines can Improve Vector Performance

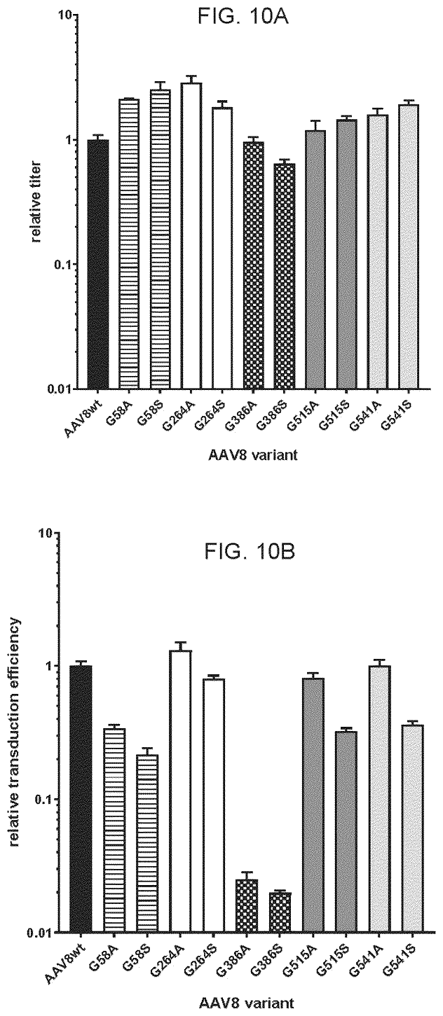

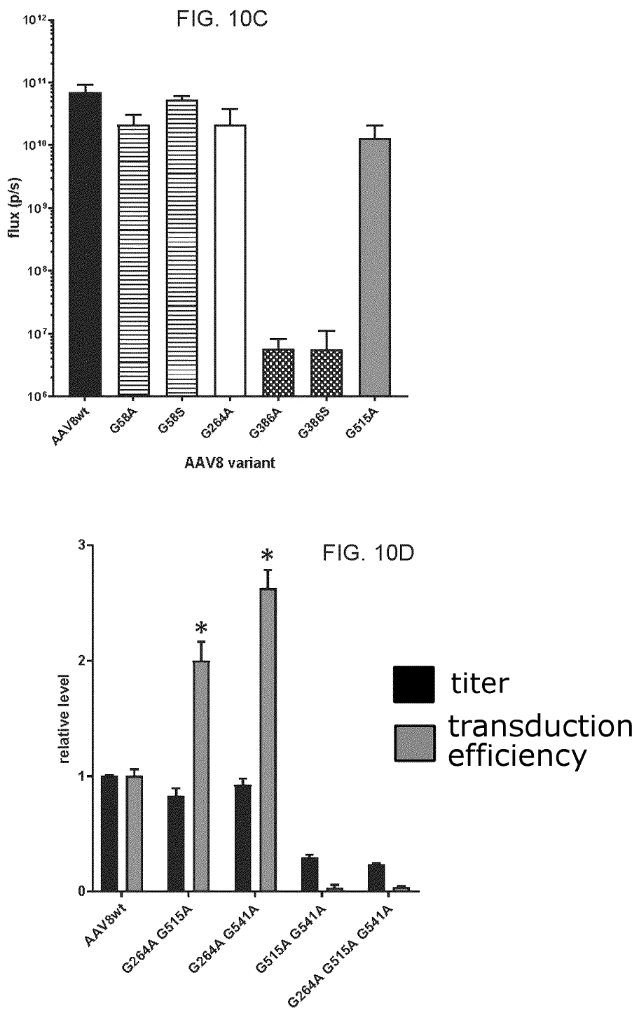

Given the correlation between vector NG deamidation and transduction efficiency loss, we reasoned that stabilizing NG amides by +1 site mutagenesis may improve vector function. We produced vector in small scale for AAV8 NG site mutants in which each +1 residue was individually converted to alanine or serine. Single +1 mutants were well tolerated in terms of vector assembly (FIG. 10A) and transduction efficiency (FIG. 10B). G386 substitutions, located near a previously defined “dead zone” on the capsid surface (Aydemir F, et al. J Virol July 2016; 90 (16): 7196-204), were defective for in vitro transduction. The loss of function for G386 mutants could indicate a preference for a deamidated asparagine at N385. Alternatively, the additional sidechain bulk at the +1 position may have a negative impact on function that is independent of amide-group stabilization. No single-site mutants significantly improved in vitro transduction, in spite of dramatic stabilization of their neighboring asparagines (Table 2). Because in vitro and in vivo transduction activities can be discordant, we tested a subset of the single-site +1 mutants for liver transduction in C57BL/6 mice. We performed intravenous tail vein injection (n=3 to 5) and examined luciferase expression by imaging weekly for 2 weeks (FIG. 10C). In vivo and in vitro transduction data were in agreement to within the associated errors of each assay (i.e., within the error range). G386 substitutions were defective for transduction, while +1 site mutations at other positions were largely tolerated, transducing liver at levels equivalent to but not exceeding wtAAV8.

Because stabilizing the amide at any one NG site may be necessary but not sufficient for functional restoration, we next evaluated vector variants with combinations of +1 site alanine substitutions. We recombined all 3 AAV8 NG sites for which the +1 alanine was highly functional (N263, N514, and N540). Some combinations, including the triple mutant G264A/G515A/G541A, assembled poorly and were dysfunctional for transduction. However, both pairwise combinations involving N263 (G246A/G515A and G264A/G541A) improved in vitro transduction efficiency (2.0- and 2.6-fold over wtAAV8, respectively) with no loss of titer (FIG. 10D). Because these mutations introduce at least two changes (N-amide stabilization and a +1 residue side chain substitution) these data do not conclusively link NG deamidation to functional loss. However, the data are consistent with the model established in the timecourse study in which NG site deamidation can impact in vitro transduction efficiency.

### Functional Asparagine Substitutions Improve Lot-to-Lot Reproducibility in Vector Manufacturing

Another potentially problematic aspect of the vector deamidation profiles we report is the high lot-to-lot variability in deamidation at some positions. For wtAAV8, this variability is most pronounced for N459 (observed deamidation ranging from 0% to 31%) and N499 (observed deamidation ranging from 0% to 53%). Variability in post-translational modifications is typically de facto avoided during biologics development, either by avoiding clones altogether that exhibit this variability, carefully monitoring and controlling production strains and conditions, or by protein engineering of the affected candidate.

As we were unable to determine the production or processing factors contributing to N459 and N499 deamidation variability (FIG. 2E), we sought functional amino acid substitutions at these positions. We first evaluated small scale vector preparations for conservative substitutions to glutamine at each position individually. Both N459Q and N499Q were assembled efficiently into vector, and were equivalent to the wtAAV8 reference for in vitro transduction efficiency (FIG. 7A). Next, we produced the mutants in large scale and performed mass spectrometry. Consistent with our observations of extremely rare glutamine deamidation, we observed selective and complete stabilization of the glutamine amides at positions 459 or 499 in these mutants (data not shown). We evaluated these mutant lots in vivo as above for liver transduction after tail vein injection in C57BL/6 mice (FIG. 7B and FIG. 7C). The wtAAV8 vector lot used as a control in this experiment was deamidated 16.8% at N499, but no deamidation was detected at N459 (data not shown). Liver transduction at day 14 for both mutants was equivalent to wtAAV8. This data demonstrates the potential for a protein engineering approach to address the molecular variability associated with deamidation in manufactured AAV vectors.

### C. Discussion

We identified and evaluated non-enzymatic deamidation of asparagine and glutamine residues on the AAV8 capsid independently by 2D gel electrophoresis, mass spectrometry, de novo protein modeling, and functional studies both in vitro and in vivo. Deamidation has been shown to occur in a wide variety of proteins and to significantly impact the activity of biologics, including antibody-based therapeutics (Nebija D et al. Int J Mol Sci 2014; 15 (4): 6399-411) and peptide-based vaccines (Verma A et al. Clin Vaccine Immunol. 2016; 23 (5): 396-402). Other viral proteins, such as the VP6 protein of rotavirus, have been shown by mass spectrometry to undergo deamidation events (Emslie K R et al. Funct Integr Genomics 2000; 1 (1): 12-24).

The context in which these deamidations occurred in AAV8 suggested that they are the result of spontaneous non-enzymatic events. Asparagine residues are known to be more extensively deamidated than glutamine residues; the amino acid downstream of the asparagine substantially influences the rate of deamidation with an N+1 of glycine (i.e., NG) being the most efficiently deamidated. We observed remarkable confirmation of the role of the N+1 amino acid in deamidation of AAV capsids in that every NG present in VP1 was deamidated at levels >75% while deamidation was never consistently >20% in any of the other asparagines or glutamines in the capsid. Virtually all NG motifs in the AAV8 and AAV9 capsids (i.e., 7/9) were also present on the surface of the capsid contained in HVR regions that are associated with high rates of conformational flexibility and thermal vibration. This is consistent with previous reports of NG motifs of other proteins that are located in regions where flexibility may be required for proper protein function and not in more ordered structures, such as alpha helices or beta sheets (Yan BX and Sun Y Q J Biol Chem 1997; 272 (6): 3190-4). The preference of NG motifs in surface exposed HVRs further enhances the rate of deamidation by providing solvent accessibility and conformational flexibility, thereby facilitating the formation of the succinimidyl intermediate. As predicted, less favorable environments lead to much lower rates of deamidation.

An important question regarding the biology of AAV and its use as a vector is the functional consequences of these deamidations. Mutagenesis of the capsid DNA to convert an asparagine to an aspartic acid allows for an evaluation of capsids in which all amino acids at a particular site are represented as aspartic acids. However, no easy strategy exists to use mutagenesis to prevent deamidations other than potentially mutating the N+1 residue, which is confounded by direct consequences of the second site mutation. We studied a limited number of variants in which the asparagine residue was converted to an aspartic acid by mutagenesis. Functional analysis included capsid assembly and in vitro and in vivo transduction. The most substantial effects of mutagenesis on vector function were those involving asparagines that were incompletely deamidated at baseline and were not surface exposed. What was surprising, however, was that mutagenesis of the highly deamidated asparagine at 514 to an aspartic acid did have some effect on function. This result suggests that the presence of residual amounts of the corresponding amide may influence function. This could be due in part to the presence of hydrogen bond interactions between N514 and D531 of another three-fold related VP3 monomer (identified in the wtAAV8 crystal structure) that are lost upon conversion of this residue to aspartic acid following deamidation.

A better understanding of the factors that influence the extent of deamidation in AAV vectors is important when assessing the impact of these deamidations on the development of novel therapeutics. Incubation of vectors under extreme conditions, known to markedly accelerate deamidation kinetics, had little effect. Coupled with our isotope incorporation studies, this result suggests that deamidation occurs during capsid assembly and is not an artifact of vector processing or mass spectrometry analysis. Deamidations at NG sites are unlikely to have substantive impact on vector performance, as the reaction was virtually complete in every sample that we evaluated. However, our initial functional studies suggest that residual amounts of non-deamidated asparagines can contribute to function. We are more concerned about sites where deamidation was less complete, which in most cases was also associated with sample-to-sample variation. An example is the asparagine at position 499 that showed deamidation ranging from 0% to 53% with a mean of 17%. It is possible that subtle differences in the conditions of vector production could contribute to this heterogeneity. The striking similarity in deamidation in AAV8 and AAV9 suggests this is a property of this entire family of viruses.

In summary, we discovered substantial heterogeneity in the primary amino acid structure of AAV8 and AAV9 capsid proteins. These studies potentially impact the development of AAV as vectors in several ways. First, the actual amino acid sequences of the VP proteins are not what are predicted by the corresponding DNA sequences. Second, aspects of the production method could lead to variations in deamidation and corresponding changes in vector function. Until we have a handle on the factors that influence deamidation rates at non-NG sites and a better understanding of their functional consequences it may be necessary to include deamidation in the characterization of clinical-grade AAV vectors. 2D gel electrophoresis can provide an overall assessment of net deamidation, although mass spectrometry will be necessary to assess deamidation at specific residues.

### Example 2: Deamidation AAV5.5.9

The novel sequences of AAV5.5.9 are provided in SEQ ID NO: 9 and 10, respectively. AAV5.5.9 vectors were assessed for deamidation as described in Example 1 for AAV9. Highly deamidated residues are seen at N57, N319, N442, N502.

| Modification | |
|---|---|
| SEQ ID NO: 10 | WL2019CS |
| Enzyme | Trypsin |
| % Coverage | 97.4 |
| N35 + Deamidation | 7.8 |
| ~N57 + Deamidation | 99.7 |
| N113 + Deamidation | 3.6 |
| ~N204 + Deamidation | 13.9 |
| N217 + Deamidation | 2.2 |
| ~N243 + Deamidation | 19.0 |
| Q249 + Deamidation | 11.4 |
| N293/294 + Deamidation | 37.3 |
| N304 + Deamidation | 6.2 |
| N309 + Deamidation | 0.7 |
| Q311 + Deamidation | 0.3 |
| ~N319 + Deamidation | 83.9 |
| N399/400 + Deamidation | 30.8 |
| ~N442 + Deamidation | 97.7 |
| N467 + Deamidation | 2.6 |
| N502 + Deamidation | 100.0 |
| N505 + Deamidation | 18.6 |
| ~Q589 + Deamidation | 21.1 |
| N618 + Deamidation | 6.6 |
| ~N641 + Deamidation | 8.1 |
| N653 + Deamidation | 8.3 |
| ~N658 + Deamidation | 21.7 |
| N694 + Deamidation | 0.6 |
| ~N699 + Deamidation | 8.6 |

### Example 3: Deamidation AAVrh79 (Clade E)

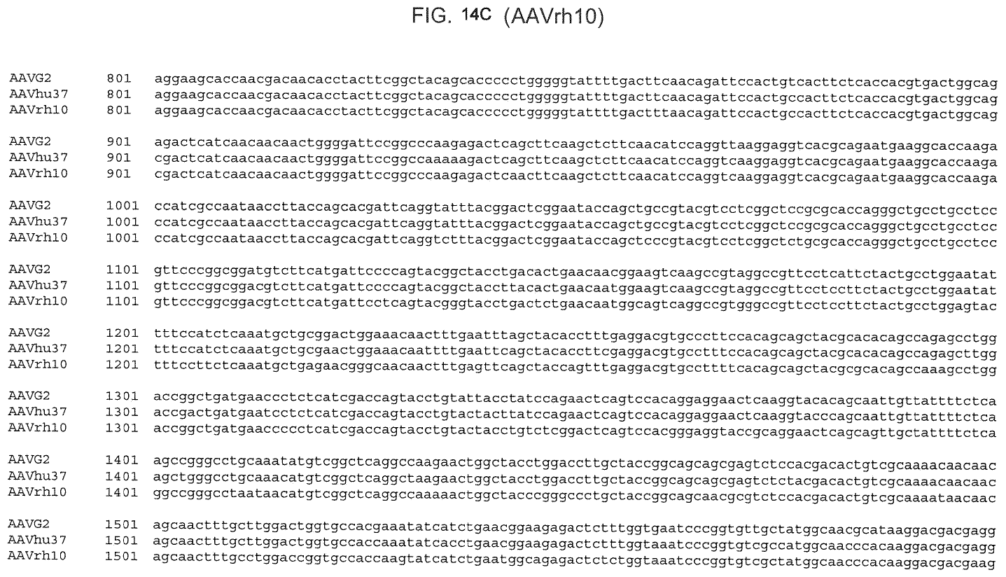

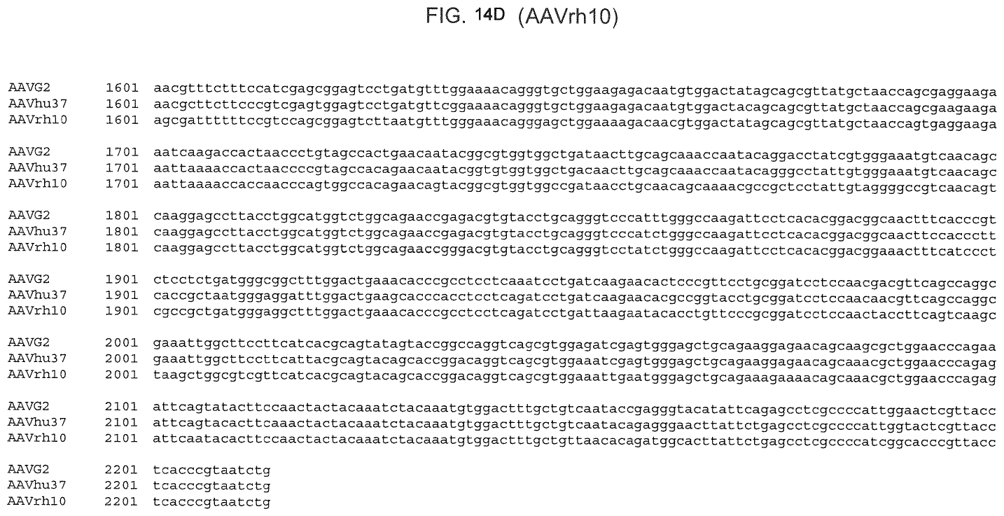

AAVrh79 was isolated from DNA extracted from small bowel tissue of rhesus macaque. It has been characterized phylogenetically as being within Clade E (FIG. 14A-14D). Its sequences are provided herein, with the nucleotide sequences being in SEQ ID NO: 1 and the amino acid sequence being in SEQ ID NO:2. An alignment of the amino acid sequences of AAVrh79, AAVrh. 10 and AAVhu.37 are provided in FIG. 14A. An alignment of the nucleic acid sequences of AAVrh79, AAVrh. 10 and AAVhu.37 are provided in FIG. 14B-14D.

AAVrh79 has three amino acid differences in its primary sequence. Whereas AAVhu37 has an Ala located at position 67 and a Lys at position 169 of its primary VP1 sequence, AAVrh79 has a glutamic acid (E) at position 67 and an Arg at position 169. Differences in the DNA sequences of VP1 among rh. 79, hu.37, and hu.40 are shown in FIG. 11B. Vectors expressing eGFP based on the various clade E variants were prepared and evaluated for their relative infectivity of Huh7 cells (FIG. 11C). C57BL/6 mice were injected with two dosage levels (3×10¹⁰ and 3×10¹¹ GC/mouse) of eGFP-expressing AAV8 or AAVrh.79 vectors and infectivity was assessed by fluorescence microscopy (Data not shown).

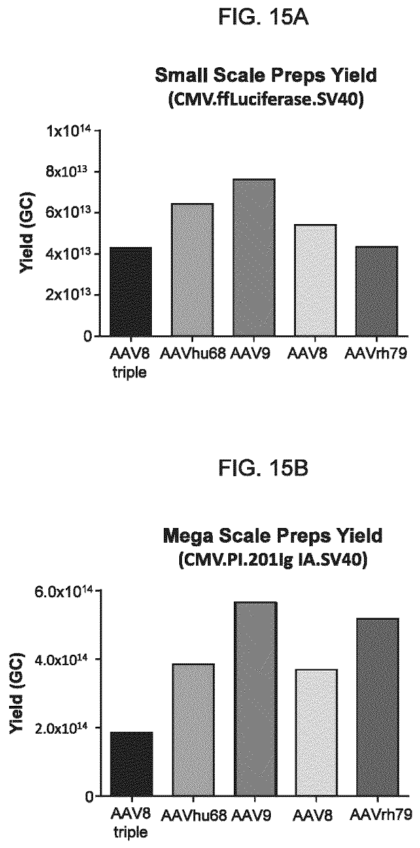

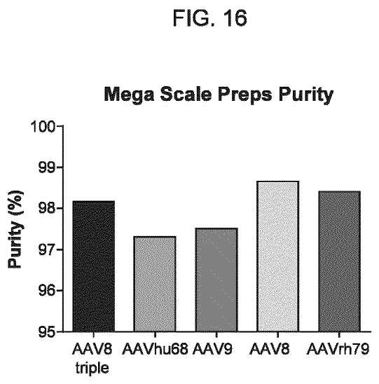

Vectors based on AAVrh79 were prepared using known production techniques using the AAVrh79 nucleotide sequence for production of the cap, such as previously described for AAV8 vectors. The results of the production yield and production purity assessments are provided in FIGS. 15A-15B and FIG. 16, respectively.

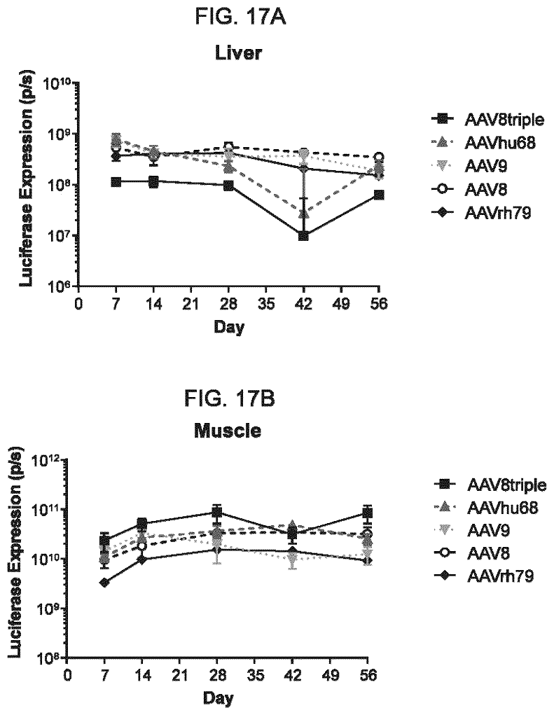

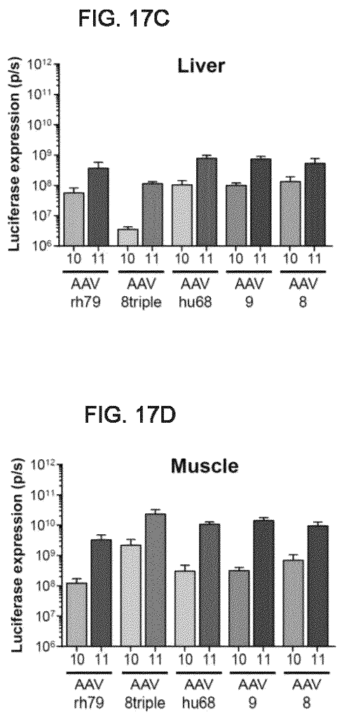

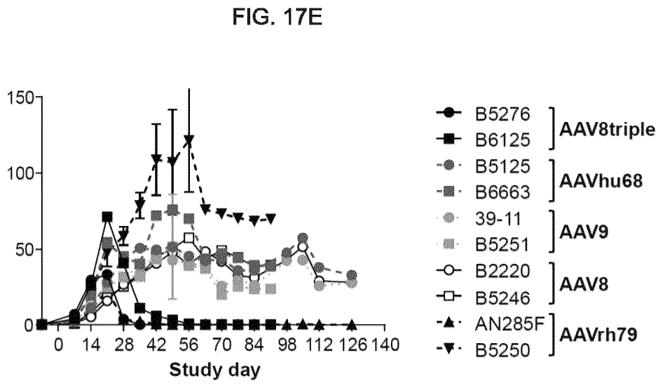

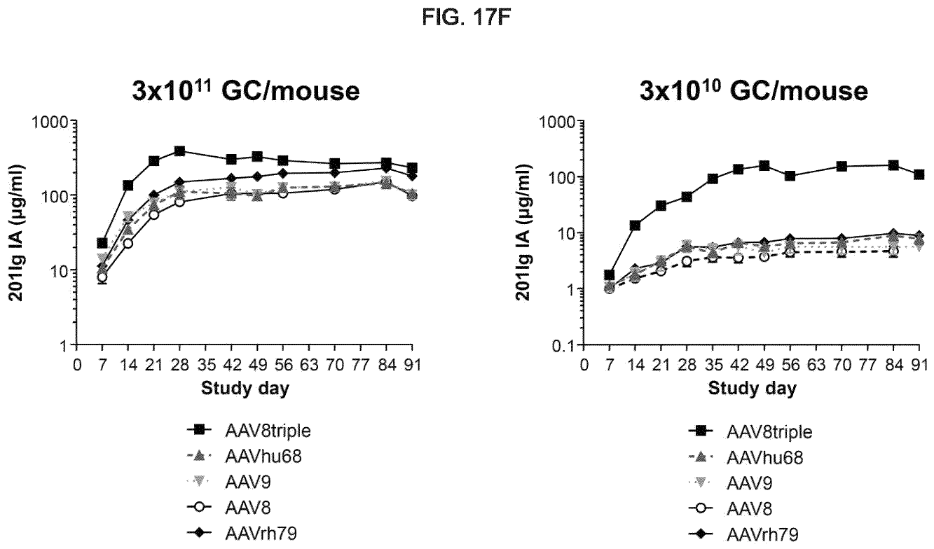

To assess expression levels using AAVrh79 containing a marker gene (firefly luciferase), male RAG KO mice at 6-8 weeks of age were injected intramuscularly with 3×10¹¹ GC/mouse of vector performed using a Hamilton syringe. ffLuc expression was visualized by whole-body bioluminescence imaging as previously described [Greig J A, Peng H, Ohlstein J, Medina-Jaszek C A, Ahonkhai O, Mentzinger A, et al. (2014) Intramuscular Injection of AAV8 in Mice and Macaques Is Associated with Substantial Hepatic Targeting and Transgene Expression. PLOS ONE 9 (11): e112268_doi_org/-10_1371/journal_pone_-0112268.] The results are provided in FIGS. 17A-17D.

Expression of AVV8triple, AAVhu68, AAV9, AAV8, and AAVrh79 vectors was compared following intramuscular administration of 10¹³ GC/kg AAVrh79 into male and female cynomolgus macaques (FIG. 17E).

Vectors expressing a secreted transgene (201Ig IA) were administrated intramuscularly into the gastrocnemius muscle of male RAG KO mice (n=5/group) (3×10¹⁰ or 3×10¹¹ GC/mouse). The results indicated that AAV8triple expresses better following 1M injection and at the lower dose tested the difference in expression from AAV8triple was substantial. At higher the higher dose, AVVrh79 expressed at levels at comparable to the other vectors tested (FIG. 17F).

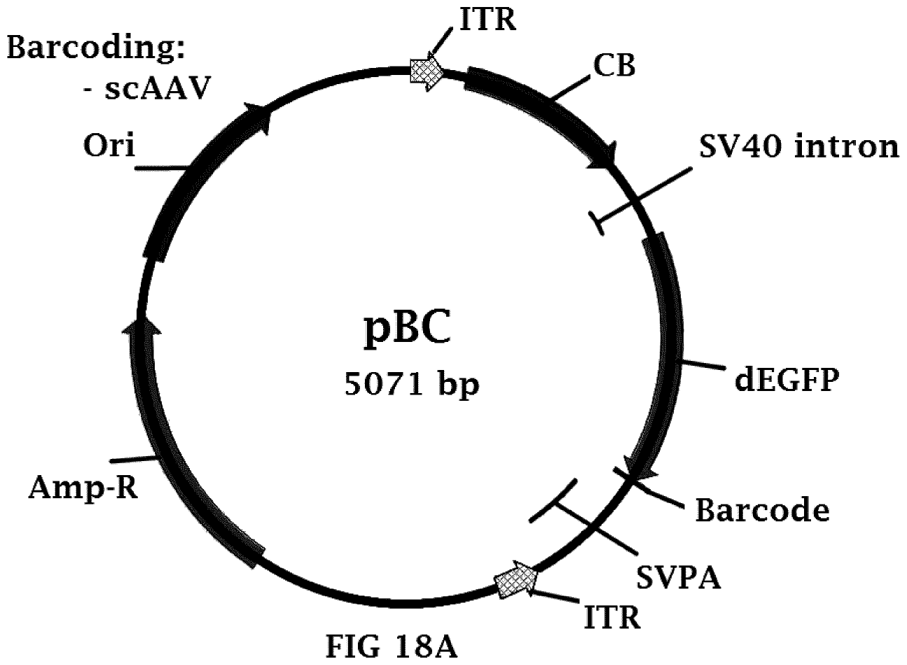

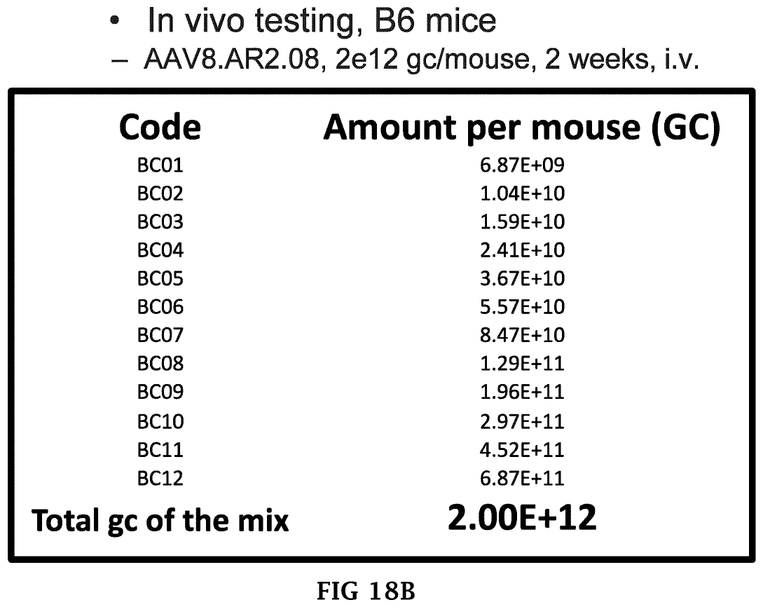

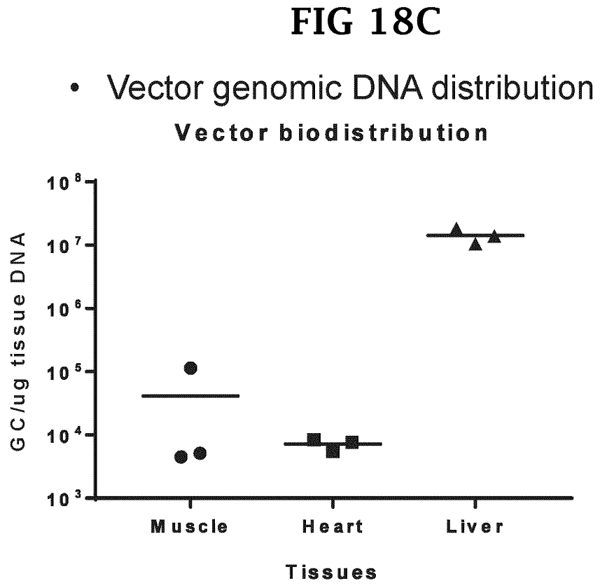

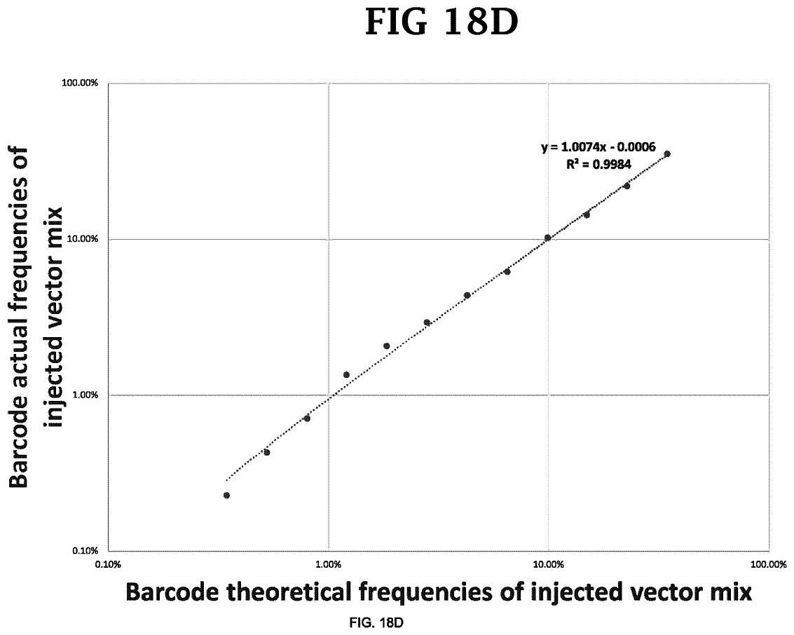

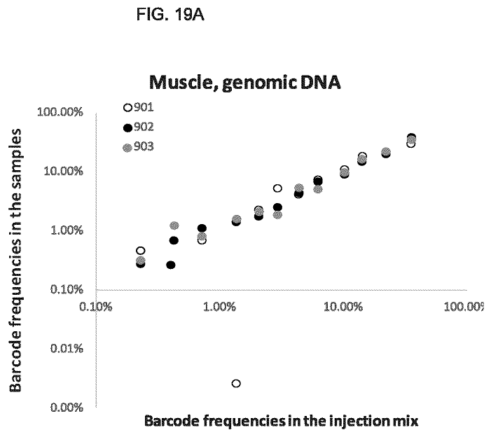

A female cynomolgus macaque (RA2362) was prescreened for NAbs (FIG. 18A) and injected with AAVG2.TBG.eGFP.WPRE.bGH (1×10¹³ ddGC/kg, intravenously). The animal was euthanatized 7 days following treatment, and a necropsy was performed to isolate the liver and other tissues for analysis. GFP expression in the liver and spleen was evaluated on day 7 (FIGS. 18B and 18C). Levels of vector detected in various tissues are shown in FIG. 18D. Levels of GFP expression were evaluated in the livers animals that received AAVG2 (RA2362) or AAV8 and AAVG3 vectors (FIG. 18E-FIG. 1J). FIG. 18K shows levels of vector detected in various tissues from these animals.

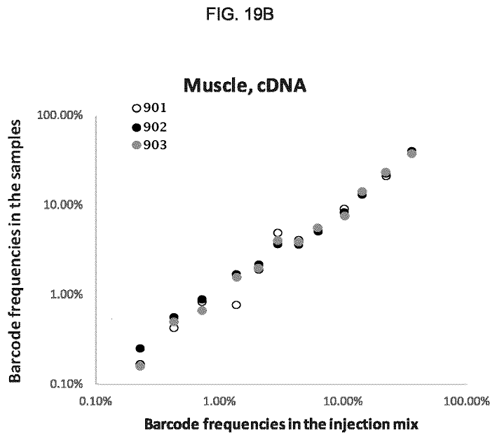

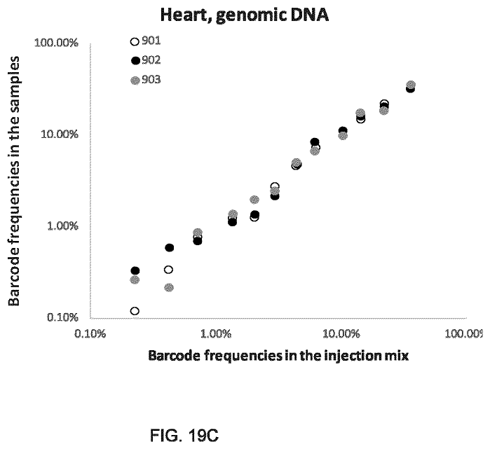

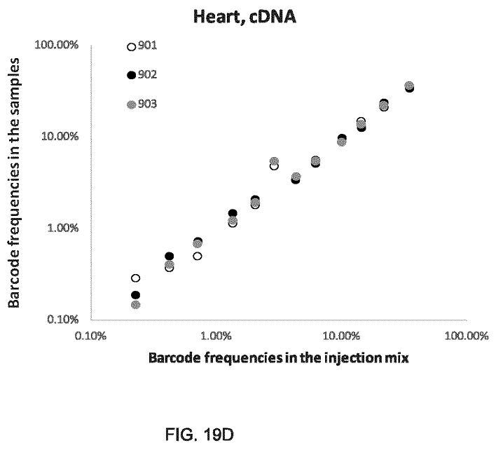

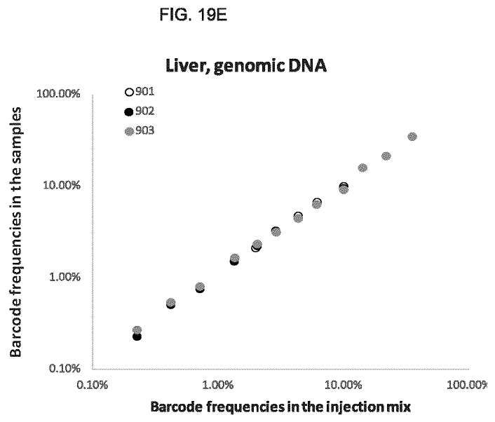

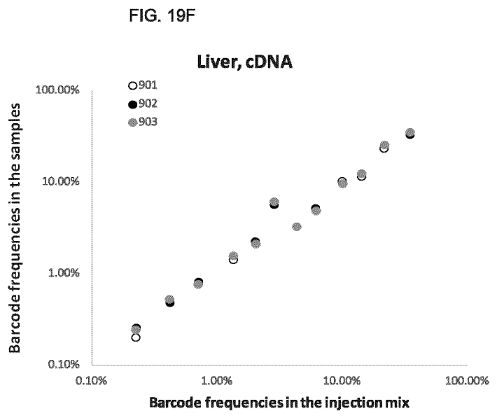

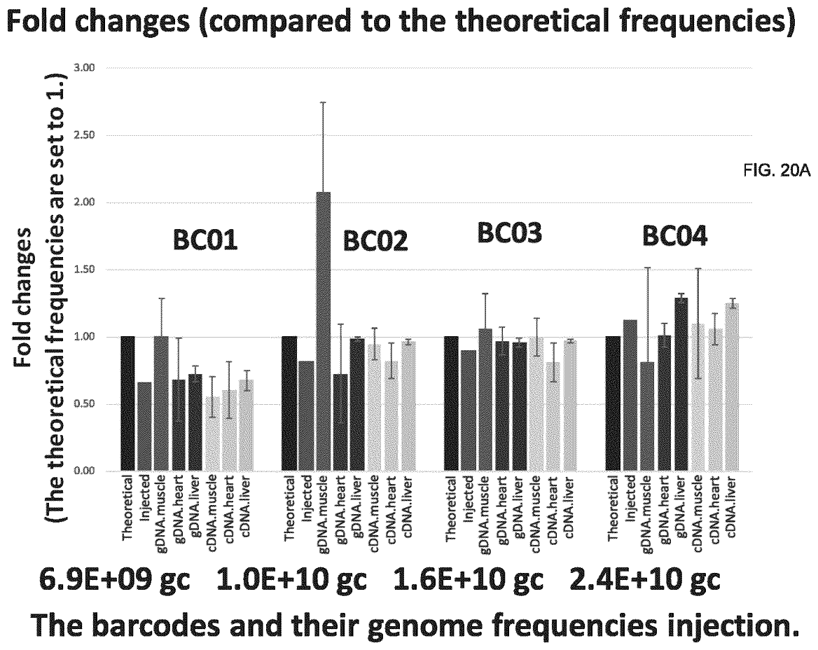

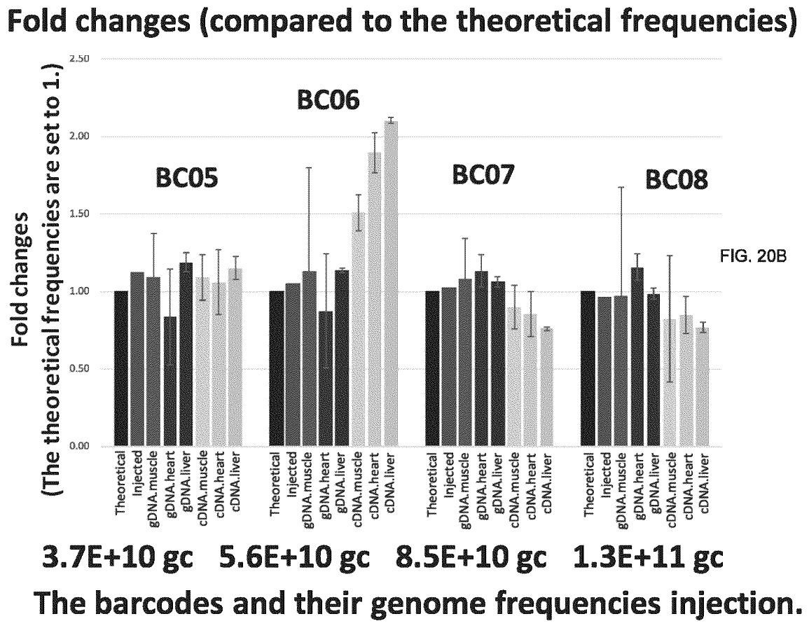

Various AAV8 and AAVrh79 vectors were generated and, in some instances, multiple lots were produced. The yields of these AAV9 and AAVrh79 vector lots were compared (FIG. 19).

AAVrh79 vectors were assessed for deamidation as described in Example 1 for AAV8 and AAV9. The results show that the vectors contain four amino acids which are highly deamidated (N57, N263, N385, N514), which correspond to asparagines in asparagine-glycine pairs, based on the numbering of AAVrh79 (SEQ ID NO: 1). Lower deamidation percentages are consistently observed in residues N94, N254, N410.

| Modification | | | | | | |
|---|---|---|---|---|---|---|
| AAVrh79 | | | | | | |
| SEQ ID NO: 2 | WL1781S | WL1784S | WL1785S | WL1781S | WL1784S | WL1785S |
| Enzyme | Trypsin | Trypsin | Trypsin | Chymotrypsin | Chymotrypsin | Chymotrypsin |
| % Coverage | 89.6 | 93.9 | 92.4 | 91.3 | 88.7 | 89.9 |
| N57 + | 99.3 | 80.9 | 82.9 | 99.6 | 80.1 | 86.4 |
| Deamidation | | | | | | |
| N94 + | 10.4 | 9.6 | 9.9 | 10.5 | 9.4 | 10.0 |
| Deamidation | | | | | | |
| ~N254 + | 16.0 | 15.8 | 16.4 | 15.3 | 16.3 | 16.7 |
| Deamidation | | | | | | |
| ~N263 + | 84.3 | 93.5 | 95.3 | 82.9 | 89.5 | 90.6 |
| Deamidation | | | | | | |
| ~N305 + | 3.2 | 2.5 | 2.4 | 3.2 | 2.6 | 2.3 |
| Deamidation | | | | | | |
| ~N385 + | 79.1 | 100.0 | 100.0 | 76.9 | 96.6 | 92.9 |
| Deamidation | | | | | | |
| ~N410 + | 2.0 | 17.8 | 23.9 | 2.0 | 17.6 | 23.0 |
| Deamidation | | | | | | |
| N479 + | 2.0 | 2.0 | 1.9 | 2.0 | 2.0 | 2.0 |
| Deamidation | | | | | | |
| ~N514 + | 100.0 | 97.2 | 97.0 | 97.4 | 94.6 | 98.1 |
| Deamidation | | | | | | |
| ~Q601 + | 0.1 | | | | | |
| Deamidation | | | | | | |
| N653 + | 1.3 | 1.1 | 1.4 | 1.3 | 1.1 | 1.5 |
| Deamidation | | | | | | |
| ~R487 + | 0.1 | 0.2 | 0.1 | 0.1 | 0.2 | 0.1 |
| Methylation | | | | | | |
| D97 + | 1.3 | | | 1.2 | | |
| Isomerization | | | | | | |
| S149 + | 51.9 | 49.0 | 53.2 | 49.6 | 46.6 | 55.8 |
| Phosphorylation | | | | | | |
| ~S153 + | 59.7 | 54.3 | 51.0 | 59.8 | 51.7 | 48.5 |
| Phosphorylation | | | | | | |
| ~S474 + | | 7.3 | 4.5 | | 7.0 | 4.3 |
| Phosphorylation | | | | | | |
| ~T570 + | 46.3 | 36.4 | 21.6 | 45.9 | 35.9 | 21.4 |
| Phosphorylation | | | | | | |
| ~S665 + | 0.5 | 0.3 | 0.4 | 0.4 | 0.3 | 0.4 |
| Phosphorylation | | | | | | |
| W248 + | | | 0.9 | | | |
| Oxidation | | | | | | |
| W307 + | 1.7 | 1.2 | 1.5 | 1.8 | 1.3 | 1.5 |
| Oxidation | | | | | | |
| W307 + | 0.3 | | 0.4 | | | |
| Oxidation to | | | | | | |
| kynurenine | | | | | | |
| M405 + | 5.8 | | | 6.0 | | |
| Oxidation | | | | | | |
| M437 + | 15.0 | 5.2 | 95.4 | 15.1 | 5.3 | 12.0 |
| Oxidation | | | | | | |
| M473 + | 16.7 | 7.4 | 7.9 | 16.0 | 7.6 | 7.6 |
| Oxidation | | | | | | |
| W480 + | 4.6 | 0.4 | 4.6 | 4.7 | 0.4 | 4.6 |
| Oxidation | | | | | | |
| W480 + | | | 0.1 | | | |
| Oxidation to | | | | | | |
| kynurenine | | | | | | |
| W505 + | 2.4 | 2.2 | 1.3 | 2.5 | 2.1 | 1.3 |
| Oxidation | | | | | | |
| M526 + | 19.0 | | | 18.6 | | |
| Oxidation | | | | | | |
| M544 + | 30.9 | 20.7 | | 31.4 | 20.3 | |
| Oxidation | | | | | | |
| M561 + | 15.6 | 7.7 | | 16.0 | 7.5 | |
| Oxidation | | | | | | |
| W621 + | | | 0.0 | | | |
| Oxidation to | | | | | | |
| kynurenine | | | | | | |
| M637 + | 6.9 | 12.5 | 4.5 | 6.9 | 12.6 | 4.7 |
| Oxidation | | | | | | |
| W697 + | | 0.5 | 0.6 | | 0.5 | 0.6 |
| Oxidation | | | | | | |

### Example 4: Preparation of AAV8.2.08

As discussed in WO 2017/180854 (incorporated herein by reference), several AAV8 mutants were generated c41, c42, c46, g110, g113, g115 and g117 with mutations in the HVR. VIII region. As discussed in Gurda et al, the major ADK8 epitope lies in the HVR. VIII region (amino acids 586 to 591 using AAV8 vp1 numbering). Those mutants were tested in vitro for ADK8 resistance and some of them were tested in vivo for ADK8 resistance. See, e.g., Lochrie 2006 cited above.

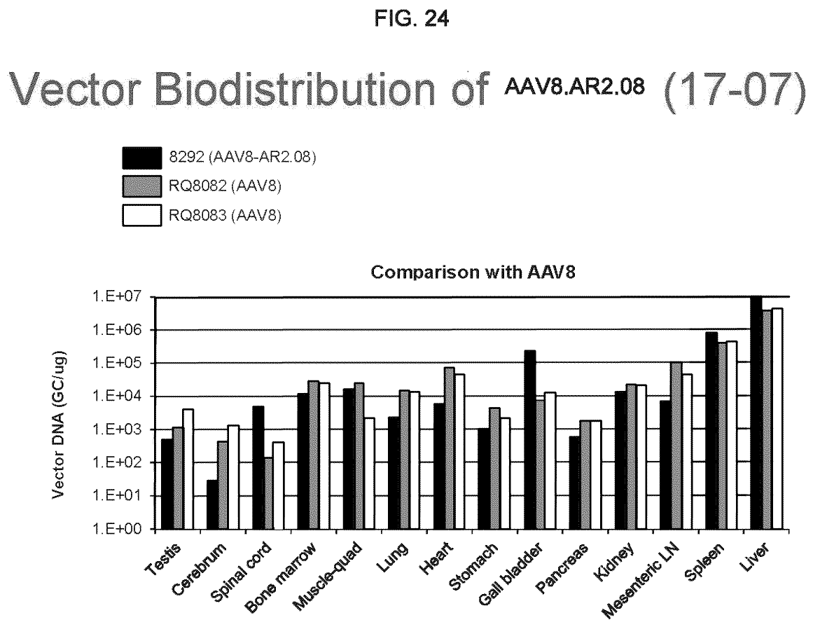

AR2.1-9 were randomly picked. AR2.25-61 were selected based on frequency. The randomly picked variants, as well as those higher-frequency ones, are alive, in terms of 6-well plate yield and Huh7 transduction. FIG. 24—shows expression in various tissues of AAV8.AR2.08 (left most set of bars).

### Example 5: Deamidation of AAV8.AR2.08

The novel sequences of AAV8.AR2.08 are provided in SEQ ID NO: 17 and 18, respectively, which were designed as in Example 4.

A. Modifications AAV8.AR2.08 vectors were produced and assessed for modifications as described in Example 1 for AAV8. The results show that the vectors contain five amino acids which are highly deamidated (N57, N263, N385, N514, and N540), which correspond to asparagines in asparagine-glycine pairs, based on the numbering of AAV8.AR2.08 (SEQ ID NO: 18). Lower deamidation percentages are consistently observed in residues N94, N254, N410. In contrast to AAV8, deamidation is not observed at position N459 (average 7% in AAV8) or N499 (average 17% in AAV8).

| AAV8.AR2.08 | | |
|---|---|---|
| Modification | | |
| SEQ ID NO: 18 | WL1846CS | WL1846CS |
| Enzyme | Trypsin | Chymotrypsin |
| % Coverage | 97.4 | 92.3 |
| N57 + Deamidation | 90.7 | 89.5 |
| N94 + Deamidation | 9.0 | 9.3 |
| ~N254 + Deamidation | 11.8 | 11.7 |
| ~N263 + Deamidation | 88.6 | 86.3 |
| ~N305 + Deamidation | 5.8 | 5.5 |
| ~N385 + Deamidation | 86.1 | 83.3 |
| ~N514 + Deamidation | 100.0 | 99.6 |
| ~N521 + Deamidation | 2.0 | 2.1 |
| ~N540 + Deamidation | 78.6 | 80.5 |
| N590 + Deamidation | 0.4 | 0.4 |
| Q601 + Deamidation | 0.5 | 0.6 |
| N653 + Deamidation | 0.8 | 0.8 |
| N665 + Deamidation | 1.2 | 1.2 |
| D442 + Isomerization | 12.0 | |
| D584 + Isomerization | 1.0 | |
| ~S149 + Phosphorylation | 95.9 | 15.4 |
| ~T417 + Phosphorylation | 0.0 | |
| ~T454 + Phosphorylation | 0.1 | |
| ~T493 + Phosphorylation | 0.1 | |
| S600 + Phosphorylation | 1.1 | |
| ~T663 + Phosphorylation | 0.0 | |
| ~W22 + Oxidation | 1.0 | 1.0 |
| ~M204 + Oxidation | 0.1 | 0.1 |
| ~M212 + Oxidation | 2.9 | 2.9 |
| W248 + Oxidation | 0.7 | 0.7 |
| W307 + Oxidation | 0.6 | 0.6 |
| M405 + Oxidation | 0.3 | 0.3 |
| M437 + Oxidation | 70.3 | 21.0 |
| M473 + Oxidation | 1.7 | 1.7 |
| W480 + Oxidation | 0.3 | 0.3 |
| W505 + Oxidation | 0.6 | 0.6 |
| M526 + Oxidation | 1.0 | 1.0 |
| M561 + Oxidation | 1.0 | 1.0 |
| M607 + Oxidation | 2.4 | 2.5 |
| ~W609 + Oxidation | 0.1 | 0.1 |
| W621 + Oxidation | 0.8 | 0.8 |
| M637 + Oxidation | 2.9 | 3.0 |
| W697 + Oxidation | 0.2 | 0.2 |

B. Single Cell RNA-Seq Reveals Tissue Localization and Transcriptional Signatures of Transduced Hepatocytes Isolated from Non-Human Primates Following Treatment with AAV8

Single cell RNA sequencing has proven to be a powerful technique to characterize the cellular transcriptome with unprecedented, single cell resolution. In our current work, we utilize single cell RNA-seq to study the transcriptional landscape of primary hepatocytes isolated from rhesus macaques following treatment with an AAV8 vector expressing GFP. Transcriptome analysis of FACS-sorted GFP+ and GFP− cells reveals tissue localization of transduced cells within the hepatic lobule as well as genes and regulatory pathways involved in hepatocyte transduction and the regulation of transgene expression.

For our study design, rhesus macaques were treated with either 1×10¹³ ddGC/kg AAV8.TBG.EGFP.WPRE (n=1) or 1×10¹³ ddGC/kg AAV8.2.08.TBG.EGFP.WPRE (AAV8 variant, n=1). Animals were euthanatized 7 days following treatment, and necropsies were performed to isolate the liver from both animals. Following treatment with collagenase and gradient centrifugation, isolated hepatocytes were FACS sorted by GFP transgene expression onto BD Precise™ 96 well plates. 192 single cells were isolated from each animal (96 GFP+ and 96 GFP−) and were subsequently used to prepare single cell RNA-seq libraries following the standard BD Precise™ protocol. Data were analyzed using the Seurat, Scran, and Scater packages in R in order to determine differentially expressed transcripts between GFP− and GFP+ sorted cells and to perform spatial reconstruction of isolated cells within the hepatic lobule using established transcriptional expression signatures.

AAV8.AR2.08 was found to have an increased liver tropism and exhibited a 1.5-fold increase in transduction efficiency as compared to AAV8. Single cell transcriptome analysis of sorted hepatocytes reveals transgene-expressing cells are evenly distributed across the hepatic lobule, showing a slight preference for the periportal region, which was also observed by histopathology. Interestingly, a subpopulation of sorted GFP− cells are found to express the transgene transcript at levels comparable to sorted GFP+ cells, suggesting that these cells are in fact transduced and express transgene mRNA, despite the absence of the detectable levels of translated protein. Comparing the transcriptional profiles of GFP− and GFP+ cells reveals differentially expressed transcripts involved in viral mRNA translation, elucidating possible pathways involved in the regulation of transgene protein expression in transduced cells.

C. Isolation of an Adeno-Associated Virus 8 Variant with Better Liver Transduction and Higher Liver Specificity in Nonhuman Primates with Directed Evolution Through a Human Liver Xenograft Model

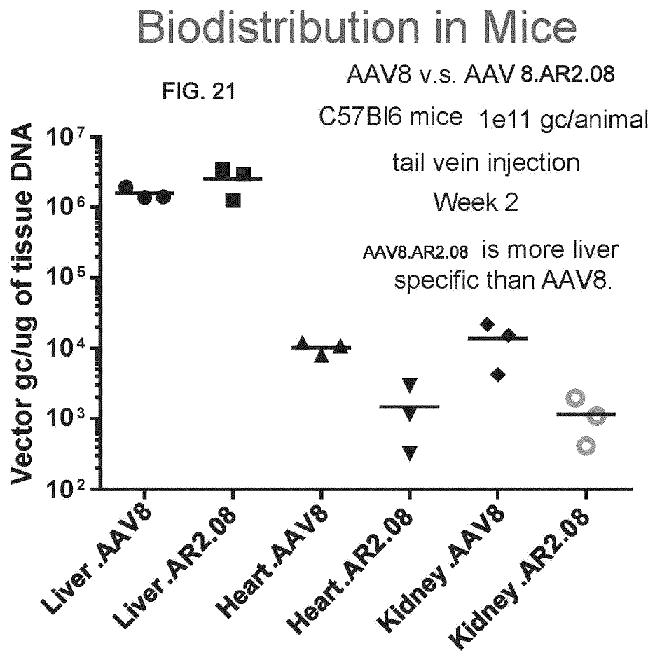

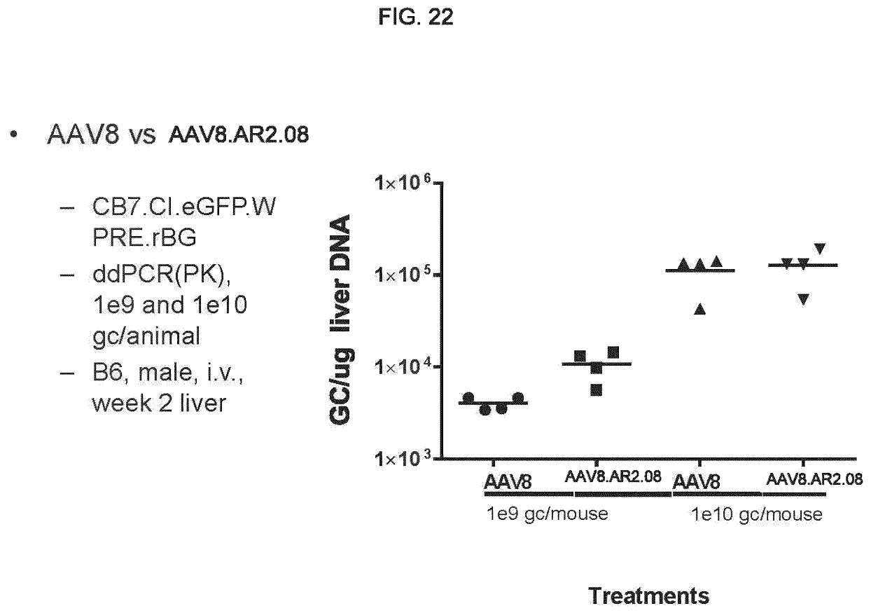

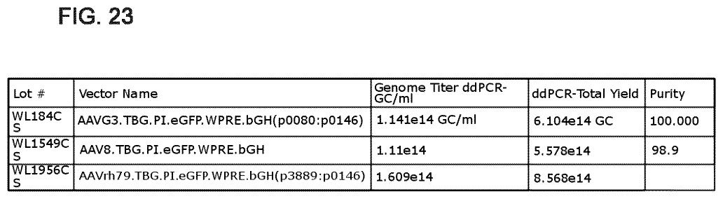

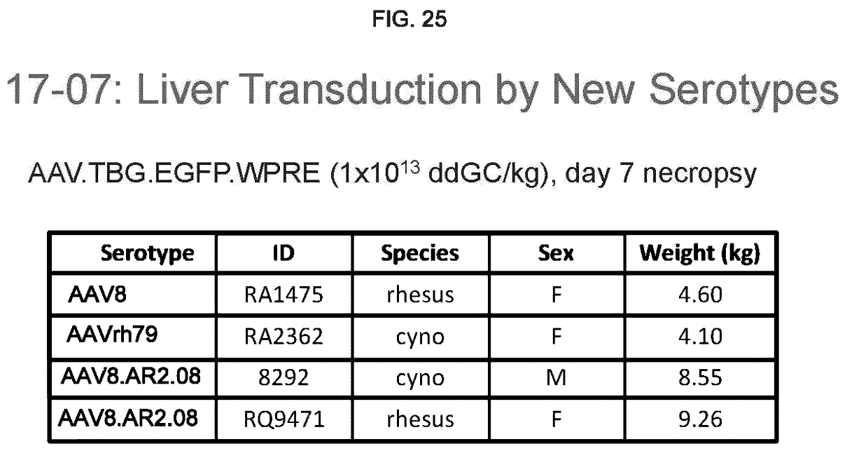

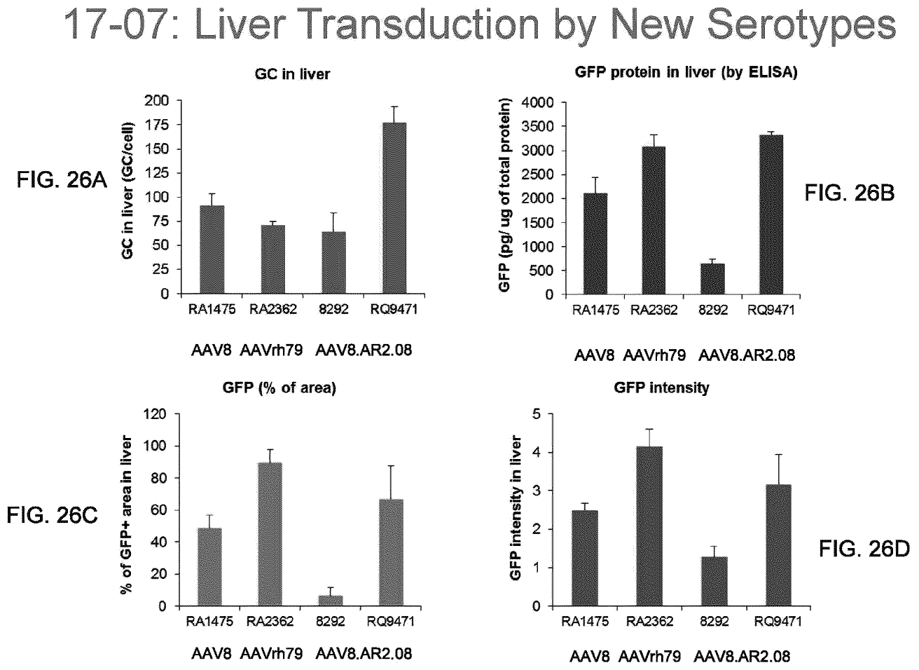

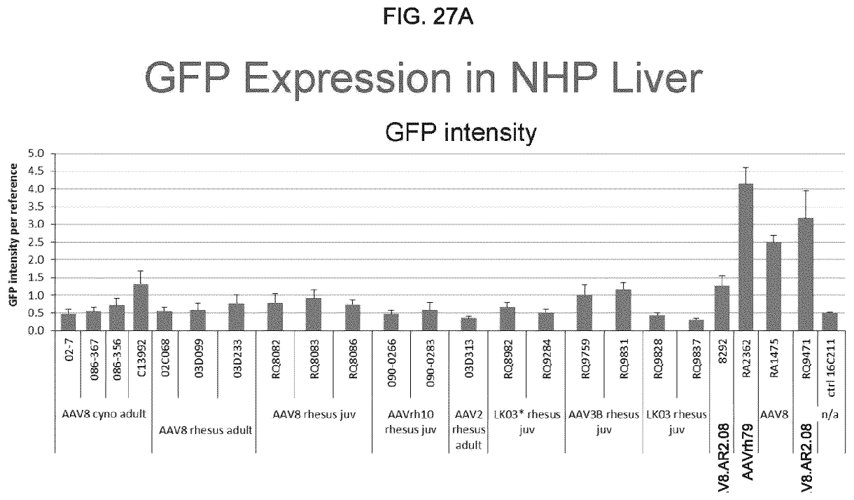

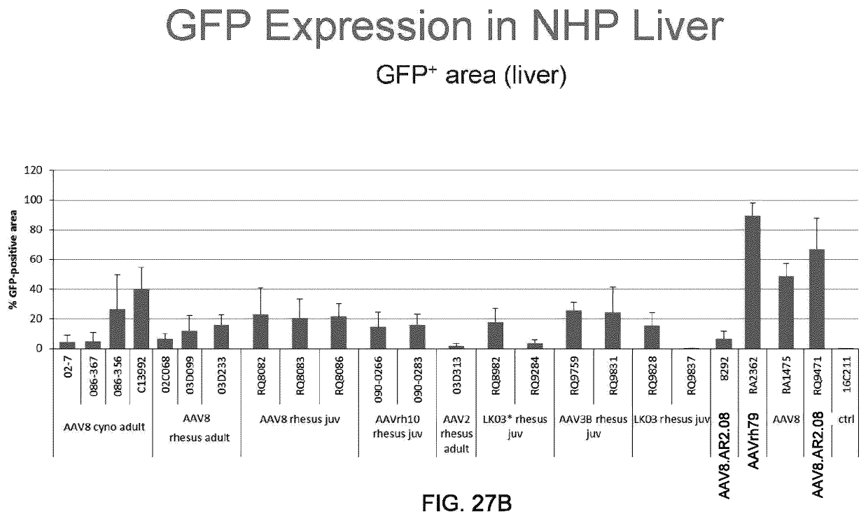

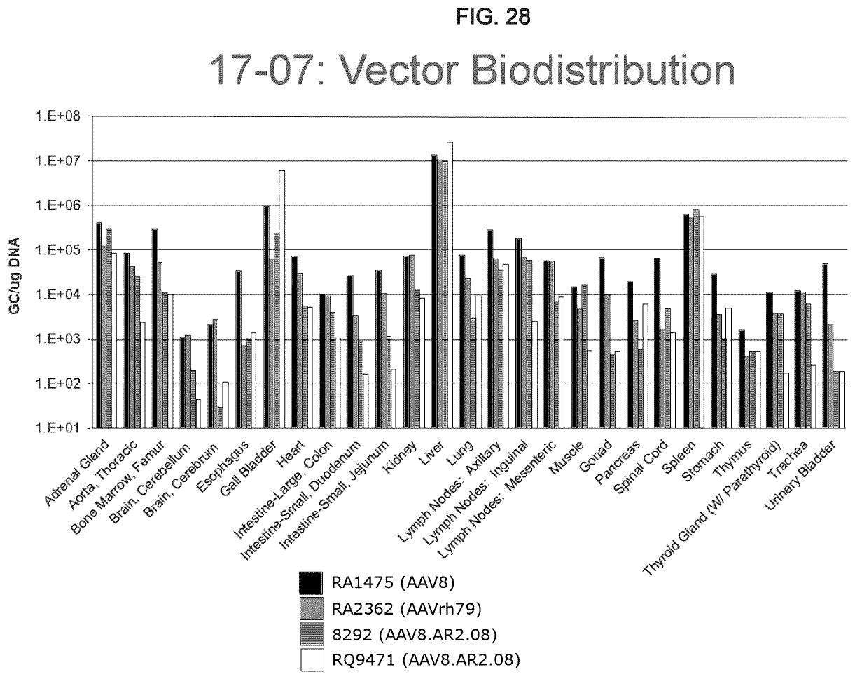

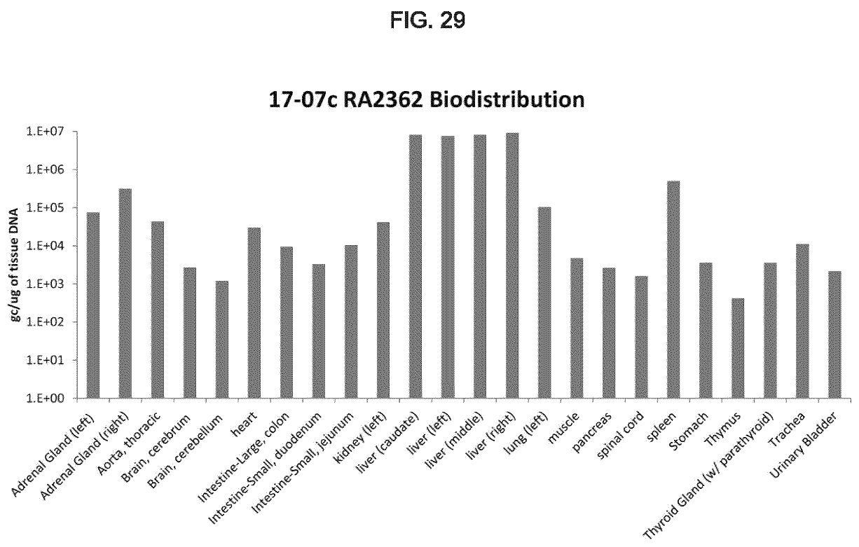

To derive AAV variants with better transduction and higher specificity, we performed AAV-directed evolution, using saturation mutagenesis targeting surface exposed sites on the capsid of AAV8, the benchmark for liver gene therapy, followed by two rounds of in vivo enrichment in human liver xenograft mouse model, and isolated an AAV8 variant called AAV8.2.08. After intravenous injection into nonhuman primates at a dose of 1e13 genome copies (GC)/kg body weight, vector genome copies delivered by AAV8.AR2.08 in various organs (including lung, heart, stomach, pancreas, kidney, and mesenteric lymph nodes) decreased while the delivery increased in liver, compared to AAV8, implying better liver transduction and higher tissue specificity. Next generation sequencing indicated significant enrichment of AAV8.AR2.08 during the in vivo selections, demonstrating the potential of the approach for isolating capsids with new and improved tropisms. Other comparisons are shown in FIGS. 21 to 27). FIG. 28 shows biodistribution of AAV8.AR2.08 and AAV8.

### D. Barcoding

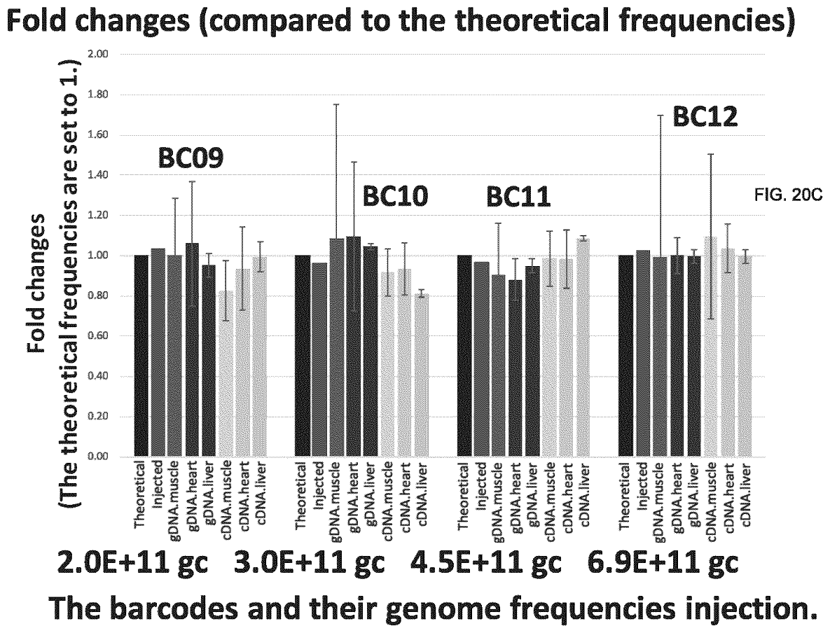

Black 6 mice were injected I.V. with 2×10¹² GC/mouse of a mixture of rAAVG3 derived from 12 preparations (FIG. 18B). Each preparation contains a separate barcode within the vector genome allowing identification of the specific preparation (FIG. 18A). After two weeks, animals were euthanized, and tissues harvested. As predicted, rAAVG3 expression was higher in liver than heart or muscle. FIG. 18C. Tissue distribution experiments show that actual frequencies match theoretical frequencies of barcodes in injected vector mix (FIG. 18D, total; FIG. 19A, 19B, muscle; FIG. 19C, 19D, heart; and FIG. 19E, 19F, liver), with slight anomalies in BC02 and BC06 (FIGS. 20A-20C).

All documents cited in this specification are incorporated herein by reference. U.S. Provisional Patent Application Nos. 62/722,388 and 62/722,382, both filed Aug. 24, 2018, U.S. Provisional Patent Application Nos. 62/703,670 and 62/703,673, both filed Jul. 26, 2018, U.S. Provisional Patent Application Nos. 62/677,471 and 62/677,474, both filed May 29, 2018, U.S. Provisional Patent Application No. 62/667,585, filed May 29, 2018, and U.S. Provisional Patent Application No. 62/635,964, filed Feb. 27, 2018 are incorporated herein by reference. U.S. Provisional Patent Application No. 62/667,881, filed May 7, 2018, U.S. Provisional Patent Application No. 62/667,888, filed May 7, 2018, U.S. Provisional Patent Application No. 62/667,587, filed May 6, 2018, US Provisional Patent Application No. 62/663,797, filed Apr. 27, 2018, U.S. Provisional Patent Application No. 62/663,788, filed Apr. 27, 2018, U.S. Provisional Patent application No. 62/635,968, filed Feb. 27, 2018 are incorporated by reference. The SEQ ID NO which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) which comprises:
(A) an AAVrh79 capsid comprising one or more of:
(1) AAVrh79 capsid proteins comprising:
a heterogeneous population of AAVrh79 vp1 proteins selected from: vp1 proteins produced by expression from a nucleic acid sequence which encodes the predicted amino acid sequence of 1 to 738 of SEQ ID NO:2, vp1 proteins produced from SEQ ID NO: 1, or vp1 proteins produced from a nucleic acid sequence at least 70% identical to SEQ ID NO: 1 which encodes the predicted amino acid sequence of 1 to 738 of SEQ ID NO:2,
a heterogeneous population of AAVrh79 vp2 proteins selected from: vp2 proteins produced by expression from a nucleic acid sequence which encodes the predicted amino acid sequence of at least about amino acids 138 to 738 of SEQ ID NO: 2, vp2 proteins produced from a sequence comprising at least nucleotides 412 to 2214 of SEQ ID NO:1, or vp2 proteins produced from a nucleic acid sequence at least 70% identical to at least nucleotides 412 to 2214 of SEQ ID NO:1 which encodes the predicted amino acid sequence of at least about amino acids 138 to 738 of SEQ ID NO:2,
a heterogeneous population of AAVrh79 vp3 proteins selected from: vp3 proteins produced by expression from a nucleic acid sequence which encodes the predicted amino acid sequence of at least about amino acids 204 to 738 of SEQ ID NO: 2, vp3 proteins produced from a sequence comprising at least nucleotides 607 to 2214 of SEQ ID NO:1, or vp3 proteins produced from a nucleic acid sequence at least 70% identical to at least nucleotides 607 to 2214 of SEQ ID NO: 1 which encodes the predicted amino acid sequence of at least about amino acids 204 to 738 of SEQ ID NO:2; and/or
(2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 738 of SEQ ID NO: 2, and a heterogeneous population of vp3 proteins which are the product of a nucleic acid sequence encoding at least amino acids 204 to 738 of SEQ ID NO:2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change; and
(B) a vector genome in the AAVrh79 capsid, the vector genome comprising a nucleic acid molecule comprising AAV inverted terminal repeat sequences and a non-AAV nucleic acid sequence encoding a product operably linked to sequences which direct expression of the product in a host cell.

2. The rAAV according to claim 1, wherein the deamidated asparagines are deamidated to aspartic acid, isoaspartic acid, an interconverting aspartic acid/isoaspartic acid pair, or combinations thereof.

3. The rAAV according to claim 1, wherein the capsid further comprises deamidated glutamine(s) which are deamidated to (α)-glutamic acid, γ-glutamic acid, an interconverting (α)-glutamic acid/γ-glutamic acid pair, or combinations thereof.

4. The rAAV according to claim 1, wherein the rAAVrh79 capsid further comprises at least subpopulation of vp1, vp2 and/or vp3 proteins having amino acid modifications from SEQ ID NO: 2 comprising at least about 50 to 100% deamidation at least four positions selected from one or more of N57, 263, 385, 514, or combinations thereof.

5. A recombinant adeno-associated virus (rAAV) which comprises:
(A) an AAV8.AR2.08 capsid comprising one or more of:
(1) AAV8.AR2.08 capsid proteins comprising:
a heterogeneous population of AAV8.AR2.08vp1 proteins selected from: vp1 proteins produced by expression from a nucleic acid sequence which encodes the predicted amino acid sequence of 1 to 738 of SEQ ID NO: 18, vp1 proteins produced from SEQ ID NO:17, or vp1 proteins produced from a nucleic acid sequence at least 70% identical to SEQ ID NO:17 which encodes the predicted amino acid sequence of 1 to 738 of SEQ ID NO: 18,
a heterogeneous population of AAV8.AR2.08vp2 proteins selected from: AAV8.AR2.08vp2 proteins produced by expression from a nucleic acid sequence which encodes the predicted amino acid sequence of at least about amino acids 138 to 738 of SEQ ID NO: 18, vp2 proteins produced from a sequence comprising at least nucleotides 411 to 2214 of SEQ ID NO: 17, or vp2 proteins produced from a nucleic acid sequence at least 70% identical to at least nucleotides 412 to 2214 of SEQ ID NO: 17 which encodes the predicted amino acid sequence of at least about amino acids 138 to 738 of SEQ ID NO: 18,
a heterogeneous population of AAV8.AR2.08vp3 proteins selected from: vp3 proteins produced by expression from a nucleic acid sequence which encodes the predicted amino acid sequence of at least about amino acids 204 to 738 of SEQ ID NO: 18, vp3 proteins produced from a sequence comprising at least nucleotides 607 to 2214 of SEQ ID NO:17, or vp3 proteins produced from a nucleic acid sequence at least 70% identical to at least nucleotides 607 to 2214 of SEQ ID NO:17 which encodes the predicted amino acid sequence of at least about amino acids 204 to 738 of SEQ ID NO:18; and/or
(2) a heterogeneous population of vp1 proteins which are the product of a nucleic acid sequence encoding the amino acid sequence of SEQ ID NO: 18, a heterogeneous population of vp2 proteins which are the product of a nucleic acid sequence encoding the amino acid sequence of at least about amino acids 138 to 738 of SEQ ID NO: 18, and a heterogeneous population of vp3 proteins which are the product of a nucleic acid sequence encoding at least amino acids 204 to 738 of SEQ ID NO:18 wherein: the vp1, vp2 and vp3 proteins contain subpopulations with amino acid modifications comprising at least two highly deamidated asparagines (N) in asparagine-glycine pairs in SEQ ID NO: 18 and optionally further comprising subpopulations comprising other deamidated amino acids, wherein the deamidation results in an amino acid change; and
(B) a vector genome in the AAV8.AR2.08capsid, the vector genome comprising a nucleic acid molecule comprising AAV inverted terminal repeat sequences and a non-AAV nucleic acid sequence encoding a product operably linked to sequences which direct expression of the product in a host cell.

6. The rAAV according to claim 5, wherein the deamidated asparagines are deamidated to aspartic acid, isoaspartic acid, an interconverting aspartic acid/isoaspartic acid pair, or combinations thereof.

7. The rAAV according to claim 5, wherein the capsid further comprises deamidated glutamine(s) which are deamidated to (α)-glutamic acid, γ-glutamic acid, an interconverting (α)-glutamic acid/γ-glutamic acid pair, or combinations thereof.

8. The rAAV according to claim 5, wherein the AAV8.AR2.08capsid comprises subpopulations having one or more of:
(a) at least 75% of asparagines (N) in asparagine-glycine pairs located at positions 57 of the vp1 proteins are deamidated, based on the numbering of SEQ ID NO: 18;
(b) at least 75% of N in asparagine-glycine pairs in position 263 of the vp1, v2 and vp3 proteins are deamidated, based on the residue numbering of the amino acid sequence of SEQ ID NO: 18,
(c) at least 70% of N in asparagine-glycine pairs in position 385 of the vp1, v2 and vp3 proteins are deamidated, based on the residue numbering of the amino acid sequence of SEQ ID NO: 18; and/or
(d) at least 85% of N in asparagine-glycine pairs in position 514 of the vp1, v2 and vp3 proteins are deamidated, based on the residue numbering of the amino acid sequence of SEQ ID NO: 18; and/or
(e) at least 85% of N in asparagine-glycine pairs in position 540 of the vp1, v2 and vp3 proteins are deamidated, based on the residue numbering of the amino acid sequence of SEQ ID NO: 18.

9. The rAAV according to claim 5, wherein the rAAV8.AR2.08capsid comprises a subpopulation of vp1 in which 75% to 100% of the N at position 57 of the vp1 proteins are deamidated, as determined using mass spectrometry.

10. The rAAV according to claim 5, wherein the rAAV8.AR2.08capsid comprises subpopulation of vp1 proteins, vp2 proteins, and/or vp3 proteins in which 75% to 100% of the N at position 263, based on the numbering of SEQ ID NO:2, are deamidated as determined using mass spectrometry.

11. The rAAV according to claim 5, wherein the rAAV8.AR2.08capsid comprises one or more of:
vp1 proteins, vp2 proteins, and/or vp3 proteins in which 75% to 100% of the N at position 385, based on the numbering of SEQ ID NO:2, are deamidated as determined using mass spectrometry; or
vp1 proteins, vp2 proteins, and/or vp3 proteins in which 75% to 100% of the N at position 514, based on the numbering of SEQ ID NO:2, are deamidated as determined using mass spectrometry; or
vp1 proteins, vp2 proteins, and/or vp3 proteins in which 75% to 100% of the N at position 540, based on the numbering of SEQ ID NO:2, are deamidated as determined using mass spectrometry.

12. The rAAV according to claim 5, wherein the nucleic acid sequence encoding the proteins is SEQ ID NO: 17, or a sequence at least 80% to at least 99% identical to SEQ ID NO: 17 which encodes the amino acid sequence of SEQ ID NO: 18.

13. The rAAV according to claim 12, wherein the nucleic acid sequence is at least 80% to 97% identical to SEQ ID NO: 17.

14. The rAAV according to claim 1, wherein the rAAV8.AR2.08capsid further comprises at least subpopulation of vp1, vp2 and/or vp3 proteins having amino acid modifications from SEQ ID NO: 18 comprising at least about 50 to 100% deamidation at least four positions selected from one or more of N57, 263, 385, 514, 540 or combinations thereof.

15. A recombinant adeno-associated virus (rAAV) which comprises:
(A) an AAV5.5.9 capsid comprising one or more of:
(1) AAV5.5.9 capsid proteins comprising:
a heterogeneous population of AAV5.5.9 vp1 proteins selected from: vp1 proteins produced by expression from a nucleic acid sequence which encodes the predicted amino acid sequence of 1 to 736 of SEQ ID NO: 10, vp1 proteins produced from SEQ ID NO:9, or vp1 proteins produced from a nucleic acid sequence at least 70% identical to SEQ ID NO:9 which encodes the predicted amino acid sequence of 1 to 726 of SEQ ID NO:1,
a heterogeneous population of AAV5.5.9 vp2 proteins selected from: AAV5.5.9 vp2 proteins produced by expression from a nucleic acid sequence which encodes the predicted amino acid sequence of at least about amino acids 137 to 726 of SEQ ID NO: 10, vp2 proteins produced from a sequence comprising at least nucleotides 409 to 2178 of SEQ ID NO:9, or vp2 proteins produced from a nucleic acid sequence at least 70% identical to at least nucleotides 409 to 2178 of SEQ ID NO:9 which encodes the predicted amino acid sequence of at least about amino acids 137 to 726 of SEQ ID NO:10,
a heterogeneous population of AAV5.5.9 vp3 proteins selected from: AAV5.5.9 vp3 proteins produced by expression from a nucleic acid sequence which encodes the predicted amino acid sequence of at least about amino acids 193 to 726 of SEQ ID NO: 10, vp3 proteins produced from a sequence comprising at least nucleotides 577 to 2178 of SEQ ID NO:9, or vp3 proteins produced from a nucleic acid sequence at least 70% identical to at least nucleotides 577 to 2178 of SEQ ID NO:9 which encodes the predicted amino acid sequence of at least about amino acids 193 to 726 of SEQ ID NO:10; and/or
(2) a heterogeneous population of vp1 proteins which are the product of a nucleic acid sequence encoding the amino acid sequence of SEQ ID NO: 10, a heterogeneous population of vp2 proteins which are the product of a nucleic acid sequence encoding the amino acid sequence of at least about amino acids 137 to 726 of SEQ ID NO: 10, and a heterogeneous population of vp3 proteins which are the product of a nucleic acid sequence encoding at least amino acids 193 to 726 of SEQ ID NO: 10 wherein: the vp1, vp2 and vp3 proteins contain subpopulations with amino acid modifications comprising at least two highly deamidated asparagines (N) in asparagine-glycine pairs in SEQ ID NO: 10 and optionally further comprising subpopulations comprising other deamidated amino acids, wherein the deamidation results in an amino acid change; and
(B) a vector genome in the AAV5.5.9 capsid, the vector genome comprising a nucleic acid molecule comprising AAV inverted terminal repeat sequences and a non-AAV nucleic acid sequence encoding a product operably linked to sequences which direct expression of the product in a host cell.

16. The rAAV according to claim 15, wherein the deamidated asparagines are deamidated to aspartic acid, isoaspartic acid, an interconverting aspartic acid/isoaspartic acid pair, or combinations thereof and/or the capsid further comprises deamidated glutamine(s) which are deamidated to (α)-glutamic acid, γ-glutamic acid, an interconverting (α)-glutamic acid/γ-glutamic acid pair, or combinations thereof.

17. The rAAV according to claim 15, wherein the AAV5.5.9 capsid comprises subpopulations having one or more of:
(a) at least 75% of asparagines (N) in asparagine-glycine pairs located at position 57 of the vp1 proteins are deamidated, based on the numbering of SEQ ID NO: 10;
(b) at least 70% of N in asparagine-glycine pairs in position 319 of the vp1, v2 and vp3 proteins are deamidated, based on the residue numbering of the amino acid sequence of SEQ ID NO: 10; and/or
(c) at least 85% of N in asparagine-glycine pairs in position 442 of the vp1, v2 and vp3 proteins are deamidated, based on the residue numbering of the amino acid sequence of SEQ ID NO: 10; and/or
(d) at least 85% of N in asparagine-glycine pairs in position 502 of the vp1, v2 and vp3 proteins are deamidated, based on the residue numbering of the amino acid sequence of SEQ ID NO: 10.

18. The rAAV according to claim 15, wherein the nucleic acid sequence encoding the proteins is SEQ ID NO: 9, or a sequence at least 80% to at least 99% identical to SEQ ID NO: 9 which encodes the amino acid sequence of SEQ ID NO: 10.

19. The rAAV according to claim 15, wherein the nucleic acid sequence is at least 80% to 97% identical to SEQ ID NO: 9.

20. The rAAV according to claim 1, wherein the AAV ITR sequences are a 5′ ITR and a 3′ ITR from an AAV source other than the AAV providing the capsid.

21. A composition comprising an AAV according to claim 1 and a pharmaceutical carrier, excipient and/or diluent.
